(19) United States
(12) Patent Application Publication
(10) Pub. No.: US 2022/0211871 A1
(43) Pub. Date: Jul. 7, 2022

(54) GENE THERAPIES FOR LYSOSOMAL DISORDERS

(71) Applicant: Prevail Therapeutics, Inc., New York, NY (US)

(72) Inventors: Asa Abeliovich, New York, NY (US); Laura Heckman, New York, NY (US); Herve Rhinn, New York, NY (US)

(73) Assignee: Prevail Therapeutics, Inc., New York, NY (US)

(21) Appl. No.: 17/601,984

(22) PCT Filed: Apr. 10, 2020
(86) PCT No.: PCT/US20/27658

§ 371 Date: Oct. 7, 2021

Related U.S. Application Data
(60) Provisional application No. 62/831,856, filed on Apr. 10, 2019.
(60) Provisional application No. 62/831,840, filed on Apr. 10, 2019.
(60) Provisional application No. 62/831,846, filed on Apr. 10, 2019.
(60) Provisional application No. 62/832,223, filed on Apr. 10, 2019.
(60) Provisional application No. 62/934,450, filed on Nov. 12, 2019.
(60) Provisional application No. 62/954,089, filed on Dec. 27, 2019.
(60) Provisional application No. 62/960,471, filed on Jan. 13, 2020.
(60) Provisional application No. 62/990,246, filed on Mar. 16, 2020.

(51) Int. Cl. A61K 48/00; C12N 9/24; C12N 15/86; C12N 15/113; C07K 14/705; C07K 14/475

(52) U.S. Cl. CPC A61K 48/005; C12N 9/2402; C12N 15/86; C12N 15/113; C07K 14/705; A61K 38/00; C12Y 302/01045; A01K 2207/20; A01K 2227/105; A01K 2267/0318; C07K 14/475

(57) ABSTRACT

The disclosure relates, in some aspects, to compositions and methods for treatment of central nervous system (CNS) diseases, for example Parkinson's disease (PD) and Gaucher disease. In some embodiments, the disclosure provides expression constructs comprising a transgene encoding one or more CNS disease-associated gene products and/or one or more an inhibitory nucleic acids targeting a CNS disease-associated gene or gene product. In some embodiments, the disclosure provides methods of treating CNS diseases by administering such expression constructs to a subject in need thereof.

### RELATED APPLICATIONS

This application is a national stage filing under 35 U.S.C. § 371 of international PCT application PCT/US2020/027658, filed Apr. 10, 2020, which claims priority under 35 U.S.C. § 119(e) to 62/832,223, filed Apr. 10, 2019, entitled “AAV VECTORS ENCODING TREM2 AND USES THEREOF”, 62/831,840, filed Apr. 10, 2019, entitled “GENE THERAPIES FOR LYSOSOMAL DISORDERS”, 62/831,846, filed Apr. 10, 2019, entitled “GENE THERAPIES FOR LYSOSOMAL DISORDERS”, 62/831,856, filed Apr. 10, 2019, entitled “GENE THERAPIES FOR LYSOSOMAL DISORDERS”, 62/934,450, filed Nov. 12, 2019, entitled “GENE THERAPIES FOR LYSOSOMAL DISORDERS”, 62/954,089, filed Dec. 27, 2019, entitled “GENE THERAPIES FOR LYSOSOMAL DISORDERS”, 62/960,471, filed Jan. 13, 2020, entitled “GENE THERAPIES FOR LYSOSOMAL DISORDERS”, 62/988,665, filed Mar. 12, 2020, entitled “GENE THERAPIES FOR LYSOSOMAL DISORDERS”, and 62/990,246, filed Mar. 16, 2020, entitled “GENE THERAPIES FOR LYSOSOMAL DISORDERS”, the entire contents of each of which are incorporated herein by reference.

### BACKGROUND

Gaucher disease is a rare inborn error of glycosphingolipid metabolism due to deficiency of lysosomal acid β-glucocerebrosidase (Gcase, “GBA”). Patients suffer from non-CNS symptoms and findings including hepatosplenomegly, bone marrow insufficiency leading to pancytopenia, lung disorders and fibrosis, and bone defects. In addition, a significant number of patients suffer from neurological manifestations, including defective saccadic eye movements and gaze, seizures, cognitive deficits, developmental delay, and movement disorders including Parkinson's disease.

Several therapeutics exist that address the peripheral disease and the principal clinical manifestations in hematopoietic bone marrow and viscera, including enzyme replacement therapies as described below, chaperone-like small molecule drugs that bind to defective Gcase and improve stability, and substrate reduction therapy that block the production of substrate that accumulate in Gaucher disease leading to symptoms and findings. However, other aspects of Gaucher disease (particularly those affecting the skeleton and brain) appear refractory to treatment.

### SUMMARY

The present disclosure relates, in part, to compositions and methods for treating certain central nervous system (CNS) diseases, for example neurodegenerative diseases (e.g., neurodegenerative diseases listed in Table 2), synucleinopathies (e.g., synucleinopathies listed in Table 3), tauopathies (tauopathies listed in Table 4), or lysosomal storage diseases (e.g., lysosomal storage diseases listed in Table 5).

In addition to Gaucher disease patients (who possess mutations in both chromosomal alleles of GBA1 gene), patients with mutations in only one allele of GBA1 are at highly increased risk of Parkinson's disease (PD). The severity of PD symptoms—which include gait difficulty, a tremor at rest, rigidity, and often depression, sleep difficulties, and cognitive decline—correlate with the degree of enzyme activity reduction. Thus, Gaucher disease patients have the most severe course, whereas patients with a single mild mutation in GBA1 typically have a more benign course. Mutation carriers are also at high risk of other PD-related disorders, including Lewy Body Dementia, characterized by executive dysfunction, psychosis, and a PD-like movement disorder, and multi-system atrophy, with characteristic motor and cognitive impairments. No therapies exist that alter the inexorable course of these disorders.

Deficits in enzymes such as Gcase (e.g., the gene product of GBA1 gene), as well as common variants in many genes implicated in lysosome function or trafficking of macromolecules to the lysosome (e.g., Lysosomal Membrane Protein 1 (LIMP), also referred to as SCARB2), have been associated with increased PD risk and/or risk of Gaucher disease (e.g., neuronopathic Gaucher disease, such as Type 2 Gaucher disease or Type 3 Gaucher disease). The disclosure is based, in part, on expression constructs (e.g., vectors) encoding one or more genes, for example Gcase, GBA2, prosaposin, progranulin, LIMP2, GALC, CTSB, SMPD1, GCH1, RAB7, VPS35, IL-34, TREM2, TMEM106B, or a combination of any of the foregoing (or portions thereof), associated with central nervous system (CNS) diseases, for example Gaucher disease, PD, etc. In some embodiments, combinations of gene products described herein act together (e.g., synergistically) to reduce one or more signs and symptoms of a CNS disease when expressed in a subject.

Accordingly, in some aspects, the disclosure provides an isolated nucleic acid comprising an expression construct encoding a Gcase (e.g., the gene product of GBA1 gene). In some embodiments, the isolated nucleic acid comprises a Gcase-encoding sequence that has been codon optimized (e.g., codon optimized for expression in mammalian cells, for example human cells). In some embodiments, the nucleic acid sequence encoding the Gcase encodes a protein comprising an amino acid sequence as set forth in SEQ ID NO: 14 (e.g., as set forth in NCBI Reference Sequence NP_000148.2). In some embodiments, the isolated nucleic acid comprises the sequence set forth in SEQ ID NO: 15. In some embodiments the expression construct comprises adeno-associated virus (AAV) inverted terminal repeats (ITRs), for example AAV ITRs flanking the nucleic acid sequence encoding the Gcase protein.

In some aspects, the disclosure provides an isolated nucleic acid comprising an expression construct encoding Prosaposin (e.g., the gene product of PSAP gene). In some embodiments, the isolated nucleic acid comprises a prosaposin-encoding sequence that has been codon optimized (e.g., codon optimized for expression in mammalian cells, for example human cells). In some embodiments, the nucleic acid sequence encoding the prosaposin encodes a protein comprising an amino acid sequence as set forth in SEQ ID NO: 16 (e.g., as set forth in NCBI Reference Sequence NP_002769.1). In some embodiments, the isolated nucleic acid comprises the sequence set forth in SEQ ID NO: 17. In some embodiments the expression construct comprises adeno-associated virus (AAV) inverted terminal repeats (ITRs), for example AAV ITRs flanking the nucleic acid sequence encoding the prosaposin protein.

In some aspects, the disclosure provides an isolated nucleic acid comprising an expression construct encoding LIMP2/SCARB2 (e.g., the gene product of SCARB2 gene). In some embodiments, the isolated nucleic acid comprises a SCARB2-encoding sequence that has been codon optimized (e.g., codon optimized for expression in mammalian cells, for example human cells). In some embodiments, the nucleic acid sequence encoding the LIMP2/SCARB2 encodes a protein comprising an amino acid sequence as set forth in SEQ ID NO: 18 (e.g., as set forth in NCBI Reference Sequence NP_005497.1). In some embodiments, the isolated nucleic acid comprises the sequence set forth in SEQ ID NO: 19. In some embodiments the expression construct comprises adeno-associated virus (AAV) inverted terminal repeats (ITRs), for example AAV ITRs flanking the nucleic acid sequence encoding the SCARB2 protein.

In some aspects, the disclosure provides an isolated nucleic acid comprising an expression construct encoding GBA2 protein (e.g., the gene product of GBA2 gene). In some embodiments, the isolated nucleic acid comprises a GBA2-encoding sequence that has been codon optimized (e.g., codon optimized for expression in mammalian cells, for example human cells). In some embodiments, the nucleic acid sequence encoding the GBA2 encodes a protein comprising an amino acid sequence as set forth in SEQ ID NO: 30 (e.g., as set forth in NCBI Reference Sequence NP_065995.1). In some embodiments, the isolated nucleic acid comprises the sequence set forth in SEQ ID NO: 31. In some embodiments the expression construct comprises adeno-associated virus (AAV) inverted terminal repeats (ITRs), for example AAV ITRs flanking the nucleic acid sequence encoding the GBA2 protein.

In some aspects, the disclosure provides an isolated nucleic acid comprising an expression construct encoding GALC protein (e.g., the gene product of GALC gene). In some embodiments, the isolated nucleic acid comprises a GALC-encoding sequence that has been codon optimized (e.g., codon optimized for expression in mammalian cells, for example human cells). In some embodiments, the nucleic acid sequence encoding the GALC encodes a protein comprising an amino acid sequence as set forth in SEQ ID NO: 33 (e.g., as set forth in NCBI Reference Sequence NP_000144.2). In some embodiments, the isolated nucleic acid comprises the sequence set forth in SEQ ID NO: 34. In some embodiments the expression construct comprises adeno-associated virus (AAV) inverted terminal repeats (ITRs), for example AAV ITRs flanking the nucleic acid sequence encoding the GALC protein.

In some aspects, the disclosure provides an isolated nucleic acid comprising an expression construct encoding CTSB protein (e.g., the gene product of CTSB gene). In some embodiments, the isolated nucleic acid comprises a CTSB-encoding sequence that has been codon optimized (e.g., codon optimized for expression in mammalian cells, for example human cells). In some embodiments, the nucleic acid sequence encoding the CTSB encodes a protein comprising an amino acid sequence as set forth in SEQ ID NO: 35 (e.g., as set forth in NCBI Reference Sequence NP_001899.1). In some embodiments, the isolated nucleic acid comprises the sequence set forth in SEQ ID NO: 36. In some embodiments the expression construct comprises adeno-associated virus (AAV) inverted terminal repeats (ITRs), for example AAV ITRs flanking the nucleic acid sequence encoding the CTSB protein.

In some aspects, the disclosure provides an isolated nucleic acid comprising an expression construct encoding SMPD1 protein (e.g., the gene product of SMPD1 gene). In some embodiments, the isolated nucleic acid comprises a SMPD1-encoding sequence that has been codon optimized (e.g., codon optimized for expression in mammalian cells, for example human cells). In some embodiments, the nucleic acid sequence encoding the SMPD1 encodes a protein comprising an amino acid sequence as set forth in SEQ ID NO: 37 (e.g., as set forth in NCBI Reference Sequence NP_000534.3). In some embodiments, the isolated nucleic acid comprises the sequence set forth in SEQ ID NO: 38. In some embodiments the expression construct comprises adeno-associated virus (AAV) inverted terminal repeats (ITRs), for example AAV ITRs flanking the nucleic acid sequence encoding the SMPD1 protein.

In some aspects, the disclosure provides an isolated nucleic acid comprising an expression construct encoding GCH1 protein (e.g., the gene product of GCH1 gene). In some embodiments, the isolated nucleic acid comprises a GCH1-encoding sequence that has been codon optimized (e.g., codon optimized for expression in mammalian cells, for example human cells). In some embodiments, the nucleic acid sequence encoding the GCH1 encodes a protein comprising an amino acid sequence as set forth in SEQ ID NO: 45 (e.g., as set forth in NCBI Reference Sequence NP_000534.3). In some embodiments, the isolated nucleic acid comprises the sequence set forth in SEQ ID NO: 46. In some embodiments the expression construct comprises adeno-associated virus (AAV) inverted terminal repeats (ITRs), for example AAV ITRs flanking the nucleic acid sequence encoding the GCH1 protein.

In some aspects, the disclosure provides an isolated nucleic acid comprising an expression construct encoding RAB7L protein (e.g., the gene product of RAB7L gene). In some embodiments, the isolated nucleic acid comprises a RAB7L-encoding sequence that has been codon optimized (e.g., codon optimized for expression in mammalian cells, for example human cells). In some embodiments, the nucleic acid sequence encoding the RAB7L encodes a protein comprising an amino acid sequence as set forth in SEQ ID NO: 47 (e.g., as set forth in NCBI Reference Sequence NP_003920.1). In some embodiments, the isolated nucleic acid comprises the sequence set forth in SEQ ID NO: 48. In some embodiments the expression construct comprises adeno-associated virus (AAV) inverted terminal repeats (ITRs), for example AAV ITRs flanking the nucleic acid sequence encoding the RAB7L protein.

In some aspects, the disclosure provides an isolated nucleic acid comprising an expression construct encoding VPS35 protein (e.g., the gene product of VPS35 gene). In some embodiments, the isolated nucleic acid comprises a VPS35-encoding sequence that has been codon optimized (e.g., codon optimized for expression in mammalian cells, for example human cells). In some embodiments, the nucleic acid sequence encoding the VPS35 encodes a protein comprising an amino acid sequence as set forth in SEQ ID NO: 49 (e.g., as set forth in NCBI Reference Sequence NP_060676.2). In some embodiments, the isolated nucleic acid comprises the sequence set forth in SEQ ID NO: 50. In some embodiments the expression construct comprises adeno-associated virus (AAV) inverted terminal repeats (ITRs), for example AAV ITRs flanking the nucleic acid sequence encoding the VPS35 protein.

In some aspects, the disclosure provides an isolated nucleic acid comprising an expression construct encoding IL-34 protein (e.g., the gene product of IL34 gene). In some embodiments, the isolated nucleic acid comprises a IL-34-encoding sequence that has been codon optimized (e.g., codon optimized for expression in mammalian cells, for example human cells). In some embodiments, the nucleic acid sequence encoding the IL-34 encodes a protein comprising an amino acid sequence as set forth in SEQ ID NO: 55 (e.g., as set forth in NCBI Reference Sequence NP_689669.2). In some embodiments, the isolated nucleic acid comprises the sequence set forth in SEQ ID NO: 56. In some embodiments the expression construct comprises adeno-associated virus (AAV) inverted terminal repeats (ITRs), for example AAV ITRs flanking the nucleic acid sequence encoding the IL-34 protein.

In some aspects, the disclosure provides an isolated nucleic acid comprising an expression construct encoding TREM2 protein (e.g., the gene product of TREM gene). In some embodiments, the isolated nucleic acid comprises a TREM2-encoding sequence that has been codon optimized (e.g., codon optimized for expression in mammalian cells, for example human cells). In some embodiments, the nucleic acid sequence encoding the TREM2 encodes a protein comprising an amino acid sequence as set forth in SEQ ID NO: 57 (e.g., as set forth in NCBI Reference Sequence NP_061838.1). In some embodiments, the isolated nucleic acid comprises the sequence set forth in SEQ ID NO: 58. In some embodiments the expression construct comprises adeno-associated virus (AAV) inverted terminal repeats (ITRs), for example AAV ITRs flanking the nucleic acid sequence encoding the TREM2 protein.

In some aspects, the disclosure provides an isolated nucleic acid comprising an expression construct encoding TMEM106B protein (e.g., the gene product of TMEM106B gene). In some embodiments, the isolated nucleic acid comprises a TMEM106B-encoding sequence that has been codon optimized (e.g., codon optimized for expression in mammalian cells, for example human cells). In some embodiments, the nucleic acid sequence encoding the TMEM106B encodes a protein comprising an amino acid sequence as set forth in SEQ ID NO: 63 (e.g., as set forth in NCBI Reference Sequence NP_060844.2). In some embodiments, the isolated nucleic acid comprises the sequence set forth in SEQ ID NO: 64. In some embodiments the expression construct comprises adeno-associated virus (AAV) inverted terminal repeats (ITRs), for example AAV ITRs flanking the nucleic acid sequence encoding the TMEM106B protein.

In some aspects, the disclosure provides an isolated nucleic acid comprising an expression construct encoding progranulin (e.g., the gene product of PGRN gene, also referred to as GRN gene). In some embodiments, the isolated nucleic acid comprises a prosaposin-encoding sequence that has been codon optimized (e.g., codon optimized for expression in mammalian cells, for example human cells). In some embodiments, the nucleic acid sequence encoding the progranulin (PRGN also referred to as GRN) encodes a protein comprising an amino acid sequence as set forth in SEQ ID NO: 67 (e.g., as set forth in NCBI Reference Sequence NP_002078.1). In some embodiments, the isolated nucleic acid comprises the sequence set forth in SEQ ID NO: 68. In some embodiments the expression construct comprises adeno-associated virus (AAV) inverted terminal repeats (ITRs), for example AAV ITRs flanking the nucleic acid sequence encoding the prosaposin protein.

Aspects of the disclosure relate to isolated nucleic acids and expression constructs (e.g., rAAV vectors) encoding one or more inhibitory nucleic acids. In some embodiments, one or more inhibitory nucleic acids target a gene associated with certain central nervous system (CNS) diseases (e,g, SNCA, TMEM106B, RPS2 or MAPT). In some embodiments, the inhibitory nucleic acids are expressed alone, or in combination with one or more gene products described herein (e.g., GBA1, PSAP, PRGN, etc.). In some embodiments, an isolated nucleic acid encodes 1) an inhibitory nucleic acid targeting SNCA, and 2) GBA1 protein. In some embodiments, an isolated nucleic acid encodes 1) an inhibitory nucleic acid targeting SNCA, and 2) PSAP protein. In some embodiments, an isolated nucleic acid encodes 1) an inhibitory nucleic acid targeting SNCA, and 2) PGRN protein (e.g., GRN protein). In some embodiments, an isolated nucleic acid encodes 1) an inhibitory nucleic acid targeting MAPT, and 2) GBA1 protein. In some embodiments, an isolated nucleic acid encodes 1) an inhibitory nucleic acid targeting MAPT, and 2) PSAP protein. In some embodiments, an isolated nucleic acid encodes 1) an inhibitory nucleic acid targeting MAPT, and 2) PGRN protein (e.g., GRN protein). In some embodiments, an isolated nucleic acid encodes 1) an inhibitory nucleic acid targeting TMEM106B, and 2) GBA1 protein. In some embodiments, an isolated nucleic acid encodes 1) an inhibitory nucleic acid targeting TMEM106B, and 2) PSAP protein. In some embodiments, an isolated nucleic acid encodes 1) an inhibitory nucleic acid targeting TMEM106B, and 2) PGRN protein (e.g., GRN protein). In some embodiments, an isolated nucleic acid encodes 1) an inhibitory nucleic acid targeting RPS25, and 2) GBA1 protein. In some embodiments, an isolated nucleic acid encodes 1) an inhibitory nucleic acid targeting RPS25, and 2) PSAP protein. In some embodiments, an isolated nucleic acid encodes 1) an inhibitory nucleic acid targeting RPS25, and 2) PGRN protein (e.g., GRN protein).

In some aspects, the disclosure provides an isolated nucleic acid comprising an expression construct encoding an inhibitory nucleic acid that inhibits expression or activity of α-Syn flanked by AAV inverted terminal repeats (ITRs). In some embodiments, the inhibitory nucleic acid is complementary to at least six contiguous nucleotides of the sequence set forth in SEQ ID NO: 90. In some embodiments, the inhibitory nucleic acid is an inhibitory RNA comprising the nucleic acid sequence set forth in any one of SEQ ID NOs: 20-25. In some embodiments, the inhibitory nucleic acid comprises the sequence set forth in any one of SEQ ID NOs: 94-99.

In some aspects, the disclosure provides an isolated nucleic acid comprising an expression construct encoding an inhibitory nucleic acid that inhibits expression or activity of TMEM106B flanked by AAV inverted terminal repeats (ITRs). In some embodiments, the inhibitory nucleic acid is complementary to at least six contiguous nucleotides of the sequence set forth in SEQ ID NO: 91. In some embodiments, the inhibitory nucleic acid is an inhibitory RNA comprising the nucleic acid sequence set forth in SEQ ID NO: 92 or 93. In some embodiments, the inhibitory nucleic acid comprises the sequence set forth in SEQ ID NO: 65 or 66.

In some aspects, the disclosure provides an isolated nucleic acid comprising an expression construct encoding an inhibitory nucleic acid that inhibits expression or activity of MAPT flanked by AAV inverted terminal repeats (ITRs). In some embodiments, the inhibitory nucleic acid is complementary to at least six contiguous nucleotides of the sequence set forth in SEQ ID NO: 114. In some embodiments, the inhibitory nucleic acid is an inhibitory RNA comprising the nucleic acid sequence set forth in SEQ ID NO: 123, 124, 127, 128, 131, 132, 135 or 136). In some embodiments, the inhibitory nucleic acid comprises the sequence set forth in SEQ ID NO: 125, 126, 129, 130, 133, 134, 137 or 138.

In some aspects, the disclosure provides an isolated nucleic acid comprising an expression construct encoding a first gene product and a second gene product, wherein each gene product independently is selected from the gene products, or portions thereof, set forth in Table 1 or an inhibitory nucleic acid targeting a gene or gene product set forth in Table 1. In some embodiments, the first gene product is a protein, and the second gene product is a protein. In some embodiments, the first gene product is an inhibitory nucleic acid and the second gene product is a protein. In some embodiments, the first gene product is an inhibitory nucleic acid and the second gene product is an inhibitory nucleic acid.

In some embodiments, the first gene product is a Gcase protein, or a portion thereof. In some embodiments, the second gene product is an inhibitory nucleic acid that targets SNCA. In some embodiments, the interfering nucleic acid is a siRNA, shRNA, miRNA, or dsRNA, optionally wherein the interfering nucleic acid inhibits expression of α-Syn protein. In some embodiments, the isolated nucleic acid further comprises one or more promoters, optionally wherein each of the one or more promoters is independently a chicken-beta actin (CBA) promoter, a CAG promoter, a CD68 promoter, or a JeT promoter. In some embodiments, the isolated nucleic acid further comprising an internal ribosomal entry site (IRES), optionally wherein the IRES is located between the first gene product and the second gene product. In some embodiments, the isolated nucleic acid further comprising a self-cleaving peptide coding sequence, optionally wherein the self-cleaving peptide is T2A. In some embodiments, the expression construct comprises two adeno-associated virus (AAV) inverted terminal repeat (ITR) sequences flanking the first gene product and the second gene product.

In some aspects, the disclosure provides an isolated nucleic acid comprising an expression construct encoding a first gene product and a second gene product, wherein each gene product independently is selected from the gene products, or portions thereof, set forth in Table 1 or an inhibitory nucleic acid targeting a gene or gene product set forth in Table 1.

In some embodiments, a first gene product or a second gene product is a Gcase protein, or a portion thereof. In some embodiments, a first gene product is a Gcase protein and a second gene product is selected from GBA2, prosaposin, progranulin, LIMP2, GALC, CTSB, SMPD1, GCH1, RAB7, VPS35, IL-34, TREM2, and TMEM106B.

In some embodiments, an expression construct encodes (e.g., alone or in addition to another gene product) an interfering nucleic acid (e.g., shRNA, miRNA, dsRNA, etc.). In some embodiments, an interfering nucleic acid inhibits expression of α-Synuclein (α-Synuclein). In some embodiments, an expression construct encodes an inhibitory nucleic acid targeting SNCA, and encodes one or more gene product selected from GBA1, GBA2, PSAP, PRGN, LIMP2, GALC, CTSB, SMPD1, GCH1, RAB7, VPS35, IL-34, TREM2, and TMEM106B. In some embodiments, an interfering nucleic acid that targets α-Synuclein comprises a sequence set forth in any one of SEQ ID NOs: 20-25. In some embodiments, an interfering nucleic acid that targets α-Synuclein binds to (e.g., hybridizes with) a sequence set forth in any one of SEQ ID NO: 20-25.

In some embodiments, an interfering nucleic acid inhibits expression of TMEM106B. In some embodiments, an expression construct encodes an inhibitory nucleic acid targeting TMEM106B, and encodes one or more gene product selected from GBA1, GBA2, PSAP, PRGN, LIMP2, GALC, CTSB, SMPD1, GCH1, RAB7, VPS35, IL-34, and TREM2. In some embodiments, an interfering nucleic acid that targets TMEM106B comprises a sequence set forth in SEQ ID NO: 65 or 66. In some embodiments, an interfering nucleic acid that targets TMEM106B binds to (e.g., hybridizes with) a sequence set forth in SEQ ID NO: 65 or 66.

In some embodiments, an interfering nucleic acid inhibits expression of MAPT. In some embodiments, an expression construct encodes an inhibitory nucleic acid targeting MAPT, and encodes one or more gene product selected from GBA1, GBA2, PSAP, PRGN, LIMP2, GALC, CTSB, SMPD1, GCH1, RAB7, VPS35, IL-34, TREM2, and TMEM106B. In some embodiments, an interfering nucleic acid that targets MAPT comprises a sequence set forth in any one of SEQ ID NOs: 123-138. In some embodiments, an interfering nucleic acid that targets MAPT binds to (e.g., hybridizes with) a sequence set forth in any one of SEQ ID NO: 123-138.

In some embodiments, an interfering nucleic acid inhibits expression of RPS25. In some embodiments, an expression construct encodes an inhibitory nucleic acid targeting RPS25, and encodes one or more gene product selected from GBA1, GBA2, PSAP, PRGN, LIMP2, GALC, CTSB, SMPD1, GCH1, RAB7, VPS35, IL-34, TREM2, and TMEM106B. In some embodiments, an interfering nucleic acid that targets RPS25 comprises a sequence set forth in any one of SEQ ID NOs: 115-122. In some embodiments, an interfering nucleic acid that targets RPS25 binds to (e.g., hybridizes with) a sequence set forth in any one of SEQ ID NO: 115-122. In some embodiments, an expression construct further comprises one or more promoters. In some embodiments, a promoter is a chicken-beta actin (CBA) promoter, a CAG promoter, a CD68 promoter, or a JeT promoter. In some embodiments, a promoter is a RNA pol II promoter (e.g., or an RNA pol III promoter (e.g., U6, etc.).

In some embodiments, an expression construct further comprises an internal ribosomal entry site (IRES). In some embodiments, an IRES is located between a first gene product and a second gene product.

In some embodiments, an expression construct further comprises a self-cleaving peptide coding sequence. In some embodiments, a self-cleaving peptide is a T2A peptide.

In some embodiments, an expression construct comprises two adeno-associated virus (AAV) inverted terminal repeat (ITR) sequences. In some embodiments, ITR sequences flank a first gene product and a second gene product (e.g., are arranged as follows from 5′-end to 3′-end: ITR-first gene product-second gene product-ITR). In some embodiments, one of the ITR sequences of an isolated nucleic acid lacks a functional terminal resolution site (trs). For example, in some embodiments, one of the ITRs is a ΔITR.

The disclosure relates, in some aspects, to rAAV vectors comprising an ITR having a modified “D” region (e.g., a D sequence that is modified relative to wild-type AAV2 ITR, SEQ ID NO: 29). In some embodiments, the ITR having the modified D region is the 5′ ITR of the rAAV vector. In some embodiments, a modified “D” region comprises an “S” sequence, for example as set forth in SEQ ID NO: 26. In some embodiments, the ITR having the modified “D” region is the 3′ ITR of the rAAV vector. In some embodiments, a modified “D” region comprises a 3′ITR in which the “D” region is positioned at the 3′ end of the ITR (e.g., on the outside or terminal end of the ITR relative to the transgene insert of the vector). In some embodiments, a modified “D” region comprises a sequence as set forth in SEQ ID NO: 26 or 27.

In some embodiments, an isolated nucleic acid (e.g., an rAAV vector) comprises a TRY region. In some embodiments, a TRY region comprises the sequence set forth in SEQ ID NO: 28.

In some embodiments, an isolated nucleic acid described by the disclosure comprises or consists of, or encodes a peptide having, the sequence set forth in any one of SEQ ID NOs: 1-149.

In some aspects, the disclosure provides a vector comprising an isolated nucleic acid as described by the disclosure. In some embodiments, a vector is a plasmid, or a viral vector. In some embodiments, a viral vector is a recombinant AAV (rAAV) vector or a Baculovirus vector. In some embodiments, an rAAV vector is single-stranded (e.g., single-stranded DNA).

In some aspects, the disclosure provides a host cell comprising an isolated nucleic acid as described by the disclosure or a vector as described by the disclosure.

In some aspects, the disclosure provides a recombinant adeno-associated virus (rAAV) comprising a capsid protein and an isolated nucleic acid or a vector as described by the disclosure.

In some embodiments, a capsid protein is capable of crossing the blood-brain barrier, for example an AAV9 capsid protein or an AAVrh.10 capsid protein. In some embodiments, an rAAV transduces neuronal cells and non-neuronal cells of the central nervous system (CNS).

In some aspects, the disclosure provides a method for treating a subject having or suspected of having or suspected of having a central nervous system (CNS) disease, the method comprising administering to the subject a composition (e.g., a composition comprising an isolated nucleic acid or a vector or a rAAV) as described by the disclosure. In some embodiments, the CNS disease is a neurodegenerative disease, such as a neurodegenerative disease listed in Table 2. In some embodiments, the CNS disease is a synucleinopathy, such as a synucleinopathy listed in Table 3. In some embodiments, the CNS disease is a tauopathy, such as a tauopathy listed in Table 4. In some embodiments, the CNS disease is a lysosomal storage disease, such as a lysosomal storage disease listed in Table 5. In some embodiments, the lysosomal storage disease is neuronopathic Gaucher disease, such as Type 2 Gaucher disease or Type 3 Gaucher disease.

In some embodiments, the disclosure relates to methods of treating a disease selected from Parkinson's Disease (e.g., Parkinson's Disease with GBA1 mutation (PD-GBA), sporadic Parkinson's Disease (sPD)), Gaucher Disease (e.g., neuronopathic Gaucher disease (nGD), Type I Gaucher Disease (T1GD), Type II Gaucher Disease (T2GD), and Type III Gaucher Disease (T3GD)), Dementia with Lewy Bodies (DLB), Amyotrophic lateral sclerosis (ALS), and Niemann-Pick Type C disease (NPC) by administering to a subject in need thereof an isolated nucleic acid (e.g., an rAAV vector or rAAV comprising an isolated nucleic acid) that encodes GBA1.

In some embodiments, the disclosure relates to methods of treating Frontotemporal Dementia (e.g., Frontotemporal Dementia with GRN mutation (FTD-GRN), Frontotemporal Dementia with MAPT mutation (FTD-tau), and Frontotemporal Dementia with C9ORF72 mutation (FTD-C9orf72)), Parkinson's Disease (PD), Alzheimer's Disease (AD), Neuronal Ceroid Lipofuscinosis (NCL), Corticobasal Degeneration (CBD), Motor Neuron Disease (MND), or Gaucher Disease (GD) by administering to a subject in need thereof an isolated nucleic acid (e.g., an rAAV vector or rAAV comprising an isolated nucleic acid) that encodes PGRN (e.g. GRN).

In some embodiments, the disclosure relates to methods of treating Synucleinopathies (e.g., multiple system atrophy (MSA), Parkinson's Disease (PD), Parkinson's disease with GBA1 mutation (PD-GBA), Dementia with Lewy Bodies (DLB), Dementia with Lewy Bodies with GBA1 mutation, and Lewy Body Disease) by administering to a subject in need thereof an isolated nucleic acid (e.g., an rAAV vector or rAAV comprising an isolated nucleic acid) that encodes GBA1 gene product, and an inhibitory nucleic acid targeting SNCA.

In some embodiments, the disclosure relates to methods of treating a disease selected from Parkinson's Disease (PD), Frontotemporal Dementia (e.g., Frontotemporal Dementia with GRN mutation (FTD-GRN)), Lysosomal Storage Diseases (LSDs), or Gaucher Disease (GD) by administering to a subject in need thereof an isolated nucleic acid (e.g., an rAAV vector or rAAV comprising an isolated nucleic acid) that encodes PSAP.

In some embodiments, the disclosure relates to methods of treating Alzheimer's Disease (AD), Nasu-Hakola Disease (NHD) or Parkinson's Disease (PD), by administering to a subject in need thereof an isolated nucleic acid (e.g., an rAAV vector or rAAV comprising an isolated nucleic acid) that encodes TREM2.

In some embodiments, the disclosure relates to methods of treating Alzheimer's disease (AD) or Frontotemporal Dementia (Frontotemporal Dementia with MAPT mutation (FTD-Tau), Progressive supranuclear palsy (PSP), neurodegenerative disease, Lewy Body Disease (LBD) or Parkinson's Disease by administering to a subject in need thereof an isolated nucleic acid (e.g., an rAAV vector or rAAV comprising an isolated nucleic acid) that encodes inhibitory nucleic acids targeting MAPT.

In some aspects, the disclosure provides a method for treating a subject having or suspected of having Parkinson's disease, the method comprising administering to the subject a composition (e.g., a composition comprising an isolated nucleic acid or a vector or a rAAV) as described by the disclosure.

In some embodiments, a composition comprises a nucleic acid (e.g., an rAAV genome, for example encapsidated by AAV capsid proteins) that encodes two or more gene products (e.g., CNS disease-associated gene products), for example 2, 3, 4, 5, or more gene products described in this application. In some embodiments, a composition comprises two or more (e.g., 2, 3, 4, 5, or more) different nucleic acids (e.g., two or more rAAV genomes, for example separately encapsidated by AAV capsid proteins), each encoding one or more different gene products. In some embodiments, two or more different compositions are administered to a subject, each composition comprising one or more nucleic acids encoding different gene products. In some embodiments, different gene products are operably linked to the same promoter type (e.g., the same promoter). In some embodiments, different gene products are operably linked to different promoters.

In some embodiments, administration comprises direct injection to the CNS of a subject. In some embodiments, direct injection is intracerebral injection, intraparenchymal injection, intrathecal injection, intra-cisterna manga injection, or any combination thereof. In some embodiments, direct injection to the CNS of a subject comprises convection enhanced delivery (CED).

In some embodiments, administration comprises peripheral injection. In some embodiments, peripheral injection is intravenous injection.

In some aspects, the present disclosure provides a method for treating a subject having or suspected of having a central nervous system (CNS) disease, the method comprising administering to the subject an isolated nucleic acid comprising: (i) an expression construct comprising a transgene encoding one or more gene products listed in Table 1 or an inhibitory nucleic acid targeting a gene or gene product set forth in Table 1; and (ii) two adeno-associated virus (AAV) inverted terminal repeats (ITRs) flanking the expression construct. In some aspects, the present disclosure provides a method for treating a subject having or suspected of having a central nervous system (CNS) disease, the method comprising administering to the subject two or more types of isolated nucleic acids encoding different gene products, where each type of isolated nucleic acid comprises: (i) an expression construct comprising a transgene encoding one or more gene products listed in Table 1 or an inhibitory nucleic acid targeting a gene or gene product set forth in Table 1; and (ii) two adeno-associated virus (AAV) inverted terminal repeats (ITRs) flanking the expression construct.

In some embodiments, the transgene encodes one or more proteins selected from: GBA1, GBA2, PGRN (e.g., GRN), TREM2, PSAP, SCARB2, GALC, SMPD1, CTSB, RAB7L, VPS35, GCH1, and IL34. In some embodiments, the transgene encoding one or more gene products comprises a codon-optimized protein coding sequence. In some embodiments, the transgene encodes one or more inhibitory nucleic acids targeting SNCA, MAPT, RPS25, and/or TMEM106B.

In some embodiments, the AAV ITRs are AAV2 ITRs.

In some embodiments, the isolated nucleic acid is packaged into a recombinant adeno-associated virus (rAAV). In some embodiments, the rAAV comprises an AAV9 capsid protein.

In some embodiments, the subject is a mammal. In some embodiments, the subject is a human. In some embodiments, the CNS disease is a neurodegenerative disease, synucleinopathy, tauopathy, and/or lysosomal storage disease (LSD). In some embodiments, the CNS disease is listed in Table 2, Table 3, Table 4, or Table 5.

In some embodiments, the administration comprises direct injection to the CNS of the subject, optionally wherein the direct injection is intracerebral injection, intraparenchymal injection, intrathecal injection, intra-cisterna magna injection or any combination thereof. In some embodiments, the intra-cisterna magna injection is suboccipital injection into the cisterna magna. In some embodiments, the direct injection to the CNS of the subject comprises convection enhanced delivery (CED). In some embodiments, the administration comprises peripheral injection, optionally wherein the peripheral injection is intravenous injection. In some embodiments, the subject is administered about 1×10¹⁰ vg to about 1×10¹⁶ vg of the rAAV.

### BRIEF DESCRIPTION OF THE DRAWINGS

FIG. 1 is a schematic depicting one embodiment of a vector comprising an expression construct encoding Gcase (e.g., GBA1 or a portion thereof).

FIG. 2 is a schematic depicting one embodiment of a vector comprising an expression construct encoding Gcase (e.g., GBA1 or a portion thereof) and LIMP2 (SCARB2) or a portion thereof. The coding sequences of Gcase and LIMP2 are separated by an internal ribosomal entry site (IRES).

FIG. 3 is a schematic depicting one embodiment of a vector comprising an expression construct encoding Gcase (e.g., GBA1 or a portion thereof) and LIMP2 (SCARB2) or a portion thereof. Expression of the coding sequences of Gcase and LIMP2 are each driven by a separate promoter.

FIG. 4 is a schematic depicting one embodiment of a vector comprising an expression construct encoding Gcase (e.g., GBA1 or a portion thereof), LIMP2 (SCARB2) or a portion thereof, and an interfering RNA for α-Syn.

FIG. 5 is a schematic depicting one embodiment of a vector comprising an expression construct encoding Gcase (e.g., GBA1 or a portion thereof), Prosaposin (e.g., PSAP or a portion thereof), and an interfering RNA for α-Syn.

FIG. 6 is a schematic depicting one embodiment of a vector comprising an expression construct encoding Gcase (e.g., GBA1 or a portion thereof) and Prosaposin (e.g., PSAP or a portion thereof). The coding sequences of Gcase and Prosaposin are separated by an internal ribosomal entry site (IRES).

FIG. 7 is a schematic depicting one embodiment of a vector comprising an expression construct encoding a Gcase (e.g., GBA1 or a portion thereof). In this embodiment, the vector comprises a CBA promoter element (CBA), consisting of four parts: the CMV enhancer (CMVe), CBA promoter (CBAp), Exon 1, and intron (int) to constitutively express the codon optimized coding sequence of human GBA1. The 3′ region also contains a WPRE regulatory element followed by a bGH polyA tail. Three transcriptional regulatory activation sites are included at the 5′ end of the promoter region: TATA, RBS, and YY1. The flanking ITRs allow for the correct packaging of the intervening sequences. Two variants of the 5′ ITR sequence (inset box) were evaluated; these have several nucleotide differences within the 20-nucleotide “D” region of wild-type AAV2 ITR. In some embodiments, an rAAV vector contains the “D” domain nucleotide sequence shown on the top line. In some embodiments, a rAAV vector comprises a mutant “D” domain (e.g., an “S” domain, with the nucleotide changes shown on the bottom line).

FIG. 8 is a schematic depicting one embodiment of the vector described in FIG. 6

FIG. 9 shows representative data for delivery of an rAAV comprising a transgene encoding a Gcase (e.g., GBA1 or a portion thereof) in a CBE mouse model of Parkinson's disease. Daily IP delivery of PBS vehicle, 25 mg/kg CBE, 37.5 mg/kg CBE, or 50 mg/kg CBE (left to right) initiated at P8. Survival (top left) was checked two times a day and weight (top right) was checked daily. All groups started with n=8. Behavior was assessed by total distance traveled in Open Field (bottom left) at P23 and latency to fall on Rotarod (bottom middle) at P24. Levels of the GCase substrates were analyzed in the cortex of mice in the PBS and 25 mg/kg CBE treatment groups both with (Day 3) and without (Day 1) CBE withdrawal. Aggregate GluSph and GalSph levels (bottom right) are shown as pmol per mg wet weight of the tissue. Means are presented. Error bars are SEM. *p<0.05; **p<0.01; ***p<0.001, nominal p-values for treatment groups by linear regression.

FIG. 10 is a schematic depicting one embodiment of a study design for maximal rAAV dose in a CBE mouse model. Briefly, rAAV was delivered by ICV injection at P3, and daily CBE treatment was initiated at P8. Behavior was assessed in the Open Field and Rotarod assays at P24-25 and substrate levels were measured at P36 and P38.

FIG. 11 shows representative data for in-life assessment of maximal rAAV dose in a CBE mouse model. At P3, mice were treated with either excipient or 8.8 e9 vg rAAV-GBA1 via ICV delivery. Daily IP delivery of either PBS or 25 mg/kg CBE was initiated at P8. At the end of the study, half the mice were sacrificed one day after their last CBE dose at P36 (Day 1) while the remaining half went through 3 days of CBE withdrawal before sacrifice at P38 (Day 3). All treatment groups (excipient+PBS n=8, rAAV-GBA1+PBS n=7, excipient+CBE n=8, and variant+CBE n=9) were weighed daily (top left), and the weight at P36 was analyzed (top right). Behavior was assessed by total distance traveled in Open Field at P23 (bottom left) and latency to fall on Rotarod at P24 (bottom right), evaluated for each animal as the median across 3 trials. Due to lethality, n=7 for the excipient+CBE group for the behavioral assays, while n=8 for all other groups. Means across animals are presented. Error bars are SEM. *p<0.05; ***p<0.001, nominal p-values for treatment groups by linear regression in the CBE-treated animals.

FIG. 12 shows representative data for biochemical assessment of maximal rAAV dose in a CBE mouse model. The cortex of all treatment groups (excipient+PBS n=8, variant+PBS n=7, excipient+CBE n=7, and variant+CBE n=9) was used to measure GCase activity (top left), GluSph levels (top right), GluCer levels (bottom left), and vector genomes (bottom right) in the groups before (Day 1) or after (Day 3) CBE withdrawal. Biodistribution is shown as vector genomes per 1 μg of genomic DNA. Means are presented. Error bars are SEM. (*)p<0.1; **p<0.01; ***p<0.001, nominal p-values for treatment groups by linear regression in the CBE-treated animals, with collection days and gender corrected for as covariates.

FIG. 13 shows representative data for behavioral and biochemical correlations in a CBE mouse model after administration of excipient+PBS, excipient+CBE, and variant+CBE treatment groups. Across treatment groups, performance on Rotarod was negatively correlated with GluCer accumulation (A, p=0.0012 by linear regression), and GluSph accumulation was negatively correlated with increased GCase activity (B, p=0.0086 by linear regression).

FIG. 14 shows representative data for biodistribution of variant in a CBE mouse model. Presence of vector genomes was assessed in the liver, spleen, kidney, and gonads for all treatment groups (excipient+PBS n=8, variant+PBS n=7, excipient+CBE n=7, and variant+CBE n=9). Biodistribution is shown as vector genomes per 1 μg of genomic DNA. Vector genome presence was quantified by quantitative PCR using a vector reference standard curve; genomic DNA concentration was evaluated by A260 optical density measurement. Means are presented. Error bars are SEM. *p<0.05; **p<0.01; ***p<0.001, nominal p-values for treatment groups by linear regression in the CBE-treated animals, with collection days and gender corrected for as covariates.

FIG. 15 shows representative data for in-life assessment of rAAV dose ranging in a CBE mouse model. Mice received excipient or one of three different doses of rAAV-GBA1 by ICV delivery at P3: 3.2 e9 vg, 1.0 e10 vg, or 3.2 e10 vg. At P8, daily IP treatment of 25 mg/kg CBE was initiated. Mice that received excipient and CBE or excipient and PBS served as controls. All treatment groups started with n=10 (5M/5F) per group. All mice were sacrificed one day after their final CBE dose (P38-P40). All treatment groups were weighed daily, and their weight was analyzed at P36. Motor performance was assessed by latency to fall on Rotarod at P24 and latency to traverse the Tapered Beam at P30. Due to early lethality, the number of mice participating in the behavioral assays was: excipient+PBS n=10, excipient+CBE n=9, and 3.2 e9 vg rAAV-GBA1+CBE n=6, 1.0 e10 vg rAAV-GBA1+CBE n=10, 3.2 e10 vg rAAV-GBA1+CBE n=7. Means are presented. Error bars are SEM; * p<0.05; **p<0.01 for nominal p-values by linear regression in the CBE-treated groups, with gender corrected for as a covariate.

FIG. 16 shows representative data for biochemical assessment of rAAV dose ranging in a CBE mouse model. The cortex of all treatment groups (excipient+PBS n=10, excipient+CBE n=9, and 3.2 e9 vg rAAV-GBA1+CBE n=6, 1.0 e10 vg rAAV-GBA1+CBE n=10, 3.2 e10 vg rAAV-GBA1+CBE n=7) was used to measure GCase activity, GluSph levels, GluCer levels, and vector genomes. GCase activity is shown as ng of GCase per mg of total protein. GluSph and GluCer levels are shown as pmol per mg wet weight of the tissue.

Biodistribution is shown as vector genomes per 1 μg of genomic DNA. Vector genome presence was quantified by quantitative PCR using a vector reference standard curve; genomic DNA concentration was evaluated by A260 optical density measurement. Vector genome presence was also measured in the liver (E). Means are presented. Error bars are SEM. **p<0.01; ***p<0.001 for nominal p-values by linear regression in the CBE-treated groups, with gender corrected for as a covariate.

FIG. 17 shows representative data for tapered beam analysis in maximal dose rAAV-GBA1 in a genetic mouse model. Motor performance of the treatment groups (WT+excipient, n=5), 4L/PS-NA+excipient (n=6), and 4L/PS-NA+rAAV-GBA1 (n=5)) was assayed by Beam Walk 4 weeks post rAAV-GBA1 administration. The total slips and active time are shown as total over 5 trials on different beams. Speed and slips per speed are shown as the average over 5 trials on different beams. Means are presented. Error bars are SEM.

FIG. 18 shows representative data for in vitro expression of rAAV constructs encoding progranulin (PGRN) protein (also referred to as GRN protein). The left panel shows a standard curve of progranulin (PGRN) ELISA assay. The bottom panel shows a dose-response of PGRN expression measured by ELISA assay in cell lysates of HEK293T cells transduced with rAAV. MOI=multiplicity of infection (vector genomes per cell).

FIG. 19 shows representative data for in vitro expression of rAAV constructs encoding GBA1 in combination with Prosaposin (PSAP), SCARB2, and/or one or more inhibitory nucleic acids. Data indicate transfection of HEK293 cells with each construct resulted in overexpression of the transgenes of interest relative to mock transfected cells.

FIG. 20 is a schematic depicting an rAAV vectors comprising a “D” region located on the “outside” of the ITR (e.g., proximal to the terminus of the ITR relative to the transgene insert or expression construct) (top) and a wild-type rAAV vectors having ITRs on the “inside” of the vector (e.g., proximal to the transgene insert of the vector).

FIG. 21 a schematic depicting one embodiment of a vector comprising an expression construct encoding GBA2 or a portion thereof, and an interfering RNA for α-Syn.

FIG. 22 is a schematic depicting one embodiment of a vector comprising an expression construct encoding Gcase (e.g., GBA1 or a portion thereof) and Galactosylceramidase (e.g., GALC or a portion thereof). Expression of the coding sequences of Gcase and Galactosylceramidase are separated by a T2A self-cleaving peptide sequence.

FIG. 23 is a schematic depicting one embodiment of a vector comprising an expression construct encoding Gcase (e.g., GBA1 or a portion thereof) and Galactosylceramidase (e.g., GALC or a portion thereof). Expression of the coding sequences of Gcase and Galactosylceramidase are separated by a T2A self-cleaving peptide sequence.

FIG. 24 is a schematic depicting one embodiment of a vector comprising an expression construct encoding Gcase (e.g., GBA1 or a portion thereof), Cathepsin B (e.g., CTSB or a portion thereof), and an interfering RNA for α-Syn. Expression of the coding sequences of Gcase and Cathepsin B are separated by a T2A self-cleaving peptide sequence.

FIG. 25 is a schematic depicting one embodiment of a vector comprising an expression construct encoding Gcase (e.g., GBA1 or a portion thereof), Sphingomyelin phosphodiesterase 1 (e.g., SMPD1 a portion thereof, and an interfering RNA for α-Syn.

FIG. 26 is a schematic depicting one embodiment of a vector comprising an expression construct encoding Gcase (e.g., GBA1 or a portion thereof) and Galactosylceramidase (e.g., GALC or a portion thereof). The coding sequences of Gcase and Galactosylceramidase are separated by an internal ribosomal entry site (IRES).

FIG. 27 is a schematic depicting one embodiment of a vector comprising an expression construct encoding Gcase (e.g., GBA1 or a portion thereof) and Cathepsin B (e.g., CTSB or a portion thereof). Expression of the coding sequences of Gcase and Cathepsin B are each driven by a separate promoter.

FIG. 28 is a schematic depicting one embodiment of a vector comprising an expression construct encoding Gcase (e.g., GBA1 or a portion thereof), GCH1 (e.g., GCH1 or a portion thereof), and an interfering RNA for α-Syn. The coding sequences of Gcase and GCH1 are separated by an T2A self-cleaving peptide sequence

FIG. 29 is a schematic depicting one embodiment of a vector comprising an expression construct encoding Gcase (e.g., GBA1 or a portion thereof), RAB7L1 (e.g., RAB7L1 or a portion thereof), and an interfering RNA for α-Syn. The coding sequences of Gcase and RAB7L1 are separated by an T2A self-cleaving peptide sequence.

FIG. 30 is a schematic depicting one embodiment of a vector comprising an expression construct encoding Gcase (e.g., GBA1 or a portion thereof), GCH1 (e.g., GCH1 or a portion thereof), and an interfering RNA for α-Syn. Expression of the coding sequences of Gcase and GCH1 are an internal ribosomal entry site (IRES).

FIG. 31 is a schematic depicting one embodiment of a vector comprising an expression construct encoding VPS35 (e.g., VPS35 or a portion thereof) and interfering RNAs for α-Syn and TMEM106B.

FIG. 32 is a schematic depicting one embodiment of a vector comprising an expression construct encoding Gcase (e.g., GBA1 or a portion thereof), IL-34 (e.g., IL34 or a portion thereof), and an interfering RNA for α-Syn. The coding sequences of Gcase and IL-34 are separated by T2A self-cleaving peptide sequence.

FIG. 33 is a schematic depicting one embodiment of a vector comprising an expression construct encoding Gcase (e.g., GBA1 or a portion thereof) and IL-34 (e.g., IL34 or a portion thereof). The coding sequences of Gcase and IL-34 are separated by an internal ribosomal entry site (IRES).

FIG. 34 is a schematic depicting one embodiment of a vector comprising an expression construct encoding Gcase (e.g., GBA1 or a portion thereof) and TREM2 (e.g., TREM2 or a portion thereof). Expression of the coding sequences of Gcase and TREM2 are each driven by a separate promoter.

FIG. 35 is a schematic depicting one embodiment of a vector comprising an expression construct encoding Gcase (e.g., GBA1 or a portion thereof) and IL-34 (e.g., IL34 or a portion thereof). Expression of the coding sequences of Gcase and IL-34 are each driven by a separate promoter.

FIGS. 36A-36B show representative data for overexpression of TREM2 and GBA1 in HEK293 cells relative to control transduced cells, as measured by qPCR and ELISA. FIG. 36A shows data for overexpression of TREM2. FIG. 36B shows data for overexpression of GBA1 from the same construct.

FIG. 37 shows representative data indicating successful silencing of SNCA in vitro by GFP reporter assay (top) and α-Syn assay (bottom).

FIG. 38 shows representative data indicating successful silencing of TMEM106B in vitro by GFP reporter assay (top) and α-Syn assay (bottom).

FIG. 39 is a schematic depicting one embodiments of a vector comprising an expression construct encoding PGRN (also referred to as GRN).

FIG. 40 shows data for transduction of HEK293 cells using rAAVs having ITRs with wild-type (circles) or alternative (e.g., “outside”; squares) placement of the “D” sequence. The rAAVs having ITRs placed on the “outside” were able to transduce cells as efficiently as rAAVs having wild-type ITRs.

FIG. 41 is a schematic depicting one embodiment of a vector comprising an expression construct encoding Gcase (e.g., GBA1 or a portion thereof).

FIG. 42 is a schematic depicting one embodiment of a vector comprising an expression construct encoding Gcase (e.g., GBA1 or a portion thereof).

FIG. 43 is a schematic depicting one embodiment of a vector comprising an expression construct encoding Gcase (e.g., GBA1 or a portion thereof) and an interfering RNA for α-Syn.

FIG. 44 is a schematic depicting one embodiments of a vector comprising an expression construct encoding PGRN (also referred to as GRN).

FIG. 45 is a schematic depicting one embodiments of a vector comprising an expression construct encoding PGRN (also referred to as GRN).

FIG. 46 is a schematic depicting one embodiments of a vector comprising an expression construct encoding PGRN (also referred to as GRN) and an interfering RNA for microtubule-associated protein tau (MAPT). The nucleic acid sequence of this vector is set forth in SEQ ID NO: 142.

FIG. 47 is a schematic depicting one embodiment of a vector comprising an expression construct encoding Gcase (e.g., GBA1 or a portion thereof) and an interfering RNA for α-Syn.

FIG. 48 is a schematic depicting one embodiments of a vector comprising an expression construct encoding PSAP.

FIG. 49 is a schematic depicting one embodiment of a vector comprising an expression construct encoding Gcase (e.g., GBA1 or a portion thereof).

FIG. 50 is a schematic depicting one embodiment of a vector comprising an expression construct encoding Gcase (e.g., GBA1 or a portion thereof) and Galactosylceramidase (e.g., GALC or a portion thereof).

FIG. 51 is a schematic depicting one embodiment of a plasmid comprising an rAAV vector that includes an expression construct encoding Gcase (e.g., GBA1 or a portion thereof), Prosaposin (e.g., PSAP or a portion thereof), and an interfering RNA for α-Syn.

FIG. 52 is a schematic depicting one embodiment of a vector comprising an expression construct encoding Gcase (e.g., GBA1 or a portion thereof) and an inhibitory RNA targeting SNCA.

FIG. 53 is a schematic depicting one embodiment of a plasmid comprising an rAAV vector that includes an expression construct encoding SNCA.

FIG. 54 is a schematic depicting one embodiment of a plasmid comprising an rAAV vector that includes an expression construct encoding an inhibitory RNA targeting SNCA. The inhibitory RNA is positioned within an intron between the promoter sequence and the Gcase encoding sequence.

FIG. 55 is a schematic depicting one embodiment of a plasmid comprising an rAAV vector that includes an expression construct encoding progranulin (PGRN, also referred to as GRN) and an inhibitory RNA targeting SNCA. The inhibitory RNA is positioned within an intron between the promoter sequence and the Gcase encoding sequence.

FIG. 56 is a schematic depicting one embodiment of a plasmid comprising an rAAV vector that includes an expression construct encoding Gcase (GBA1) and an inhibitory RNA targeting SNCA. The inhibitory RNA is positioned within an intron between the promoter sequence and the Gcase encoding sequence.

FIG. 57 is a schematic depicting one embodiment of a plasmid comprising an rAAV vector that includes an expression construct encoding Gcase (GBA1) and an inhibitory RNA targeting SNCA. The inhibitory RNA is positioned within an intron between the promoter sequence and the Gcase encoding sequence.

FIG. 58 is a schematic depicting one embodiment of a plasmid comprising an rAAV vector that includes an expression construct encoding Gcase (GBA1) and an inhibitory RNA targeting SNCA. The “D” sequence of the 3′ITR is positioned on the “outside” of the vector.

FIG. 59 is a schematic depicting one embodiment of a plasmid comprising an rAAV vector that includes an expression construct encoding Gcase (GBA1) and an inhibitory RNA targeting SNCA. The inhibitory RNA is positioned within an intron between the promoter sequence and the Gcase encoding sequence.

FIG. 60 is a schematic depicting one embodiment of a plasmid comprising an rAAV vector that includes an expression construct encoding Gcase (GBA1) and an inhibitory RNA targeting SNCA. The inhibitory RNA is positioned within an intron between the promoter sequence and the Gcase encoding sequence.

FIG. 61 is a schematic depicting one embodiment of a plasmid comprising an rAAV vector that includes an expression construct encoding Gcase (GBA1) and an inhibitory RNA targeting SNCA.

FIG. 62 is a schematic depicting one embodiment of a plasmid comprising an rAAV vector that includes an expression construct encoding Gcase (GBA1) and an inhibitory RNA targeting SNCA. The inhibitory RNA is positioned within an intron between the promoter sequence and the Gcase encoding sequence.

FIG. 63 is a schematic depicting one embodiment of a plasmid comprising an rAAV vector that includes an expression construct encoding Gcase (GBA1) and an inhibitory RNA targeting SNCA. The inhibitory RNA is positioned within an intron between the promoter sequence and the Gcase encoding sequence.

FIG. 64 is a schematic depicting one embodiment of a plasmid comprising an rAAV vector that includes an expression construct encoding Gcase (GBA1) and progranulin (PGRN, also referred to as GRN), and an inhibitory RNA targeting TMEM106B. The inhibitory RNA is positioned within an intron between the promoter sequence and the Gcase encoding sequence.

FIG. 65 is a schematic depicting one embodiment of a plasmid comprising an rAAV vector that includes an expression construct encoding an inhibitory RNA targeting RPS25.

FIG. 66 is a schematic depicting one embodiment of a plasmid comprising an rAAV vector that includes an expression construct encoding an inhibitory RNA targeting RPS25.

FIG. 67 is a schematic depicting one embodiment of a plasmid comprising an rAAV vector that includes an expression construct encoding an inhibitory RNA targeting MAPT.

FIG. 68 is a schematic depicting one embodiment of a plasmid comprising an rAAV vector that includes an expression construct encoding an inhibitory RNA targeting MAPT.

FIG. 69 is a schematic depicting one embodiment of a plasmid comprising an rAAV vector that includes an expression construct encoding progranulin (PGRN, also referred to as GRN) and an inhibitory RNA targeting MAPT. The inhibitory RNA is positioned within an intron between the promoter sequence and the PGRN (also referred to as GRN) encoding sequence.

FIG. 70 is a schematic depicting one embodiment of a plasmid comprising an rAAV vector that includes an expression construct encoding an inhibitory RNA targeting MAPT.

FIG. 71 is a schematic depicting one embodiment of a plasmid comprising an rAAV vector that includes an expression construct encoding progranulin (PGRN, also referred to as GRN) and an inhibitory RNA targeting MAPT. The inhibitory RNA is positioned within an intron between the promoter sequence and the PGRN (also referred to as GRN) encoding sequence.

FIG. 72 is a schematic depicting one embodiment of a vector comprising an expression construct encoding Gcase (e.g., GBA1 or a portion thereof) and an inhibitory RNA targeting SNCA. Nucleic acid sequence of this vector is set forth in SEQ ID NO: 141.

FIG. 73 is a schematic depicting one embodiment of a vector comprising an expression construct encoding Gcase (e.g., GBA1 or a portion thereof) and an inhibitory RNA targeting SNCA. Nucleic acid sequence of this vector is set forth in SEQ ID NO: 143.

FIG. 74 is a schematic depicting one embodiment of a plasmid comprising an rAAV vector that includes an expression construct encoding Gcase (GBA1) and prosaposin (PSAP), and an inhibitory RNA targeting SNCA. Nucleic acid sequence of this vector is set forth in SEQ ID NO: 144.

FIG. 75A-75C are charts showing MAPT knockdown in SY5Y Cells by RNA interference. FIG. 75A shows that immunofluorescent stationing of the AAV vectors using a probe directed to BGHpA. FIG. 75B shows RT-PCR results of MAPT expression 3 and 7 days post transduction. FIG. 75C shows the general information of the rAAV virus stocks used for transduction.

### DETAILED DESCRIPTION

The disclosure is based, in part, on compositions and methods for expression of combinations of certain gene products (e.g., gene products associated with CNS disease) in a subject. A gene product can be a protein, a fragment (e.g., portion) of a protein, an interfering nucleic acid that inhibits a CNS disease-associated gene, etc. In some embodiments, a gene product is a protein or a protein fragment encoded by a CNS disease-associated gene. In some embodiments, a gene product is an interfering nucleic acid (e.g., shRNA, siRNA, miRNA, amiRNA, etc.) that inhibits a CNS disease-associated gene.

A CNS disease-associated gene refers to a gene encoding a gene product that is genetically, biochemically or functionally associated with a CNS disease, such as PD. For example, individuals having mutations in the GBA1 gene (which encodes the protein Gcase), have been observed to be have an increased risk of developing PD compared to individuals that do not have a mutation in GBA1. In another example, synucleinopathies (e.g., PD, etc.) are associated with accumulation of protein aggregates comprising α-Synuclein (α-Syn) protein; accordingly, SNCA (which encodes α-Syn) is a PD-associated gene. In some embodiments, an expression cassette described herein encodes a wild-type or non-mutant form of a CNS disease-associated gene, for example a PD-associated gene (or coding sequence thereof). Examples of CNS diseases-associated genes (e.g., PD-associated genes, AD-associated genes, FTD-associated genes, etc.) are listed in Table 1.

TABLE 1
Examples of CNS disease-associated genes and gene products

| | | | NCBI Accession |
|---|---|---|---|
| Name | Gene | Function | No. |
| Lysosome membrane protein 2 | SCARB2/LIMP2 | lysosomal receptor for | NP_005497.1 |
| | | glucosylceramidase | (Isoform 1), |
| | | (GBA targeting) | NP_001191184.1 |
| | | | (Isoform 2) |
| Prosaposin | PSAP | precursor for saposins | AAH01503.1, |
| | | A, B, C, and D, which | AAH07612.1, |
| | | localize to the lysosomal | AAH04275.1, |
| | | compartment and | AAA60303.1 |
| | | facilitate the catabolism | |
| | | of glycosphingolipids | |
| | | with short | |
| | | oligosaccharide groups | |
| beta-Glucocerebrosidase | GBA1 | cleaves the beta- | NP_001005742.1 |
| | | glucosidic linkage of | (Isoform 1), |
| | | glucocerebroside | NP_001165282.1 |
| | | | (Isoform 2), |
| | | | NP_001165283.1 |
| | | | (Isoform 3) |
| Non-lysosomal | GBA2 | catalyzes the conversion | NP_065995.1 |
| Glucosylceramidase | | of glucosylceramide to | (Isoform 1), |
| | | free glucose and ceramide | NP_001317589.1 |
| | | | (Isoform 2) |
| Galactosylceramidase | GALC | removes galactose from | EAW81359.1 |
| | | ceramide derivatives | (Isoform CRA_a), |
| | | | EAW81360.1 |
| | | | (Isoform CRA_b), |
| | | | EAW81362.1 |
| | | | (Isoform CRA_c) |
| Sphingomyelin | SMPD1 | converts sphingomyelin | EAW68726.1 |
| phosphodiesterase 1 | | to ceramide | (Isoform CRA_a), |
| | | | EAW68727.1 |
| | | | (Isoform CRA_b), |
| | | | EAW68728.1 |
| | | | (Isoform CRA_c), |
| | | | EAW68729.1 |
| | | | (Isoform CRA_d) |
| Cathepsin B | CTSB | thiol protease believed | AAC37547.1, |
| | | to participate in | AAH95408.1, |
| | | intracellular degradation | AAH10240.1 |
| | | and turnover of proteins; | |
| | | also implicated in tumor | |
| | | invasion and metastasis | |
| RAB7, member RAS oncogene | RAB7L1 | regulates vesicular transport | AAH02585.1 |
| family-like 1 | | | |
| Vacuolar protein sorting- | VPS35 | component of retromer | NP_060676.2 |
| associated protein 35 | | cargo-selective complex | |
| GTP cyclohydrolase 1 | GCH1 | responsible for | AAH25415.1 |
| | | hydrolysis of guanosine | |
| | | triphosphate to form | |
| | | 7.8-dihydroneopterin | |
| | | triphosphate | |
| Interleukin 34 | IL34 | increases growth or | AAH29804.1 |
| | | survival of monocytes; | |
| | | elicits activity by | |
| | | binding the Colony | |
| | | stimulating factor 1 | |
| | | receptor | |
| Triggering receptor expressed on | TREM2 | forms a receptor | AAF69824.1 |
| myeloid cells 2 | | signaling complex with | |
| | | the TYRO protein | |
| | | tyrosine kinase binding | |
| | | protein; functions in | |
| | | immune response and | |
| | | may be involved in | |
| | | chronic inflammation | |
| Progranulin | PGRN (also referred to as | plays a role in | NP_002087.1 |
| | GRN) | development, | |
| | | inflammation, cell | |
| | | proliferation and protein | |
| | | homeostasis | |
| alpha-Synuclein | SNCA | plays a role in | NP_001139527.1 |
| | | maintaining a supply of | |
| | | synaptic vesicles in | |
| | | presynaptic terminals by | |
| | | clustering synaptic vesicles, | |
| | | and may help regulate | |
| | | the release of dopamine | |
| Transmembrane protein 106B | TMEM106B | plays a role in dendrite | NP_060844.2 |
| | | morphogenesis and | |
| | | regulation of lysosomal | |
| | | trafficking | |
| Microtubule associated protein | MAPT | plays a role in | NP_005901.2 |
| tau | | maintaining stability of | |
| | | microtubules in axons | |

### Isolated Nucleic Acids and Vectors

An isolated nucleic acid may be DNA or RNA. As used herein, the term “isolated” means artificially produced. An “isolated nucleic acid”, as used herein, refers to nucleic acids (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable by recombinant DNA techniques well known in the art.

The disclosure provides, in some aspects, an isolated nucleic acids (e.g., rAAV vectors) comprising an expression construct encoding one or more CNS disease-associated genes (e.g., PD-associated genes), for example a Gcase, a Prosaposin, a LIMP2/SCARB2, a GBA2, GALC protein, a CTSB protein, a SMPD1, a GCH1 protein, a RAB7L protein, a VPS35 protein, a IL-34 protein, a TREM2 protein, or a TMEM106B protein. The disclosure also provides, in some aspects, isolated nucleic acids (e.g., rAAV vectors) encoding one or more inhibitory nucleic acids that target one or more CNS disease-associated gene, for example SNCA, TMEM106B, RPS25, and MAPT. In some embodiments, the isolated nucleic acid encoding the CNS disease-associated genes may further comprises coding sequences for inhibitory nucleic acids targeting one or more CNS disease-associated genes. In some embodiments, the CNS disease-associated genes and the inhibitory nucleic acids targeting CNS disease-associated genes are encoded on different nucleic acids.

In some aspects, the disclosure provides an isolated nucleic acid comprising an expression construct encoding Gcase (e.g., the gene product of GBA1 gene). Gcase, also referred to as β-glucocerebrosidase or GBA, refers to a lysosomal protein that cleaves the beta-glucosidic linkage of the chemical glucocerebroside, an intermediate in glycolipid metabolism. In humans, Gcase is encoded by the GBA1 gene, located on chromosome 1. In some embodiments, GBA1 encodes a peptide that is represented by NCBI Reference Sequence NCBI Reference Sequence NP_000148.2 (SEQ ID NO: 14). In some embodiments, an isolated nucleic acid comprises a Gcase-encoding sequence that has been codon optimized (e.g., codon optimized for expression in mammalian cells, for example human cells), such as the sequence set forth in SEQ ID NO: 15.

In some aspects, the disclosure provides an isolated nucleic acid comprising an expression construct encoding Prosaposin (e.g., the gene product of PSAP gene). Prosaposin is a precursor glycoprotein for sphingolipid activator proteins (saposins) A, B, C, and D, which facilitate the catabolism of glycosphingolipids with short oligosaccharide groups. In humans, the PSAP gene is located on chromosome 10. In some embodiments, PSAP encodes a peptide that is represented by NCBI Reference Sequence NP_002769.1 (e.g., SEQ ID NO: 16). In some embodiments, an isolated nucleic acid comprises a prosaposin-encoding sequence that has been codon optimized (e.g., codon optimized for expression in mammalian cells, for example human cells), such as the sequence set forth in SEQ ID NO: 17.

Aspects of the disclosure relate to an isolated nucleic acid comprising an expression construct encoding LIMP2/SCARB2 (e.g., the gene product of SCARB2 gene). SCARB2 refers to a membrane protein that regulates lysosomal and endosomal transport within a cell. In humans, SCARB2 gene is located on chromosome 4. In some embodiments, the SCARB2 gene encodes a peptide that is represented by NCBI Reference Sequence NP_005497.1 (SEQ ID NO: 18). In some embodiments, an isolated nucleic acid comprises the sequence set forth in SEQ ID NO: 19. In some embodiments the isolated nucleic acid comprises a SCARB2-encoding sequence that has been codon optimized.

Aspects of the disclosure relate to an isolated nucleic acid comprising an expression construct encoding GBA2 protein (e.g., the gene product of GBA2 gene). GBA2 protein refers to non-lysosomal glucosylceramidase. In humans, GBA2 gene is located on chromosome 9. In some embodiments, the GBA2 gene encodes a peptide that is represented by NCBI Reference Sequence NP_065995.1 (SEQ ID NO: 30). In some embodiments, an isolated nucleic acid comprises the sequence set forth in SEQ ID NO: 31. In some embodiments the isolated nucleic acid comprises a GBA2-encoding sequence that has been codon optimized.

Aspects of the disclosure relate to an isolated nucleic acid comprising an expression construct encoding GALC protein (e.g., the gene product of GALC gene). GALC protein refers to galactosylceramidase (or galactocerebrosidase), which is an enzyme that hydrolyzes galactose ester bonds of galactocerebroside, galactosylsphingosine, lactosylceramide, and monogalactosyldiglyceride. In humans, GALC gene is located on chromosome 14. In some embodiments, the GALC gene encodes a peptide that is represented by NCBI Reference Sequence NP_000144.2 (SEQ ID NO: 33). In some embodiments, an isolated nucleic acid comprises the sequence set forth in SEQ ID NO: 34. In some embodiments the isolated nucleic acid comprises a GALC-encoding sequence that has been codon optimized.

Aspects of the disclosure relate to an isolated nucleic acid comprising an expression construct encoding CTSB protein (e.g., the gene product of CTSB gene). CTSB protein refers to cathepsin B, which is a lysosomal cysteine protease that plays an important role in intracellular proteolysis. In humans, CTSB gene is located on chromosome 8. In some embodiments, the CTSB gene encodes a peptide that is represented by NCBI Reference Sequence NP_001899.1 (SEQ ID NO: 35). In some embodiments, an isolated nucleic acid comprises the sequence set forth in SEQ ID NO: 36. In some embodiments the isolated nucleic acid comprises a CTSB-encoding sequence that has been codon optimized.

Aspects of the disclosure relate to an isolated nucleic acid comprising an expression construct encoding SMPD1 protein (e.g., the gene product of SMPD1 gene). SMPD1 protein refers to sphingomyelin phosphodiesterase 1, which is a hydrolase enzyme that is involved in sphingolipid metabolism. In humans, SMPD1 gene is located on chromosome 11. In some embodiments, the SMPD1 gene encodes a peptide that is represented by NCBI Reference Sequence NP_000534.3 (SEQ ID NO: 37). In some embodiments, an isolated nucleic acid comprises the sequence set forth in SEQ ID NO: 38. In some embodiments the isolated nucleic acid comprises a SMPD1-encoding sequence that has been codon optimized.

Aspects of the disclosure relate to an isolated nucleic acid comprising an expression construct encoding GCH1 protein (e.g., the gene product of GCH1 gene). GCH1 protein refers to GTP cyclohydrolase I, which is a hydrolase enzyme that is part of the folate and biopterin biosynthesis pathways. In humans, GCH1 gene is located on chromosome 14. In some embodiments, the GCH1 gene encodes a peptide that is represented by NCBI Reference Sequence NP_000152.1 (SEQ ID NO: 45). In some embodiments, an isolated nucleic acid comprises the sequence set forth in SEQ ID NO: 46. In some embodiments the isolated nucleic acid comprises a GCH1-encoding sequence that has been codon optimized.

Aspects of the disclosure relate to an isolated nucleic acid comprising an expression construct encoding RAB7L protein (e.g., the gene product of RAB7L gene). RAB7L protein refers to RAB7, member RAS oncogene family-like 1, which is a GTP binding protein. In humans, RAB7L gene is located on chromosome 1. In some embodiments, the RAB7L gene encodes a peptide that is represented by NCBI Reference Sequence NP_003920.1 (SEQ ID NO: 47). In some embodiments, an isolated nucleic acid comprises the sequence set forth in SEQ ID NO: 48. In some embodiments the isolated nucleic acid comprises a RAB7L-encoding sequence that has been codon optimized.

Aspects of the disclosure relate to an isolated nucleic acid comprising an expression construct encoding VPS35 protein (e.g., the gene product of VPS35 gene). VPS35 protein refers to vacuolar protein sorting-associated protein 35, which is part of a protein complex involved in retrograde transport of proteins from endosomes to the trans-Golgi network. In humans, VPS35 gene is located on chromosome 16. In some embodiments, the VPS35 gene encodes a peptide that is represented by NCBI Reference Sequence NP_060676.2 (SEQ ID NO: 49). In some embodiments, an isolated nucleic acid comprises the sequence set forth in SEQ ID NO: 50. In some embodiments the isolated nucleic acid comprises a VPS35-encoding sequence that has been codon optimized.

Aspects of the disclosure relate to an isolated nucleic acid comprising an expression construct encoding IL-34 protein (e.g., the gene product of IL34 gene). IL-34 protein refers to interleukin 34, which is a cytokine that increases growth and survival of monocytes. In humans, IL34 gene is located on chromosome 16. In some embodiments, the IL34 gene encodes a peptide that is represented by NCBI Reference Sequence NP_689669.2 (SEQ ID NO: 55). In some embodiments, an isolated nucleic acid comprises the sequence set forth in SEQ ID NO: 56. In some embodiments the isolated nucleic acid comprises a IL-34-encoding sequence that has been codon optimized.

Aspects of the disclosure relate to an isolated nucleic acid comprising an expression construct encoding TREM2 protein (e.g., the gene product of TREM2 gene). TREM2 protein refers to triggering receptor expressed on myeloid cells 2, which is an immunoglobulin superfamily receptor found on myeloid cells. In humans, TREM2 gene is located on chromosome 6. In some embodiments, the TREM2 gene encodes a peptide that is represented by NCBI Reference Sequence NP_061838.1 (SEQ ID NO: 57). In some embodiments, the isolated nucleic acid comprises the sequence set forth in SEQ ID NO: 58. In some embodiments an isolated nucleic acid comprises a TREM2-encoding sequence that has been codon optimized.

Aspects of the disclosure relate to an isolated nucleic acid comprising an expression construct encoding TMEM106B protein (e.g., the gene product of TMEM106B gene). TMEM106B protein refers to transmembrane protein 106B, which is a protein involved in dendrite morphogenesis and regulation of lysosomal trafficking. In humans, TMEM106B gene is located on chromosome 7. In some embodiments, the TMEM106B gene encodes a peptide that is represented by NCBI Reference Sequence NP_060844.2 (SEQ ID NO: 63). In some embodiments, an isolated nucleic acid comprises the sequence set forth in SEQ ID NO: 64. In some embodiments the isolated nucleic acid comprises a TMEM106B-encoding sequence that has been codon optimized.

Aspects of the disclosure relate to an isolated nucleic acid comprising an expression construct encoding progranulin protein (e.g., the gene product of GRN gene). PGRN protein refers to progranulin, which is a protein involved in development, inflammation, cell proliferation and protein homeostasis. In humans, PGRN (also referred to as GRN) gene is located on chromosome 17. In some embodiments, the GRN gene encodes a peptide that is represented by NCBI Reference Sequence NP_002078.1 (SEQ ID NO: 67). In some embodiments, an isolated nucleic acid comprises the sequence set forth in SEQ ID NO: 68. In some embodiments the isolated nucleic acid comprises a PGRN-encoding sequence (GRN-encoding sequence) that has been codon optimized.

In some aspects, the disclosure provides an isolated nucleic acid comprising an expression construct encoding a first gene product and a second gene product, wherein each gene product independently is selected from the gene products, or portions thereof, set forth in Table 1 or an inhibitory nucleic acid targeting a gene or gene product set forth in Table 1.

In some embodiments, a gene product is encoded by a coding portion (e.g., a cDNA) of a naturally occurring gene. In some embodiments, a first gene product is a protein (or a fragment thereof) encoded by the GBA1 gene. In some embodiments, a gene product is a protein (or a fragment thereof) encoded by another gene listed in Table 1, for example the SCARB2/LIMP2 gene or the PSAP gene. However, the skilled artisan recognizes that the order of expression of a first gene product (e.g., Gcase) and a second gene product (e.g., LIMP2, etc.) can generally be reversed (e.g., LIMP2 is the first gene product and Gcase is the second gene product). In some embodiments, a gene product is a fragment (e.g., portion) of a gene listed in Table 1. A protein fragment may comprise about 50%, about 60%, about 70%, about 80% about 90% or about 99% of a protein encoded by the genes listed in Table 1. In some embodiments, a protein fragment comprises between 50% and 99.9% (e.g., any value between 50% and 99.9%) of a protein encoded by a gene listed in Table 1.

Pathologically, disorders such as PD and Gaucher disease are associated with accumulation of protein aggregates composed largely of α-Synuclein (α-Syn) protein. Accordingly, in some embodiments, isolated nucleic acids described herein comprise an inhibitory nucleic acid that reduces or prevents expression of α-Syn protein.

In some aspects, the disclosure provides an isolated nucleic acid comprising an expression construct encoding one or more interfering nucleic acids (e.g., dsRNA, siRNA, miRNA, amiRNA, etc.) that target an microtubule-associated protein tau, MAPT (e.g., the gene product of MAPT gene), which is involved in Alzheimer's disease and FTD-tau.

Generally, an isolated nucleic acid as described herein may encode 1, 2, 3, 4, 5, 6, 7, 8, 9, 10, or more inhibitory nucleic acids (e.g., dsRNA, siRNA, shRNA, miRNA, amiRNA, etc.). In some embodiments, an isolated nucleic acid encodes more than 10 inhibitory nucleic acids. In some embodiments, each of the one or more inhibitory nucleic acids targets a different gene or a portion of a gene (e.g., a first miRNA targets a first target sequence of a gene and a second miRNA targets a second target sequence of the gene that is different than the first target sequence). In some embodiments, each of the one or more inhibitory nucleic acids targets the same target sequence of the same gene (e.g., an isolated nucleic acid encodes multiple copies of the same miRNA).

In some aspects, the disclosure provides relate to an isolated nucleic acid comprising an expression construct encoding one or more interfering nucleic acids (e.g., dsRNA, siRNA, miRNA, amiRNA, etc.) that target an α-Synuclein protein (e.g., the gene product of SNCA gene). α-Synuclein protein refers to a protein found in brain tissue, which is plays a role in maintaining a supply of synaptic vesicles in presynaptic terminals by clustering synaptic vesicles and regulating the release of dopamine. In humans, SNCA gene is located on chromosome 4. In some embodiments, the SNCA gene encodes a peptide that is represented by NCBI Reference Sequence NP_001139527.1. In some embodiments, a SNCA gene comprises the sequence set forth in SEQ ID NO: 90.

An inhibitory nucleic acid targeting SNCA may comprise a region of complementarity (e.g., a region of the inhibitory nucleic acid that hybridizes to the target gene, such as SNCA) that is between 6 and 50 nucleotides in length. In some embodiments, an inhibitory nucleic acid comprises a region of complementarity with SNCA that is between about 6 and 30, about 8 and 20, or about 10 and 19 nucleotides in length. In some embodiments, an inhibitory nucleic acid is complementary with at least 3, 4, 5, 6, 7, 8, 9, 10, 11, 12, 13, 14, 15, 16, 17, 18, 19, 20, 21, 22, 23, 24, or 25 contiguous nucleotides of a SNCA sequence.

In some aspects, the disclosure provides an isolated nucleic acid comprising an expression construct encoding one or more interfering nucleic acids (e.g., dsRNA, siRNA, miRNA, amiRNA, etc.) that target an TMEM106B protein (e.g., the gene product of TMEM106B gene). TMEM106B protein refers to transmembrane protein 106B, which is a protein involved in dendrite morphogenesis and regulation of lysosomal trafficking. In humans, TMEM106B gene is located on chromosome 7. In some embodiments, the TMEM106B gene encodes a peptide that is represented by NCBI Reference Sequence NP_060844.2. In some embodiments, a TMEM106B gene comprises the sequence set forth in SEQ ID NO: 91.

An inhibitory nucleic acid targeting TMEM106B may comprise a region of complementarity (e.g., a region of the inhibitory nucleic acid that hybridizes to the target gene, such as TMEM106B) that is between 6 and 50 nucleotides in length. In some embodiments, an inhibitory nucleic acid comprises a region of complementarity with TMEM106B that is between about 6 and 30, about 8 and 20, or about 10 and 19 nucleotides in length. In some embodiments, an inhibitory nucleic acid is complementary with at least 3, 4, 5, 6, 7, 8, 9, 10, 11, 12, 13, 14, 15, 16, 17, 18, 19, 20, 21, 22, 23, 24, or 25 contiguous nucleotides of a TMEM106B sequence.

In some aspects, the disclosure provides an isolated nucleic acid comprising an expression construct encoding one or more interfering nucleic acids (e.g., dsRNA, siRNA, miRNA, amiRNA, etc.) that target an ribosomal protein s25 (RPS25) (e.g., the gene product of RPS25). RPS25 protein refers to a ribosomal protein which is a subunit of the s40 ribosome, a protein complex involved in protein synthesis. In humans, RPS25 gene is located on chromosome 11. In some embodiments, the RPS25 gene encodes a peptide that is represented by NCBI Reference Sequence NP_001019.1. In some embodiments, a RPS25 gene comprises the sequence set forth in SEQ ID NO: 113.

An inhibitory nucleic acid targeting RPS25 may comprise a region of complementarity (e.g., a region of the inhibitory nucleic acid that hybridizes to the target gene, such as RPS25) that is between 6 and 50 nucleotides in length. In some embodiments, an inhibitory nucleic acid comprises a region of complementarity with RPS25 that is between about 6 and 30, about 8 and 20, or about 10 and 19 nucleotides in length. In some embodiments, an inhibitory nucleic acid is complementary with at least 3, 4, 5, 6, 7, 8, 9, 10, 11, 12, 13, 14, 15, 16, 17, 18, 19, 20, 21, 22, 23, 24, or 25 contiguous nucleotides of a RPS25 sequence.

In some aspects, the disclosure provides an isolated nucleic acid comprising an expression construct encoding one or more interfering nucleic acids (e.g., dsRNA, siRNA, miRNA, amiRNA, etc.) that target an microtubule-associated protein tau, MAPT (e.g., the gene product of MAPT gene). MAPT protein refers to microtubule-associated protein tau, which is a protein involved in microtubule stabilization. In humans, MAPT gene is located on chromosome 17. In some embodiments, the MAPT gene encodes a peptide that is represented by NCBI Reference Sequence NP_005901.2. In some embodiments, a MAPT gene comprises the sequence set forth in SEQ ID NO: 114.

An inhibitory nucleic acid targeting MAPT may comprise a region of complementarity (e.g., a region of the inhibitory nucleic acid that hybridizes to the target gene, such as MAPT) that is between 6 and 50 nucleotides in length. In some embodiments, an inhibitory nucleic acid comprises a region of complementarity with MAPT that is between about 6 and 30, about 8 and 20, or about 10 and 19 nucleotides in length. In some embodiments, an inhibitory nucleic acid is complementary with at least 3, 4, 5, 6, 7, 8, 9, 10, 11, 12, 13, 14, 15, 16, 17, 18, 19, 20, 21, 22, 23, 24, or 25 contiguous nucleotides of a MAPT sequence.

Aspects of the disclosure relate to isolated nucleic acids encoding one or more gene products (e.g., 1, 2, 3, 4, 5, 6, 7, 8, 9, 10, or more gene products). In some embodiments, the one or more gene products are two or more proteins. In some embodiments, the one or more gene products are two or more inhibitory nucleic acids. In some embodiments, the one or more gene products are one or more protein and one or more inhibitory nucleic acid. In some aspects, the disclosure provides an isolated nucleic acid comprising an expression construct encoding a first gene product and a second gene product, wherein each gene product independently is selected from the gene products, or portions thereof, set forth in Table 1 or an inhibitory nucleic acid targeting a gene or gene product set forth in Table 1. A sequence encoding an inhibitory nucleic acid may be placed in an untranslated region (e.g., intron, 5′UTR, 3′UTR, etc.) of the expression vector.

In some embodiments, a gene product is encoded by a coding portion (e.g., a cDNA) of a naturally occurring gene. In some embodiments, a first gene product is a protein (or a fragment thereof) encoded by the GBA1 gene. In some embodiments, a gene product is an inhibitory nucleic acid that targets (e.g., hybridizes to, or comprises a region of complementarity with) a PD-associated gene (e.g., SNCA). A skilled artisan recognizes that the order of expression of a first gene product (e.g., Gcase) and a second gene product (e.g., inhibitory RNA targeting SNCA) can generally be reversed (e.g., the inhibitory RNA is the first gene product and Gcase is the second gene product). In some embodiments, a gene product is a fragment (e.g., portion) of a gene listed in Table 1. A protein fragment may comprise about 50%, about 60%, about 70%, about 80% about 90% or about 99% of a protein encoded by the genes listed in Table 1. In some embodiments, a protein fragment comprises between 50% and 99.9% (e.g., any value between 50% and 99.9%) of a protein encoded by a gene listed in Table 1. In some embodiments, a gene product (e.g., an inhibitory RNA) hybridizes to portion of a target gene (e.g., is complementary to 5, 6, 7, 8, 9, 10, 11, 12, 13, 14, 15, 16, 17, 18, 19, 20, 21, or more contiguous nucleotides of a target gene, for example SNCA). In some embodiments, an expression construct is monocistronic (e.g., the expression construct encodes a single fusion protein comprising a first gene product and a second gene product). In some embodiments, an expression construct is polycistronic (e.g., the expression construct encodes two distinct gene products, for example two different proteins or protein fragments).

A polycistronic expression vector may comprise a one or more (e.g., 1, 2, 3, 4, 5, or more) promoters. Any suitable promoter can be used, for example, a constitutive promoter, an inducible promoter, an endogenous promoter, a tissue-specific promoter (e.g., a CNS-specific promoter), etc. In some embodiments, a promoter is a chicken beta-actin promoter (CBA promoter), a CAG promoter (for example as described by Alexopoulou et al. (2008) BMC Cell Biol. 9:2; doi: 10.1186/1471-2121-9-2), a CD68 promoter, or a JeT promoter (for example as described by Tornøe et al. (2002) Gene 297(1-2):21-32). In some embodiments, a promoter is operably-linked to a nucleic acid sequence encoding a first gene product, a second gene product, or a first gene product and a second gene product. In some embodiments, an expression cassette comprises one or more additional regulatory sequences, including but not limited to transcription factor binding sequences, intron splice sites, poly(A) addition sites, enhancer sequences, repressor binding sites, or any combination of the foregoing.

In some embodiments, a nucleic acid sequence encoding a first gene product and a nucleic acid sequence encoding a second gene product are separated by a nucleic acid sequence encoding an internal ribosomal entry site (IRES). Examples of IRES sites are described, for example, by Mokrejs et al. (2006) Nucleic Acids Res. 34 (Database issue):D125-30. In some embodiments, a nucleic acid sequence encoding a first gene product and a nucleic acid sequence encoding a second gene product are separated by a nucleic acid sequence encoding a self-cleaving peptide. Examples of self-cleaving peptides include but are not limited to T2A, P2A, E2A, F2A, BmCPV 2A, and BmIFV 2A, and those described by Liu et al. (2017) Sci Rep. 7: 2193. In some embodiments, the self-cleaving peptide is a T2A peptide.

In some embodiments, an inhibitory nucleic acid is positioned in an intron of an expression construct, for example in an intron upstream of the sequence encoding a first gene product. An inhibitory nucleic acid can be a double stranded RNA (dsRNA), siRNA, micro RNA (miRNA), artificial miRNA (amiRNA), or an RNA aptamer. Generally, an inhibitory nucleic acid binds to (e.g., hybridizes with) between about 6 and about 30 (e.g., any integer between 6 and 30, inclusive) contiguous nucleotides of a target RNA (e.g., mRNA). In some embodiments, the inhibitory nucleic acid molecule is an miRNA or an amiRNA, for example an miRNA that targets SNCA (the gene encoding α-Syn protein) or TMEM106B (e.g., the gene encoding TMEM106B protein). In some embodiments, the miRNA does not comprise any mismatches with the region of SNCA mRNA to which it hybridizes (e.g., the miRNA is “perfected”). In some embodiments, the inhibitory nucleic acid is an shRNA (e.g., an shRNA targeting SNCA or TMEM106B). In some embodiments, an inhibitory nucleic acid is an artificial miRNA (amiRNA) that includes a miR-155 scaffold and a SNCA or TMEM106B targeting sequence.

In some embodiments, an inhibitory nucleic acid is an artificial microRNA (amiRNA). A microRNA (miRNA) typically refers to a small, non-coding RNA found in plants and animals and functions in transcriptional and post-translational regulation of gene expression. MiRNAs are transcribed by RNA polymerase to form a hairpin-loop structure referred to as a pri-miRNAs which are subsequently processed by enzymes (e.g., Drosha, Pasha, spliceosome, etc.) to for a pre-miRNA hairpin structure which is then processed by Dicer to form a miRNA/miRNA* duplex (where * indicates the passenger strand of the miRNA duplex), one strand of which is then incorporated into an RNA-induced silencing complex (RISC). In some embodiments, an inhibitory RNA as described herein is a miRNA targeting SNCA or TMEM106B.

In some embodiments, an inhibitory nucleic acid targeting SNCA comprises a miRNA/miRNA* duplex. In some embodiments, the miRNA strand of a miRNA/miRNA* duplex comprises or consists of the sequence set forth in any one of SEQ ID NOs: 20-25. In some embodiments, the miRNA* strand of a miRNA/miRNA* duplex comprises or consists of the sequence set forth in any one of SEQ ID NOs: 20-25.

In some embodiments, an inhibitory nucleic acid targeting TMEM106B comprises a miRNA/miRNA* duplex. In some embodiments, the miRNA strand of a miRNA/miRNA* duplex comprises or consists of the sequence set forth in SEQ ID NO: 92 or 93. In some embodiments, the miRNA* strand of a miRNA/miRNA* duplex comprises or consists of the sequence set forth in SEQ ID NOs: 92 or 93.

An artificial microRNA (amiRNA) is derived by modifying native miRNA to replace natural targeting regions of pre-mRNA with a targeting region of interest. For example, a naturally occurring, expressed miRNA can be used as a scaffold or backbone (e.g., a pri-miRNA scaffold), with the stem sequence replaced by that of an miRNA targeting a gene of interest. An artificial precursor microRNA (pre-amiRNA) is normally processed such that one single stable small RNA is preferentially generated. In some embodiments, scAAV vectors and scAAVs described herein comprise a nucleic acid encoding an amiRNA. In some embodiments, the pri-miRNA scaffold of the amiRNA is derived from a pri-miRNA selected from the group consisting of pri-MIR-21, pri-MIR-22, pri-MIR-26a, pri-MIR-30a, pri-MIR-33, pri-MIR-122, pri-MIR-375, pri-MIR-199, pri-MIR-99, pri-MIR-194, pri-MIR-155, and pri-MIR-451. In some embodiments, an amiRNA comprises a nucleic acid sequence targeting SNCA or TMEM106B and an eSIBR amiRNA scaffold, for example as described in Fowler et al. Nucleic Acids Res. 2016 Mar. 18; 44(5): e48.

In some embodiments, an amiRNA targeting SNCA comprises or consists of the sequence set forth in any one of SEQ ID NOs: 94-99. In some embodiments, an amiRNA targeting TMEM106B comprises or consists of the sequence set forth in SEQ ID NOs: 65-66. In some embodiments, an amiRNA targeting RPS25 comprises or consists of the sequence set forth in SEQ ID NOs: 115 to 122. In some embodiments, an amiRNA targeting MAPT comprises or consists of the sequence set forth in SEQ ID NOs: 123-138.

In some embodiments, an isolated nucleic acid or vector (e.g., rAAV vector) described by the disclosure comprises or consists of a sequence set forth in any one of SEQ ID NOs: 1-13, 15, 17, 19-29, 31, 32, 34, 36, 38-44, 46, 48, 50-54, 56, 58-62, 64-66, and 68-145. In some embodiments, an isolated nucleic acid or vector (e.g., rAAV vector) described by the disclosure comprises or consists of a sequence that is complementary (e.g., the complement of) a sequence set forth in any one of SEQ ID NOs: 1-13, 15, 17, 19-29, 31, 32, 34, 36, 38-44, 46, 48, 50-54, 56, 58-62, 64-66, and 68-145. In some embodiments, an isolated nucleic acid or vector (e.g., rAAV vector) described by the disclosure comprises or consists of a sequence that is a reverse complement of a sequence set forth in any one of SEQ ID NOs: 1-13, 15, 17, 19-29, 31, 32, 34, 36, 38-44, 46, 48, 50-54, 56, 58-62, 64-66, and 68-145. In some embodiments, an isolated nucleic acid or vector (e.g., rAAV vector) described by the disclosure comprises or consists of a portion of a sequence set forth in any one of SEQ ID NOs: 1-13, 15, 17, 19-29, 31, 32, 34, 36, 38-44, 46, 48, 50-54, 56, 58-62, 64-66, and 68-145. A portion may comprise at least 25%, 50%, 60%, 70%, 80%, 90%, 95%, or 99% of a sequence set forth in any one of SEQ ID NOs: 1-13, 15, 17, 19-29, 31, 32, 34, 36, 38-44, 46, 48, 50-54, 56, 58-62, 64-66, and 68-145. In some embodiments, a nucleic acid sequence described by the disclosure is a nucleic acid sense strand (e.g., 5′ to 3′ strand), or in the context of a viral sequences a plus (+) strand. In some embodiments, a nucleic acid sequence described by the disclosure is a nucleic acid antisense strand (e.g., 3′ to 5′ strand), or in the context of viral sequences a minus (−) strand.

The skilled artisan recognizes that when referring to nucleic acid sequences comprising or encoding inhibitory nucleic acids (e.g., dsRNA, siRNA, miRNA, amiRNA, etc.) any one or more thymidine (T) nucleotides or uridine (U) nucleotides in a sequence provided herein may be replaced with any other nucleotide suitable for base pairing (e.g., via a Watson-Crick base pair) with an adenosine nucleotide. For example, T may be replaced with U, and U may be replaced with T.

An isolated nucleic acid as described herein may exist on its own, or as part of a vector. Generally, a vector can be a plasmid, cosmid, phagemid, bacterial artificial chromosome (BAC), or a viral vector (e.g., adenoviral vector, adeno-associated virus (AAV) vector, retroviral vector, baculoviral vector, etc.). In some embodiments, the vector is a plasmid (e.g., a plasmid comprising an isolated nucleic acid as described herein). In some embodiments, an rAAV vector is single-stranded (e.g., single-stranded DNA). In some embodiments, the vector is a recombinant AAV (rAAV) vector. In some embodiments, a vector is a Baculovirus vector (e.g., an Autographa californica nuclear polyhedrosis (AcNPV) vector).

Typically an rAAV vector (e.g., rAAV genome) comprises a transgene (e.g., an expression construct comprising one or more of each of the following: promoter, intron, enhancer sequence, protein coding sequence, inhibitory RNA coding sequence, polyA tail sequence, etc.) flanked by two AAV inverted terminal repeat (ITR) sequences. In some embodiments the transgene of an rAAV vector comprises an isolated nucleic acid as described by the disclosure. In some embodiments, each of the two ITR sequences of an rAAV vector is a full-length ITR (e.g., approximately 145 bp in length, and containing functional Rep binding site (RBS) and terminal resolution site (trs)). In some embodiments, one of the ITRs of an rAAV vector is truncated (e.g., shortened or not full-length). In some embodiments, a truncated ITR lacks a functional terminal resolution site (trs) and is used for production of self-complementary AAV vectors (scAAV vectors). In some embodiments, a truncated ITR is a ΔITR, for example as described by McCarty et al. (2003) Gene Ther. 10(26):2112-8.

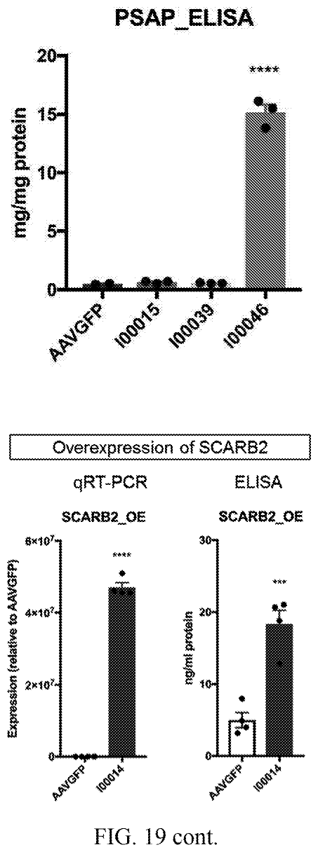

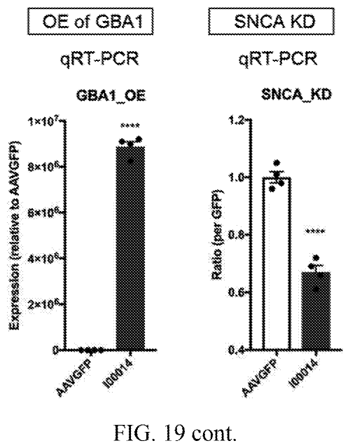

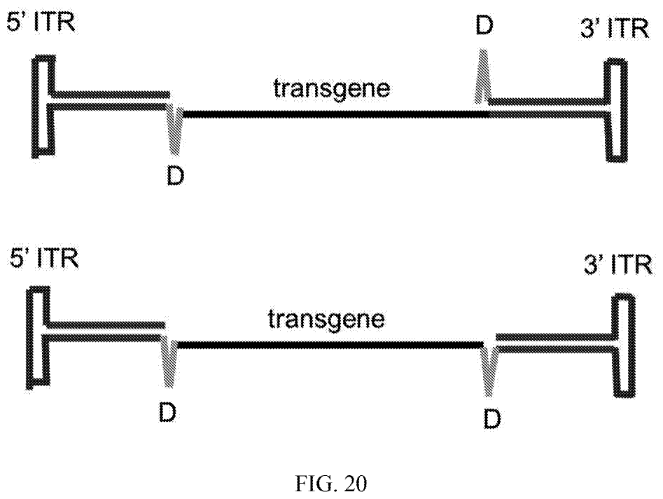

Aspects of the disclosure relate to isolated nucleic acids (e.g., rAAV vectors) comprising an ITR having one or more modifications (e.g., nucleic acid additions, deletions, substitutions, etc.) relative to a wild-type AAV ITR, for example relative to wild-type AAV2 ITR (e.g., SEQ ID NO: 29). The structure of wild-type AAV2 ITR is shown in FIG. 20. Generally, a wild-type ITR comprises a 125 nucleotide region that self-anneals to form a palindromic double-stranded T-shaped, hairpin structure consisting of two cross arms (formed by sequences referred to as B/B′ and C/C′, respectively), a longer stem region (formed by sequences A/A′), and a single-stranded terminal region referred to as the “D” region (FIG. 20). Generally, the “D” region of an ITR is positioned between the stem region formed by the A/A′ sequences and the insert containing the transgene of the rAAV vector (e.g., positioned on the “inside” of the ITR relative to the terminus of the ITR or proximal to the transgene insert or expression construct of the rAAV vector). In some embodiments, a “D” region comprises the sequence set forth in SEQ ID NO: 27. The “D” region has been observed to play an important role in encapsidation of rAAV vectors by capsid proteins, for example as disclosed by Ling et al. (2015) J Mol Genet Med 9(3).

The disclosure is based, in part, on that rAAV vectors comprising a “D” region located on the “outside” of the ITR (e.g., proximal to the terminus of the ITR relative to the transgene insert or expression construct) are efficiently encapsidated by AAV capsid proteins than rAAV vectors having ITRs with unmodified (e.g., wild-type) ITRs In some embodiments, rAAV vectors having a modified “D” sequence (e.g., a “D” sequence in the “outside” position) have reduced toxicity relative to rAAV vectors having wild-type ITR sequences.

In some embodiments, a modified “D” sequence comprises at least one nucleotide substitution relative to a wild-type “D” sequence (e.g., SEQ ID NO: 27). A modified “D” sequence may have at least 1, 2, 3, 4, 5, 6, 7, 8, 9, 10, or more than 10 nucleotide substitutions relative to a wild-type “D” sequence (e.g., SEQ ID NO: 27). In some embodiments, a modified “D” sequence comprises at least 10, 11, 12, 13, 14, 15, 16, 17, 18, or 19 nucleic acid substitutions relative to a wild-type “D” sequence (e.g., SEQ ID NO: 27). In some embodiments, a modified “D” sequence is between about 10% and about 99% (e.g., 10%, 15%, 20%, 25%, 30%, 40%, 45%, 50%, 55%, 60%, 65%, 70%, 75%, 80%, 85%, 90%, 95%, or 99%) identical to a wild-type “D” sequence (e.g., SEQ ID NO: 27). In some embodiments, a modified “D” sequence comprises the sequence set forth in SEQ ID NO: 26, also referred to as an “S” sequence as described in Wang et al. (1995) J Mol Biol 250(5):573-80.

An isolated nucleic acid or rAAV vector as described by the disclosure may further comprise a “TRY” sequence, for example as set forth in SEQ ID NO: 28 or as described by Francois, et al. 2005. The Cellular TATA Binding Protein Is Required for Rep-Dependent Replication of a Minimal Adeno-Associated Virus Type 2 p5 Element. J Virol. In some embodiments, a TRY sequence is positioned between an ITR (e.g. a 5′ ITR) and an expression construct (e.g. a transgene-encoding insert) of an isolated nucleic acid or rAAV vector.

Aspects of the disclosure relate to constructs which are configured to express one or more transgenes in myeloid cells (e.g., CNS myeloid cells, such as microglia) of a subject. Thus, in some embodiments, a construct (e.g., gene expression vector) comprises a protein coding sequence that is operably linked to a myeloid cell-specific promoter. Examples of myeloid cell-specific promoters include CD68 promoter, lysM promoter, csflr promoter, CD11c promoter, c-fes promoter, and F4/80 promoter, for example as described in Lin et al. Adv Exp Med Biol. 2010; 706:149-56. In some embodiments, a myeloid cell-specific promoter is a CD68 promoter or a F4/80 promoter.

In some aspects, the disclosure relates to Baculovirus vectors comprising an isolated nucleic acid or rAAV vector as described by the disclosure. In some embodiments, the Baculovirus vector is an Autographa californica nuclear polyhedrosis (AcNPV) vector, for example as described by Urabe et al. (2002) Hum Gene Ther 13(16):1935-43 and Smith et al. (2009) Mol Ther 17(11):1888-1896.

In some aspects, the disclosure provides a host cell comprising an isolated nucleic acid or vector as described herein. A host cell can be a prokaryotic cell or a eukaryotic cell. For example, a host cell can be a mammalian cell, bacterial cell, yeast cell, insect cell, etc. In some embodiments, a host cell is a mammalian cell, for example a HEK293T cell. In some embodiments, a host cell is a bacterial cell, for example an E. coli cell.

rAAVs

In some aspects, the disclosure relates to recombinant AAVs (rAAVs) comprising a transgene that encodes one or more isolated nucleic acids as described herein (e.g., an rAAV vector encoding 1, 2, 3, 4, 5, 6, 7, 8, 9, 10, or more gene products described herein and/or inhibitory nucleic acids targeting gene products described herein). The term “rAAVs” generally refers to viral particles comprising an rAAV vector encapsidated by one or more AAV capsid proteins. An rAAV described by the disclosure may comprise a capsid protein having a serotype selected from AAV1, AAV2, AAV3, AAV4, AAV5, AAV6, AAV7, AAV8, AAV9, and AAV10, or a variant thereof. In some embodiments, a capsid protein is an AAV9 capsid protein or a variant thereof. In some embodiments, an AAV9 capsid protein variant comprises a mutation at one or more positions corresponding to T492, Y705, and Y731 of SEQ ID NO: 147 (e.g., corresponding to those positions of AAV6). In some embodiments, the one or more mutations are selected from T492V, Y705F, Y731F, or a combination thereof. In some embodiments, an AAV9 capsid protein variant comprises the amino acid sequence set forth in SEQ ID NO: 149.

In some embodiments, an rAAV comprises a capsid protein from a non-human host, for example a rhesus AAV capsid protein such as AAVrh.10, AAVrh.39, etc. In some embodiments, an rAAV described by the disclosure comprises a capsid protein that is a variant of a wild-type capsid protein, such as a capsid protein variant that includes at least 1, 2, 3, 4, 5, 6, 7, 8, 9, 10, or more than 10 (e.g., 15, 20 25, 50, 100, etc.) amino acid substitutions (e.g., mutations) relative to the wild-type AAV capsid protein from which it is derived. In some embodiments, an AAV capsid protein variant is an AAV1RX capsid protein, for example as described by Albright et al. Mol Ther. 2018 Feb. 7; 26(2):510-523. In some embodiments, a capsid protein is AAV1RX and comprises the amino acid sequence set forth in SEQ ID NO: 146 (or is encoded by the nucleic acid sequence set forth in SEQ ID NO: 145). In some embodiments, a capsid protein variant is an AAV TM6 capsid protein, for example as described by Rosario et al. Mol Ther Methods Clin Dev. 2016; 3: 16026. In some embodiments, an AAV6 capsid protein variant is AAV-TM6 capsid protein and comprises the amino acid sequence set forth in SEQ ID NO: 148.

In some embodiments, rAAVs described by the disclosure readily spread through the CNS, particularly when introduced into the CSF space or directly into the brain parenchyma. Accordingly, in some embodiments, rAAVs described by the disclosure comprise a capsid protein that is capable of crossing the blood-brain barrier (BBB). For example, in some embodiments, an rAAV comprises a capsid protein having an AAV9 or AAVrh.10 serotype. Production of rAAVs is described, for example, by Samulski et al. (1989) J Virol. 63(9):3822-8 and Wright (2009) Hum Gene Ther. 20(7): 698-706. In some embodiments, an rAAV comprises a capsid protein that specifically or preferentially targets myeloid cells, for example microglial cells. In some embodiments, an rAAV transduces microglial cells.

In some embodiments, an rAAV as described by the disclosure (e.g., comprising a recombinant rAAV genome encapsidated by AAV capsid proteins to form an rAAV capsid particle) is produced in a Baculovirus vector expression system (BEVS). Production of rAAVs using BEVS are described, for example by Urabe et al. (2002) Hum Gene Ther 13(16):1935-43, Smith et al. (2009) Mol Ther 17(11):1888-1896, U.S. Pat. Nos. 8,945,918, 9,879,282, and International PCT Publication WO 2017/184879. However, an rAAV can be produced using any suitable method (e.g., using recombinant rep and cap genes). In some embodiments, an rAAV as disclosed herein is produced in HEK293 (human embryonic kidney) cells.

### Pharmaceutical Compositions

In some aspects, the disclosure provides pharmaceutical compositions comprising an isolated nucleic acid or rAAV as described herein and a pharmaceutically acceptable carrier. As used herein, the term “pharmaceutically acceptable” refers to a material, such as a carrier or diluent, which does not abrogate the biological activity or properties of the compound, and is relatively non-toxic, e.g., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

Compositions (e.g., pharmaceutical compositions) provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the compound or pharmaceutical composition described herein is suitable for topical administration to the eye of a subject.

In some embodiments, a composition comprises one or more (e.g., 1, 2, 3, 4, 5, 6, 7, 8, 9, or 10) different rAAVs, each rAAV comprising an isolated nucleic acid that encodes a different gene product (e.g., a different protein or inhibitory nucleic acid). The different rAAVs may comprise a capsid protein of the same serotype or different serotypes.

### Methods

Aspects of the disclosure relate to compositions for expression of one or more CNS disease-associated gene products in a subject to treat CNS-associated diseases. The one or more CNS disease-associated gene products may be encoded by one or more isolated nucleic acids or rAAV vectors. In some embodiments, a subject is administered a single vector (e.g., isolated nucleic acid, rAAV, etc.) encoding one or more (1, 2, 3, 4, 5, or more) gene products. In some embodiments, a subject is administered a plurality (e.g., 2, 3, 4, 5, or more) vectors (e.g., isolated nucleic acids, rAAVs, etc.), where each vector encodes a different CNS disease-associated gene product.

A CNS-associated disease may be a neurodegenerative disease, synucleinopathy, tauopathy, or a lysosomal storage disease. Examples of neurodegenerative diseases and their associated genes are listed in Table 2.

A “synucleinopathy” refers to a disease or disorder characterized by accumulation, overexpression or activity of alpha-Synuclein (the gene product of SNCA) in a subject (e.g., relative to a healthy subject, for example a subject not having a synucleinopathy). Examples of synucleinopathies and their associated genes are listed in Table 3.

A “tauopathy” refers to a disease or disorder characterized by accumulation, overexpression or activity of Tau protein in a subject (e.g., a healthy subject not having a tauopathy). Examples of tauopathies and their associated genes are listed in Table 4.

A “lysosomal storage disease” refers to a disease characterized by abnormal build-up of toxic cellular products in lysosomes of a subject. Examples of lysosomal storage diseases and their associated genes are listed in Table 5.

In some embodiments, the disclosure relates to methods of treating a disease selected from Parkinson's Disease (e.g., Parkinson's Disease with GBA1 mutation (PD-GBA), sporadic Parkinson's Disease (sPD)), Gaucher Disease (e.g., neuronopathic Gaucher disease (nGD), Type I Gaucher Disease (T1GD), Type II Gaucher Disease (T2GD), and Type III Gaucher Disease (T3GD)), Dementia with Lewy Bodies (DLB), Amyotrophic lateral sclerosis (ALS), and Niemann-Pick Type C disease (NPC) by administering to a subject in need thereof an isolated nucleic acid (e.g., an rAAV vector or rAAV comprising an isolated nucleic acid) that encodes GBA1.

In some embodiments, the disclosure relates to methods of treating Frontotemporal Dementia (e.g., Frontotemporal Dementia with GRN mutation (FTD-GRN), Frontotemporal Dementia with MAPT mutation (FTD-tau), and Frontotemporal Dementia with C9ORF72 mutation (FTD-C9orf72)), Parkinson's Disease (PD), Alzheimer's Disease (AD), Neuronal Ceroid Lipofuscinosis (NCL), Corticobasal Degeneration (CBD), Motor Neuron Disease (MND), or Gaucher Disease (GD) by administering to a subject in need thereof an isolated nucleic acid (e.g., an rAAV vector or rAAV comprising an isolated nucleic acid) that encodes PGRN (also referred to as GRN).

In some embodiments, the disclosure relates to methods of treating Synucleinopathies (e.g., multiple system atrophy (MSA), Parkinson's Disease (PD), Parkinson's disease with GBA1 mutation (PD-GBA), Dementia with Lewy Bodies (DLB), Dementia with Lewy Bodies with GBA1 mutation, and Lewy Body Disease) by administering to a subject in need thereof an isolated nucleic acid (e.g., an rAAV vector or rAAV comprising an isolated nucleic acid) that encodes GBA1 gene product, and an inhibitory nucleic acid targeting SNCA.

In some embodiments, the disclosure relates to methods of treating a disease selected from Parkinson's Disease (PD), Frontotemporal Dementia (e.g., Frontotemporal Dementia with GRN mutation (FTD-GRN)), Lysosomal Storage Diseases (LSDs), or Gaucher Disease (GD) by administering to a subject in need thereof an isolated nucleic acid (e.g., an rAAV vector or rAAV comprising an isolated nucleic acid) that encodes PSAP.

In some embodiments, the disclosure relates to methods of treating Alzheimer's Disease (AD), Nasu-Hakola Disease (NHD) Frontotemporal Dementia with MAPT mutation (FTD-Tau), or Parkinson's Disease (PD), by administering to a subject in need thereof an isolated nucleic acid (e.g., an rAAV vector or rAAV comprising an isolated nucleic acid) that encodes TREM2.

In some embodiments, the disclosure relates to methods of treating Alzheimer's disease (AD) or Frontotemporal Dementia (Frontotemporal Dementia with MAPT mutation (FTD-Tau), a tauopathy, Progressive supranuclear palsy (PSP), neurodegenerative disease, Lewy Body Disease (LBD) or Parkinson's Disease by administering to a subject in need thereof an isolated nucleic acid (e.g., an rAAV vector or rAAV comprising an isolated nucleic acid) that encodes inhibitory nucleic acids targeting MAPT.

As used herein “treat” or “treating” refers to (a) preventing or delaying onset of a CNS disease; (b) reducing severity of a CNS disease; (c) reducing or preventing development of symptoms characteristic of a CNS disease; (d) and/or preventing worsening of symptoms characteristic of a CNS disease. Symptoms of CNS disease may include, for example, motor dysfunction (e.g., shaking, rigidity, slowness of movement, difficulty with walking, paralysis), cognitive dysfunction (e.g., dementia, depression, anxiety, psychosis), difficulty with memory, emotional and behavioral dysfunction.

The disclosure is based, in part, on compositions for expression of combinations of CNS diseases-associated genes (e.g., PD-associated gene products) in a subject that act together (e.g., synergistically) to treat the disease.

Accordingly, in some aspects, the disclosure provides a method for treating a subject having or suspected of having CNS-associated diseases (e.g., Parkinson's disease, AD, FTD, etc.), the method comprising administering to the subject a composition (e.g., a composition comprising an isolated nucleic acid or a vector or a rAAV) as described by the disclosure.

In some embodiments, a subject has one or more signs or symptoms, or has a genetic predisposition (e.g., a mutation in a gene listed in Table 1) to a neurodegenerative disease listed in Table 2. In some embodiments, a subject has one or more signs or symptoms, or has a genetic predisposition (e.g., a mutation in a gene listed in Table 1) to a synucleinopathy listed in Table 3. In some embodiments, a subject has one or more signs or symptoms, or has a genetic predisposition (e.g., a mutation in a gene listed in Table 1) to a tauopathy listed in Table 4. In some embodiments, a subject has one or more signs or symptoms, or has a genetic predisposition (e.g., a mutation in a gene listed in Table 1) to a lysosomal storage disease listed in Table 5.

The disclosure is based, in part, on compositions for expression of one or more CNS-disease associated gene products in a subject to treat Gaucher disease. In some embodiments, the Gaucher disease is a neuronopathic Gaucher disease, for example Type 2 Gaucher disease or Type 3 Gaucher disease. In some embodiments, a subject does not have PD or PD symptoms.

Accordingly, in some aspects, the disclosure provides a method for treating a subject having or suspected of having neuronopathic Gaucher disease, the method comprising administering to the subject a composition (e.g., a composition comprising an isolated nucleic acid or a vector or a rAAV) as described by the disclosure.

The disclosure is based, in part, on compositions for expression of one or more CNS-disease associated gene products in a subject to treat Alzheimer's disease or fronto-temporal dementia (FTD). In some embodiments, the subject does not have Alzheimer's disease.

Accordingly, in some aspects, the disclosure provides a method for treating a subject having or suspected of having FTD, the method comprising administering to the subject a composition (e.g., a composition comprising an isolated nucleic acid or a vector or a rAAV) as described by the disclosure. In some embodiments, a subject having Alzheimer's disease or fronto-temporal dementia (FTD) is administered an rAAV encoding Progranulin (PGRN, also referred to as GRN) or a portion thereof.

In some aspects, the disclosure provides a method for delivering a transgene to microglial cells, the method comprising administering an rAAV as described herein to a subject.

In some embodiments, a rAAV encoding a Gcase protein for treating Type 2 or Type 3 Gaucher disease or Parkinson's disease with a GBA1 mutation is administered to a subject as a single dose, and the rAAV is not administered to the subject subsequently.

In some embodiments, a rAAV encoding a Gcase protein is administered via a single suboccipital injection into the cisterna magna. In some embodiments, the injection into the cisterna magna is performed under radiographic guidance.

A subject is typically a mammal, preferably a human. In some embodiments, a subject is between the ages of 1 month old and 10 years old (e.g., 1 month, 2 months, 3 months, 4, months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 3, years, 4 years, 5 years, 6 years, 7 years, 8 years, 9 years, 10 years, or any age therebetween). In some embodiments, a subject is between 2 years old and 20 years old. In some embodiments, a subject is between 30 years old and 100 years old. In some embodiments, a subject is older than 55 years old.

In some embodiments, a composition is administered directly to the CNS of the subject, for example by direct injection into the brain and/or spinal cord of the subject. Examples of CNS-direct administration modalities include but are not limited to intracerebral injection, intraventricular injection, intracisternal injection, intraparenchymal injection, intrathecal injection, and any combination of the foregoing. In some embodiments, a composition is administered to a subject by intra-cisterna magna (ICM) injection. In some embodiments, direct injection into the CNS of a subject results in transgene expression (e.g., expression of the first gene product, second gene product, and if applicable, third gene product) in the midbrain, striatum and/or cerebral cortex of the subject. In some embodiments, direct injection into the CNS results in transgene expression (e.g., expression of the first gene product, second gene product, and if applicable, third gene product) in the spinal cord and/or CSF of the subject.

In some embodiments, direct injection to the CNS of a subject comprises convection enhanced delivery (CED). Convection enhanced delivery is a therapeutic strategy that involves surgical exposure of the brain and placement of a small-diameter catheter directly into a target area of the brain, followed by infusion of a therapeutic agent (e.g., a composition or rAAV as described herein) directly to the brain of the subject. CED is described, for example by Debinski et al. (2009) Expert Rev Neurother. 9(10):1519-27.

In some embodiments, a composition is administered peripherally to a subject, for example by peripheral injection. Examples of peripheral injection include subcutaneous injection, intravenous injection, intra-arterial injection, intraperitoneal injection, or any combination of the foregoing. In some embodiments, the peripheral injection is intra-arterial injection, for example injection into the carotid artery of a subject.

In some embodiments, a composition (e.g., a composition comprising an isolated nucleic acid or a vector or a rAAV) as described by the disclosure is administered both peripherally and directly to the CNS of a subject. For example, in some embodiments, a subject is administered a composition by intra-arterial injection (e.g., injection into the carotid artery) and by intraparenchymal injection (e.g., intraparenchymal injection by CED). In some embodiments, the direct injection to the CNS and the peripheral injection are simultaneous (e.g., happen at the same time). In some embodiments, the direct injection occurs prior (e.g., between 1 minute and 1 week, or more before) to the peripheral injection. In some embodiments, the direct injection occurs after (e.g., between 1 minute and 1 week, or more after) the peripheral injection.

In some embodiments, a subject is administered an immunosuppressant prior to (e.g., between 1 month and 1 minute prior to) or at the same time as a composition as described herein. In some embodiments, the immunosuppressant is a corticosteroid (e.g., prednisone, budesonide, etc.), an mTOR inhibitor (e.g., sirolimus, everolimus, etc.), an antibody (e.g., adalimumab, etanercept, natalizumab, etc.), or methotrexate.

The amount of composition (e.g., a composition comprising an isolated nucleic acid or a vector or a rAAV) as described by the disclosure administered to a subject will vary depending on the administration method. For example, in some embodiments, a rAAV as described herein is administered to a subject at a titer between about 10⁹ Genome copies (GC)/kg and about 10¹⁴ GC/kg (e.g., about 10⁹ GC/kg, about 10¹⁰ GC/kg, about 10¹¹ GC/kg, about 10¹² GC/kg, about 10¹² GC/kg, or about 10¹⁴ GC/kg). In some embodiments, a subject is administered a high titer (e.g., >10¹² Genome Copies GC/kg of an rAAV) by injection to the CSF space, or by intraparenchymal injection. In some embodiments, a rAAV as described herein is administered to a subject at a dose ranging from about 1×10¹⁰ vector genomes (vg) to about 1×10¹⁷ vg by intravenous injection. In some embodiments, a rAAV as described herein is administered to a subject at a dose ranging from about 1×10¹⁰ vg to about 1×10¹⁶ vg by injection into the cisterna magna.

A composition (e.g., a composition comprising an isolated nucleic acid or a vector or a rAAV) as described by the disclosure can be administered to a subject once or multiple times (e.g., 2, 3, 4, 5, 6, 7, 8, 9, 10, 20, or more) times. In some embodiments, a composition is administered to a subject continuously (e.g., chronically), for example via an infusion pump.

TABLE 2
Examples of neurodegenerative diseases

| Disease | Associated genes |
|---|---|
| Alzheimer's disease | APP, PSEN1, PSEN2, APOE |
| Parkinson's disease | LRRK2, PARK7, PINK1, PRKN, SNCA, GBA, |
| | UCHL1, ATP13A2, VPS35 |
| Huntington's disease | HTT |
| Amyotrophic lateral sclerosis | ALS2, ANG, ATXN2, C9orf72, CHCHD10, |
| | CHMP2B, DCTN1, ERBB4, FIG4, FUS, |
| | HNRNPA1, MATR3, NEFH, OPTN, PFN1, |
| | PRPH, SETX, SIGMAR1, SMN1, SOD1, |
| | SPG11, SQSTM1, TARDBP, TBK1, TRPM7, |
| | TUBA4A, UBQLN2, VAPB, VCP |
| Batten disease (Neuronal ceroid | PPT1, TPP1, CLN3, CLN5, CLN6, MFSD8, |
| lipofunscinosis) | CLN8, CTSD, DNAJC5, CTSF, ATP13A2, |
| | GRN, KCTD7 |
| Friedreich's ataxia | FXN |
| Lewy body disease | APOE, GBA, SNCA, SNCB |
| Spinal muscular atrophy | SMN1, SMN2 |
| Multiple sclerosis | CYP27B1, HLA-DRB1, IL2RA, IL7R, |
| | TNFRSF1A |
| Prion disease (Creutzfeldt-Jakob disease, Fatal | PRNP |
| familial insomnia, Gertsmann-Straussler- | |
| Scheinker syndrome, Variably protease- | |
| sensitive prionopathy) | |

TABLE 3
Examples of synucleinopathies

| Disease | Associated genes |
|---|---|
| Parkinson's disease | LRRK2, PARK7, PINK1, PRKN, SNCA, GBA, |
| | UCHL1, ATP13A2, VPS35 |
| Dementia with Lewy bodies | APOE, GBA, SNCA, SNCB |
| Multiple system atrophy | COQ2, SNCA |

TABLE 4
Examples of tauopathies

| Disease | Associated genes |
|---|---|
| Alzheimer's disease | APP, PSEN1, PSEN2, APOE |
| Primary age-related tauopathy | MAPT |
| Progressive supranuclear palsy | MAPT |
| Corticobasal degeneration | MAPT, GRN, C9orf72, VCP, |
| | CHMP2B, TARDBP, FUS |
| Frontotemporal dementia with | MAPT |
| parkinsonism-17 | |
| Subacute sclerosing panencephalitis | SCN1A |
| Lytico-Bodig disease | |
| Gangioglioma, gangliocytoma | |
| Meningioangiomatosis | |
| Postencephalitic parkinsonism | |
| Chronic traumatic encephalopathy | |

TABLE 5
Examples of lysosomal storage diseases

| Disease | Associated genes |
|---|---|
| Niemann-Pick disease | NPC1, NPC2, SMPD1 |
| Fabry disease | GLA |
| Krabbe disease | GALC |
| Gaucher disease | GBA |
| Tach-Sachs disease | HEXA |
| Metachromatic leukodystrophy | ARSA, PSAP |
| Farber disease | ASAH1 |
| Galactosialidosis | CTSA |
| Schindler disease | NAGA |
| GM1 gangliosidosis | GLB1 |
| GM2 gangliosidosis | GM2A |
| Sandhoff disease | HEXB |
| Lysosomal acid lipase deficiency | LIPA |
| Multiple sulfatase deficiency | SUMF1 |
| Mucopolysaccharidosis Type I | IDUA |
| Mucopolysaccharidosis Type II | IDS |
| Mucopolysaccharidosis Type III | GNS, HGSNAT, NAGLU, SGSH |
| Mucopolysaccharidosis Type IV | GALNS, GLB1 |
| Mucopolysaccharidosis Type VI | ARSB |
| Mucopolysaccharidosis Type VII | GUSB |
| Mucopolysaccharidosis Type IX | HYAL1 |
| Mucolipidosis Type II | GNPTAB |
| Mucolipidosis Type III alpha/beta | GNPTAB |
| Mucolipidosis Type III gamma | GNPTG |
| Mucolipidosis Type IV | MCOLN1 |
| Neuronal ceroid lipofuscinosis | PPT1, TPP1, CLN3, CLN5, CLN6, MFSD8, |
| | CLN8, CTSD, DNAJC5, CTSF, ATP13A2, |
| | GRN, KCTD7 |
| Alpha-mannosidosis | MAN2B1 |
| Beta-mannosidosis | MANBA |
| Aspartylglucosaminuria | AGA |
| Fucosidosis | FUCA1 |

### EXAMPLES

### Example 1: rAAV Vectors

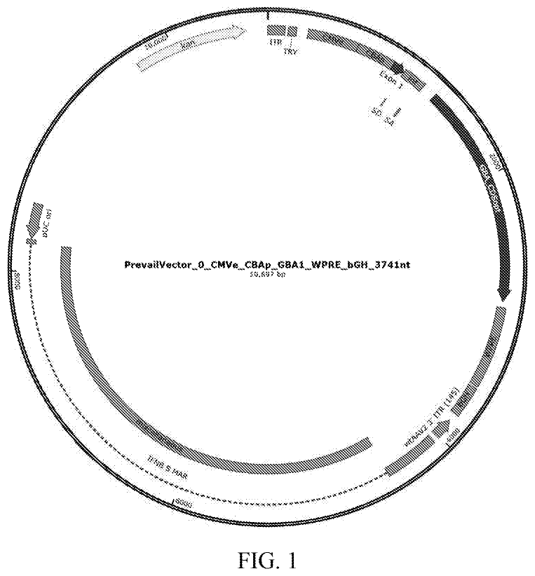

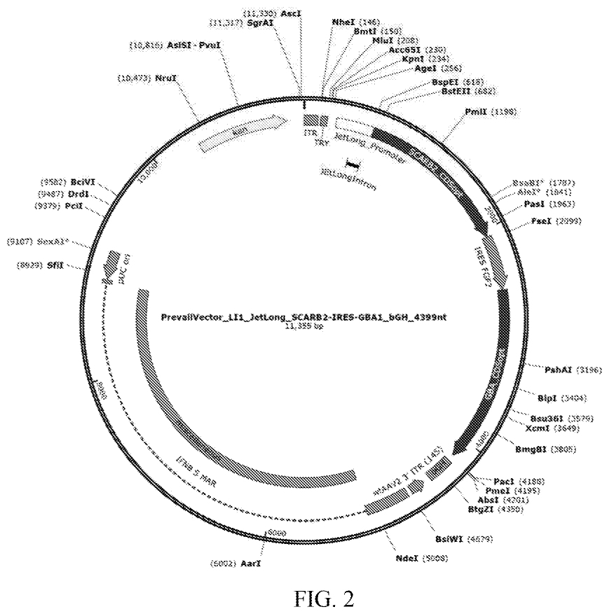

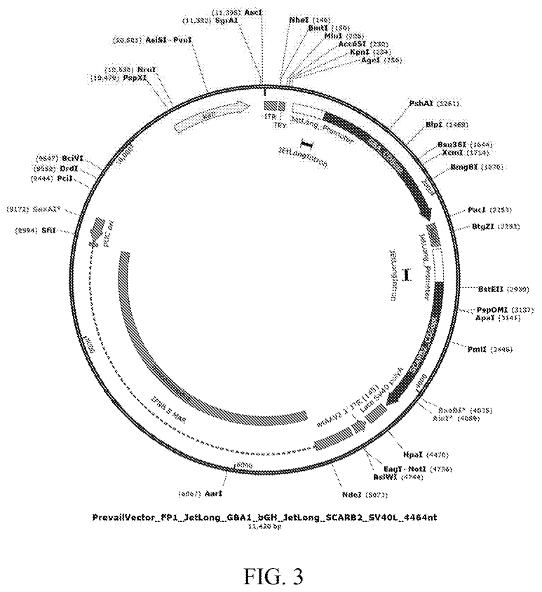

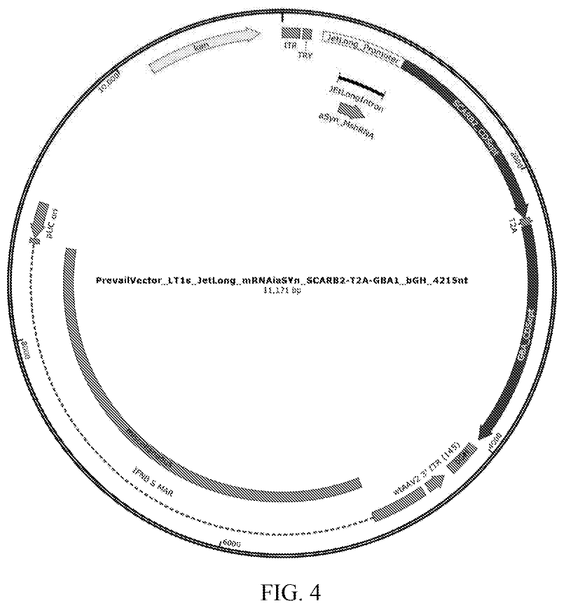

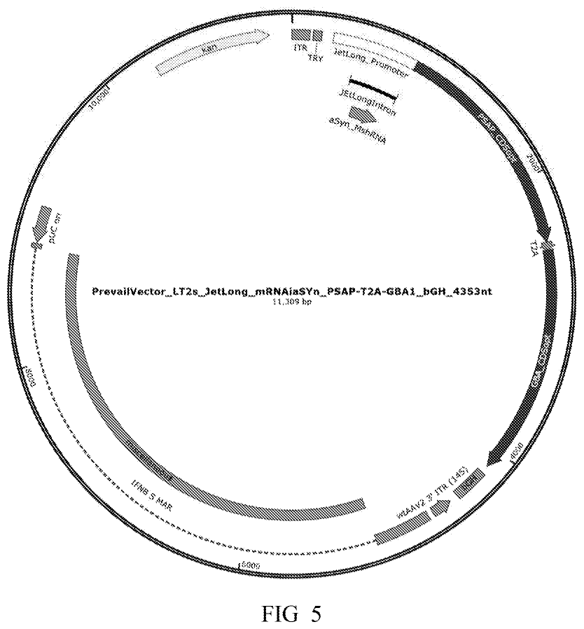

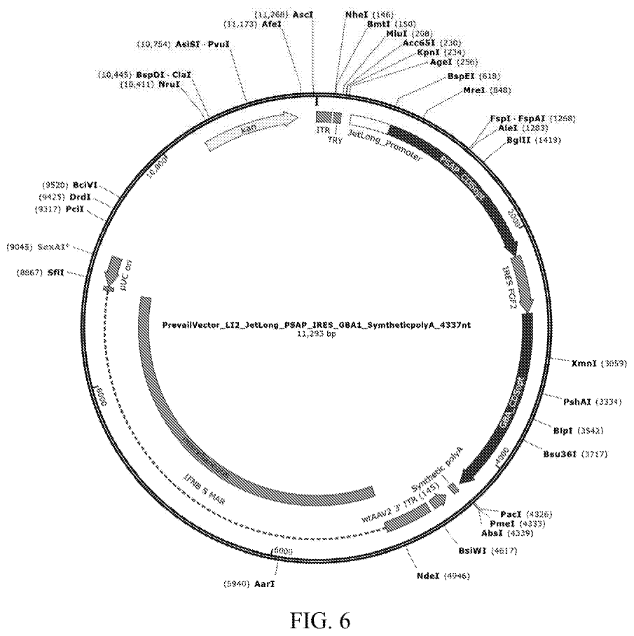

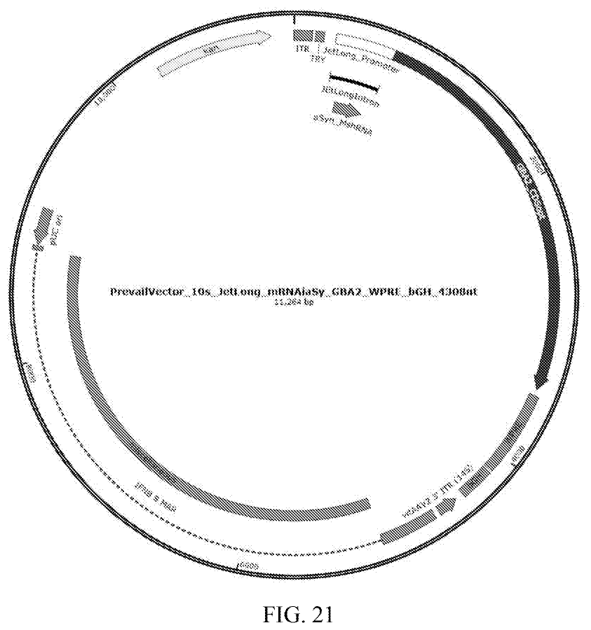

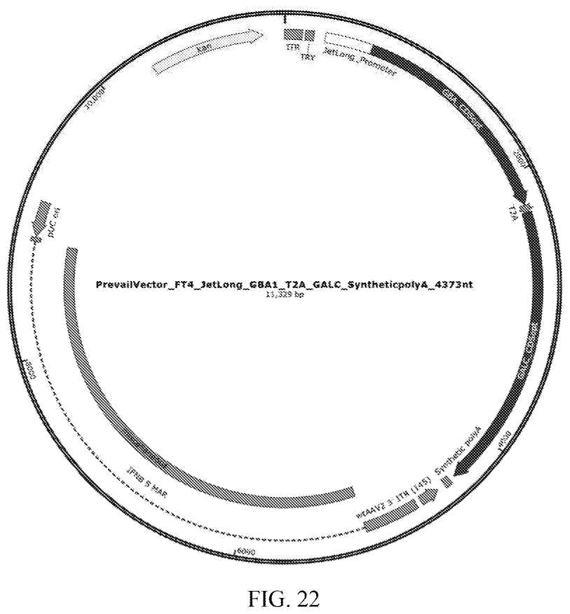

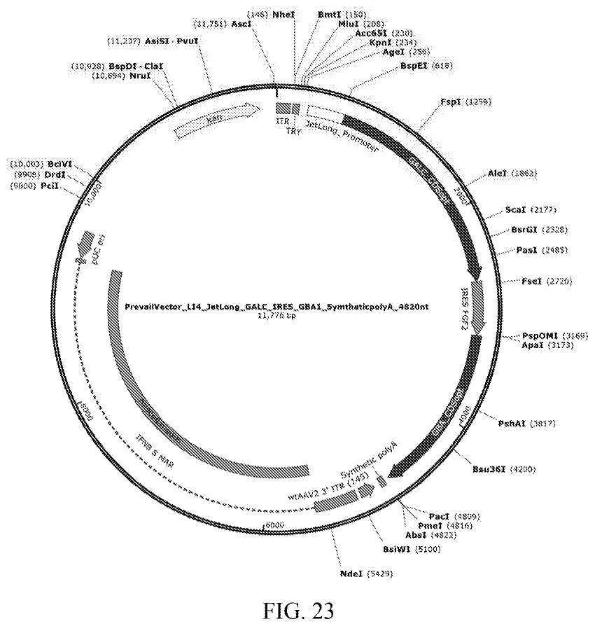

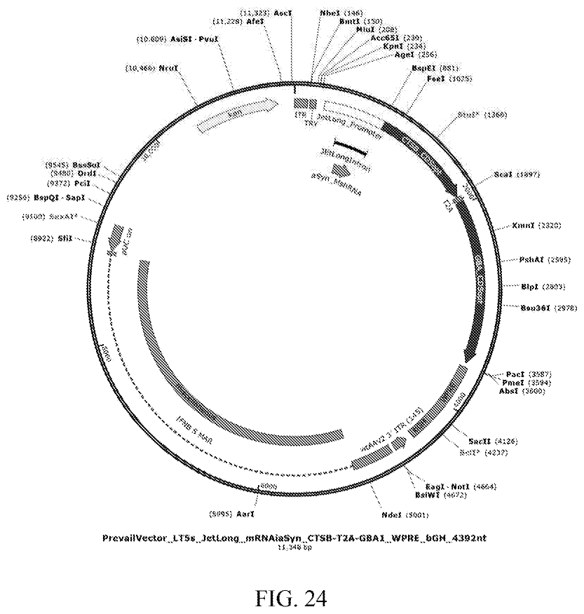

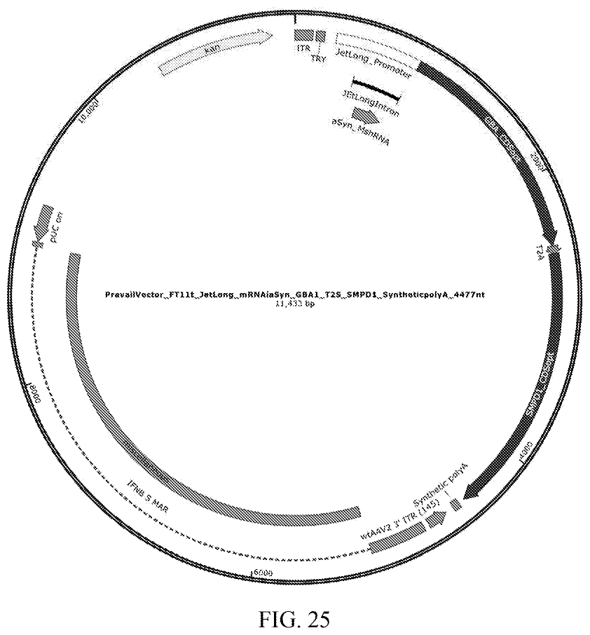

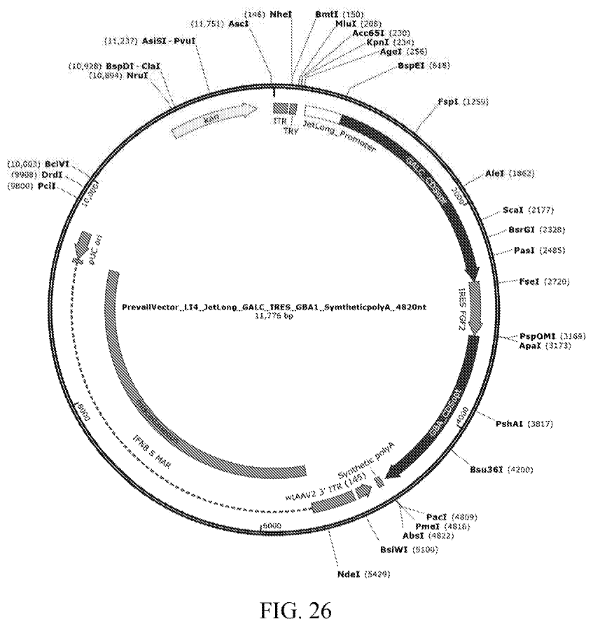

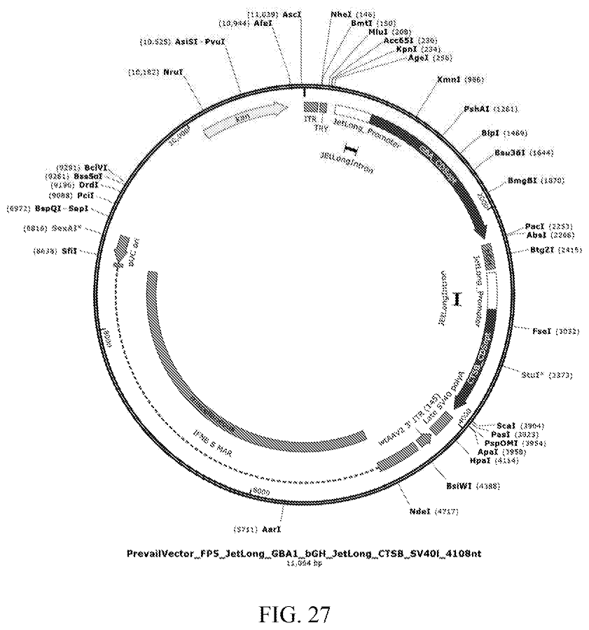

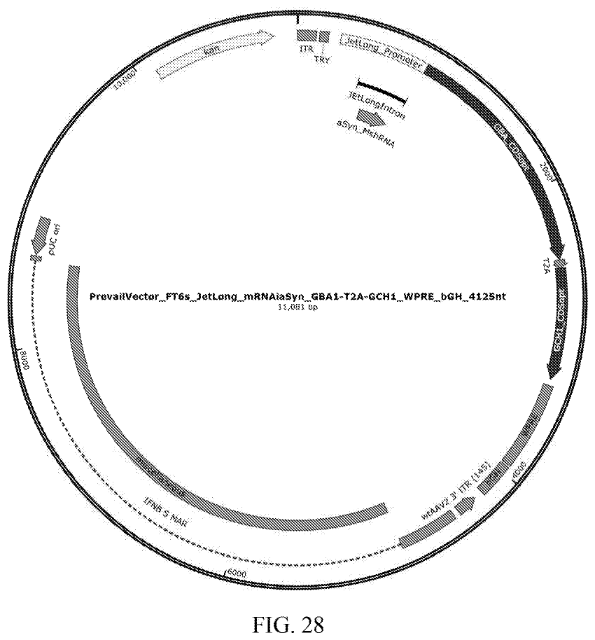

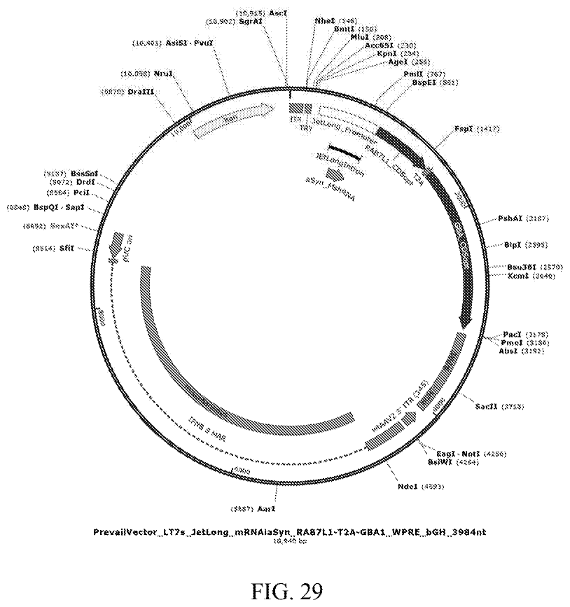

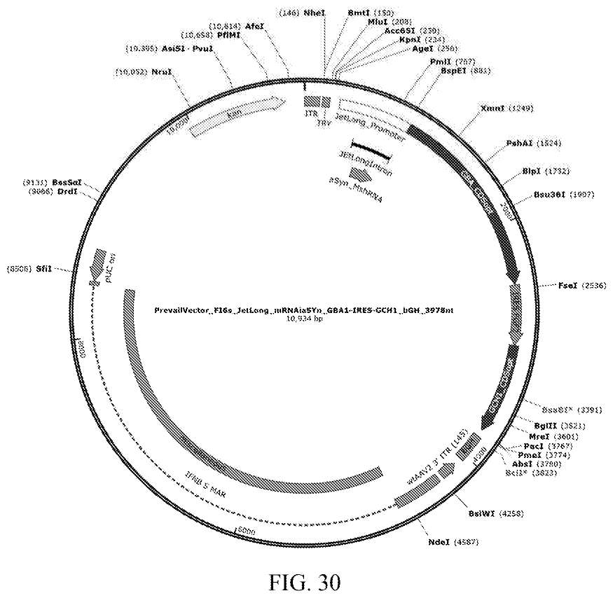

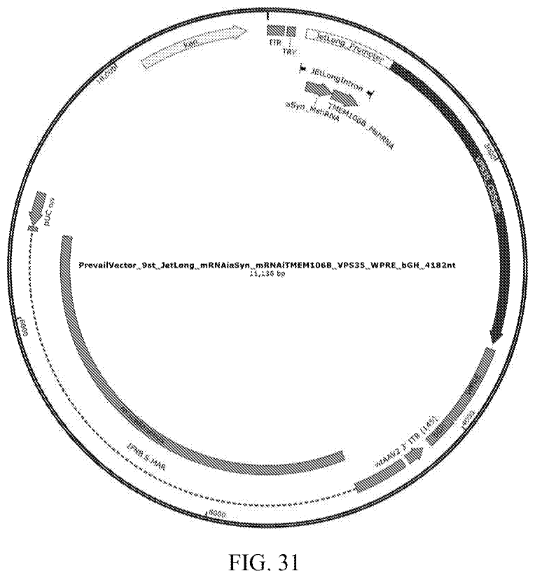

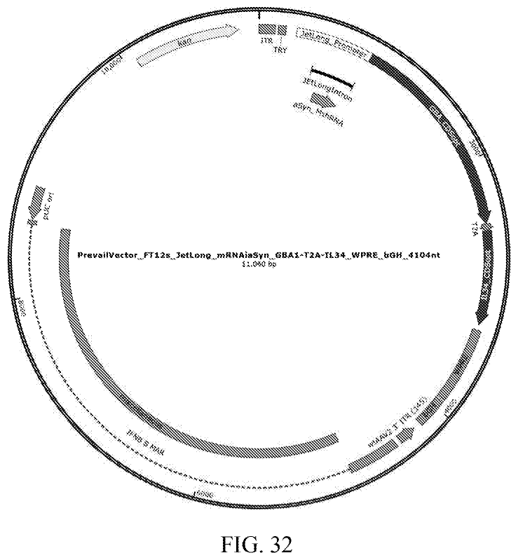

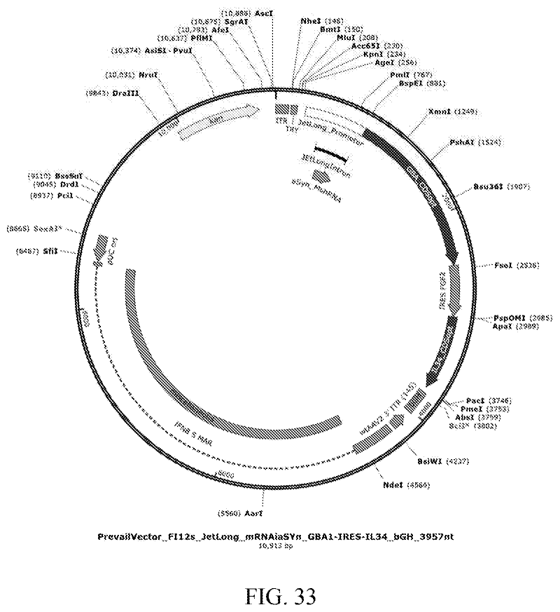

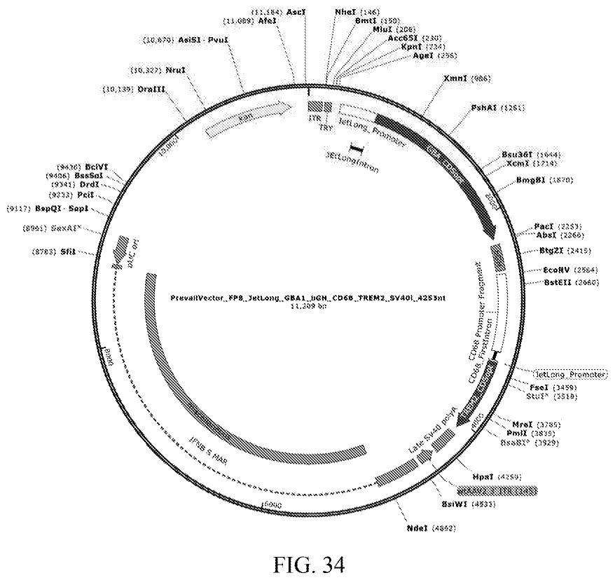

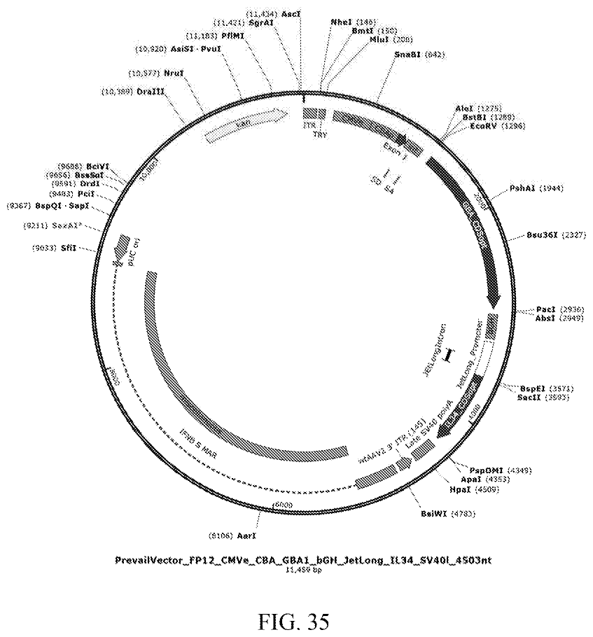

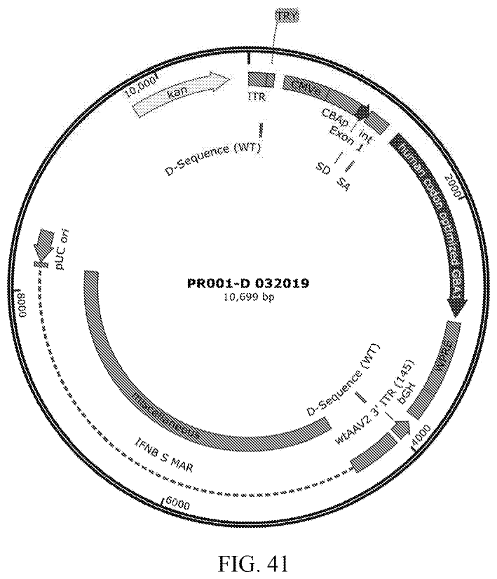

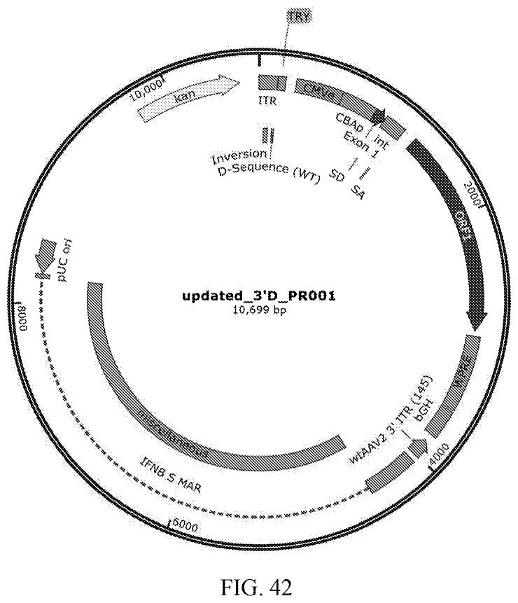

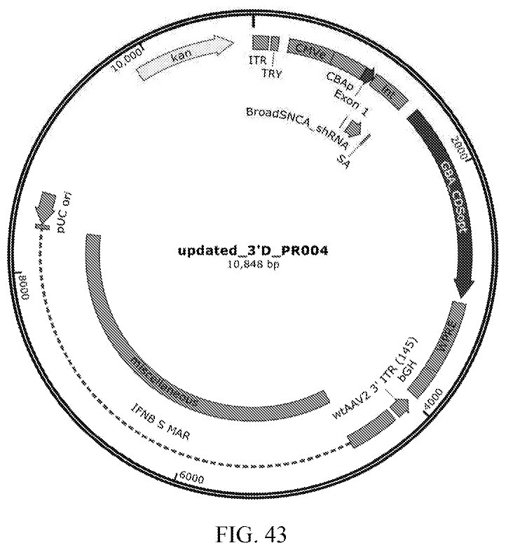

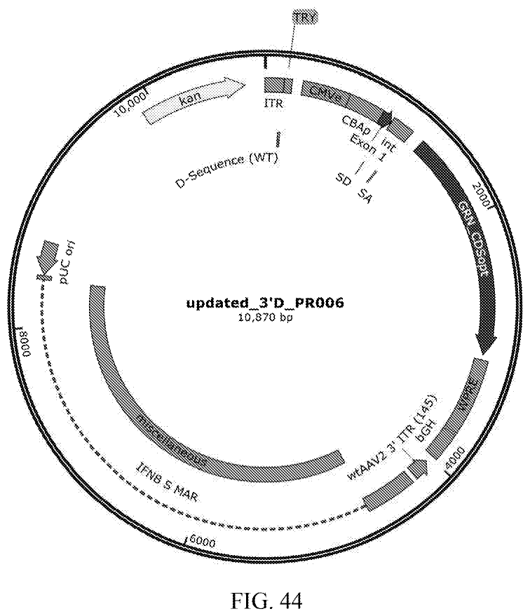

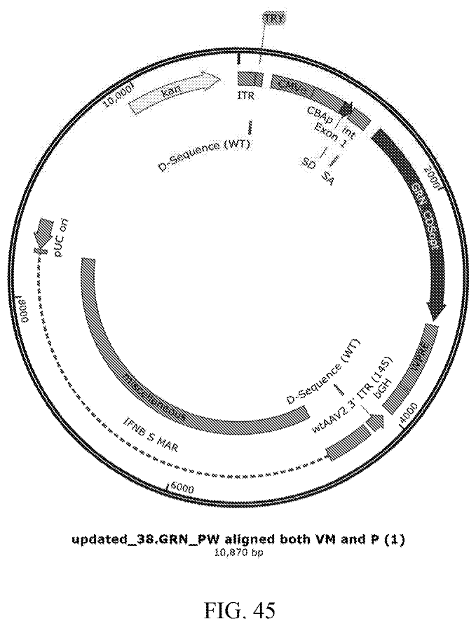

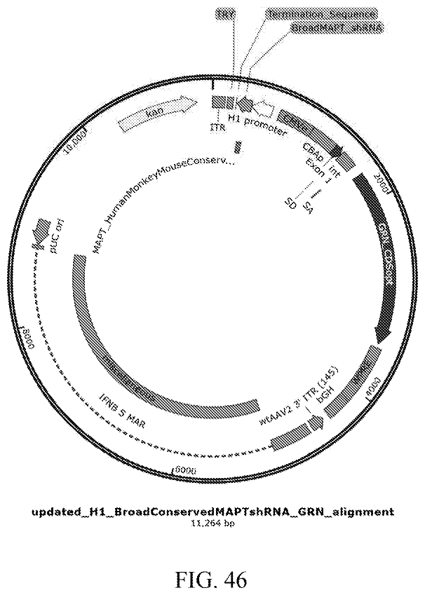

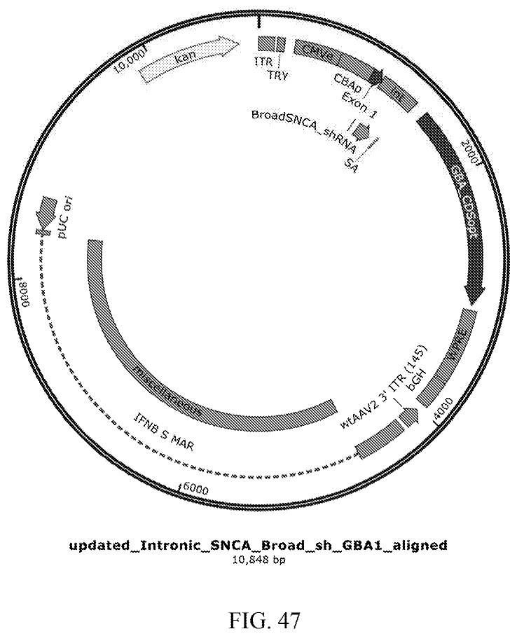

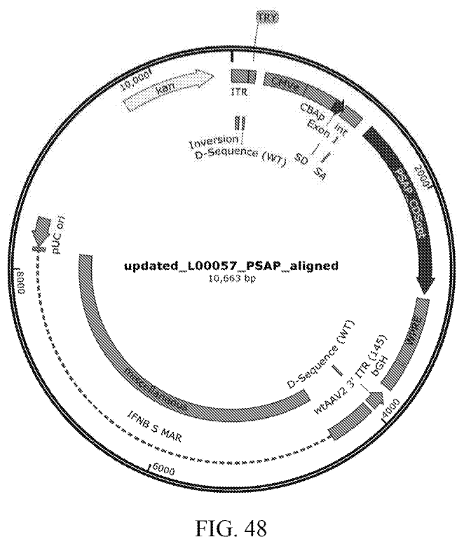

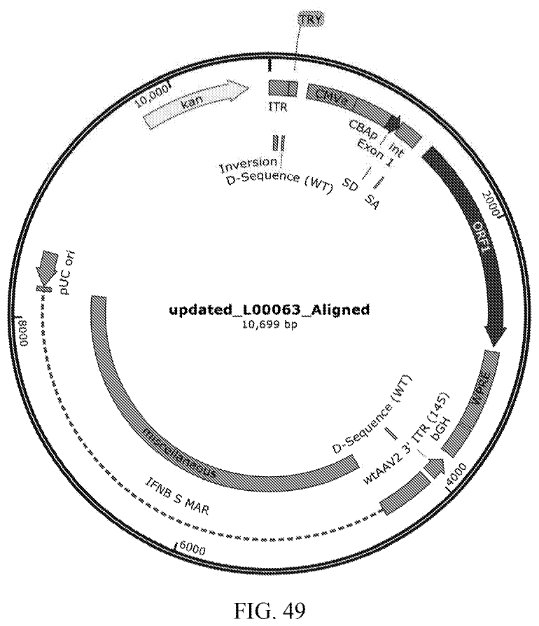

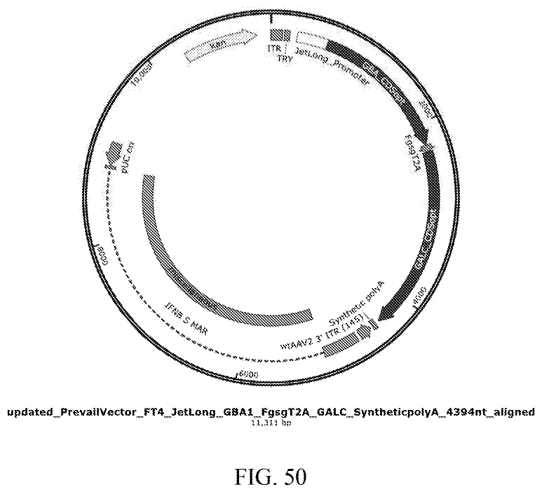

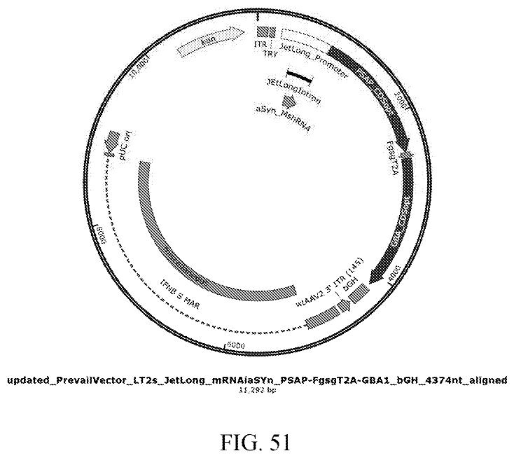

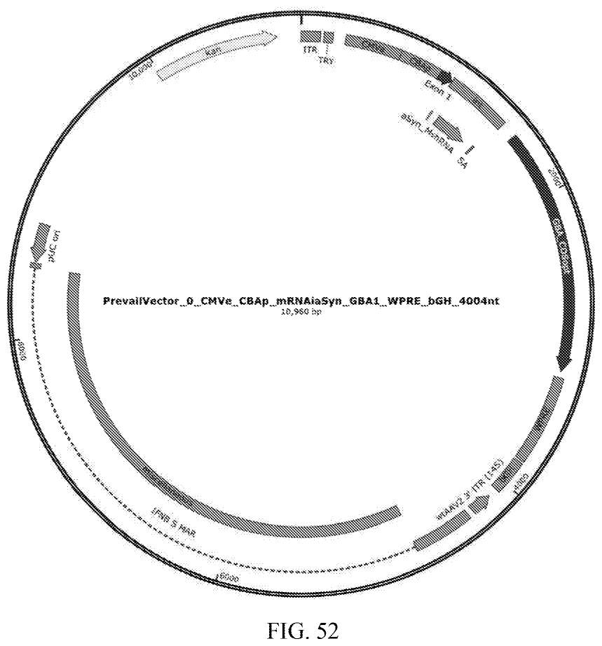

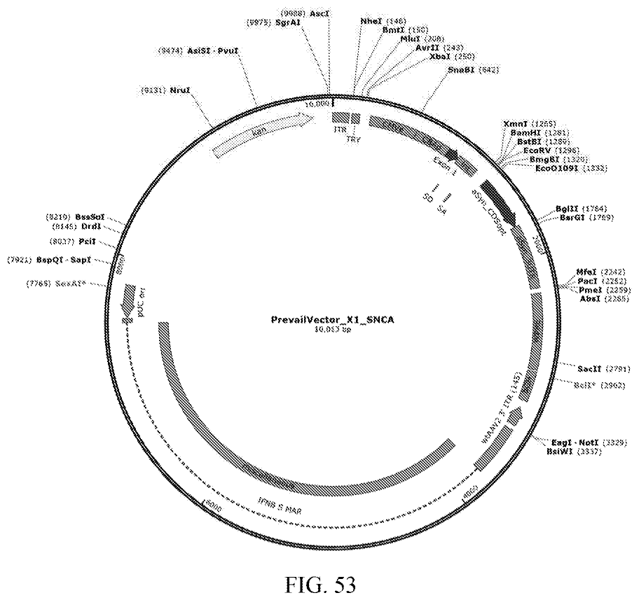

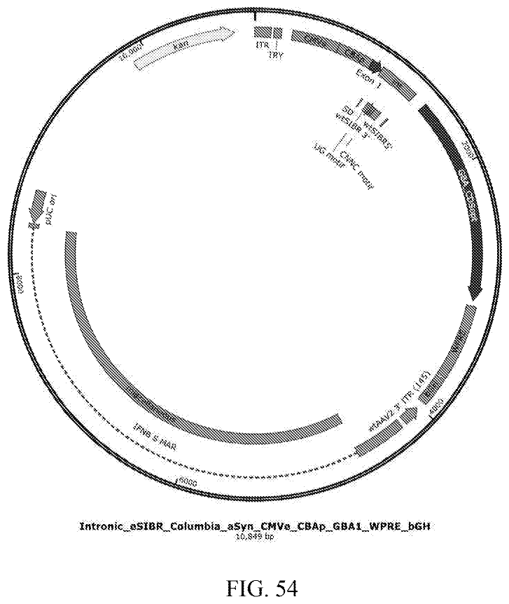

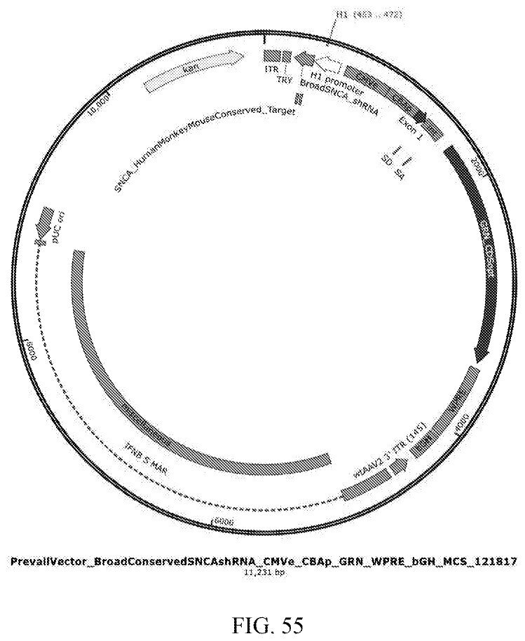

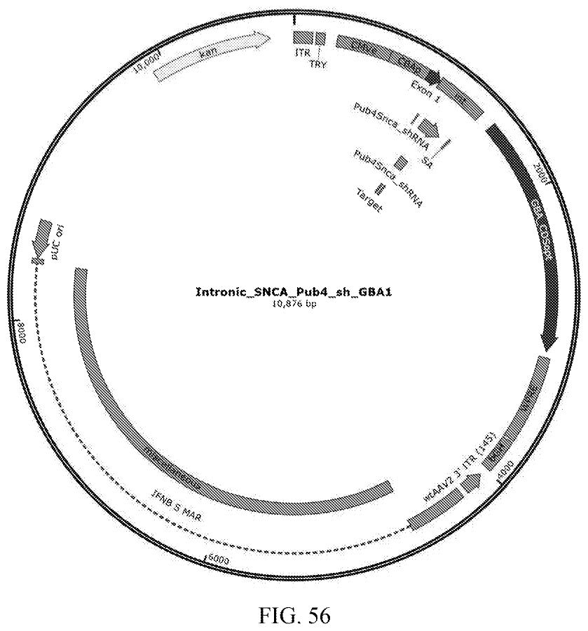

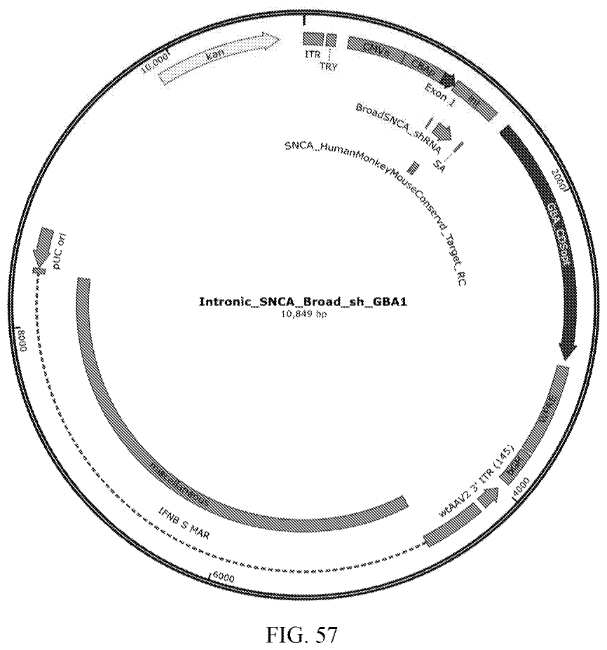

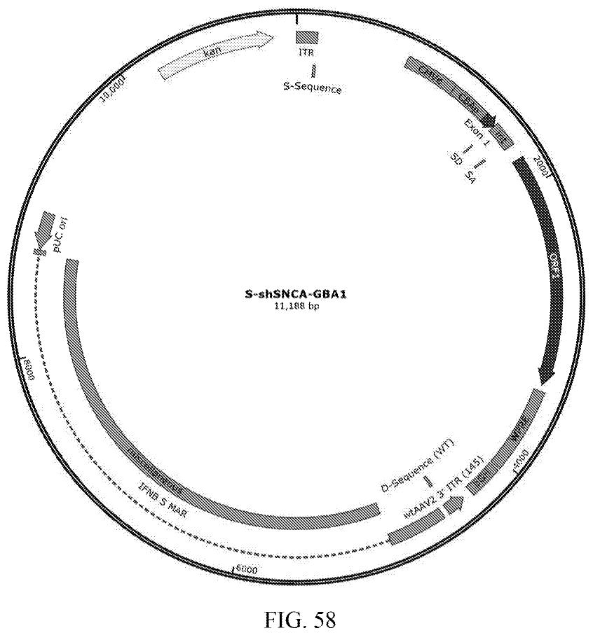

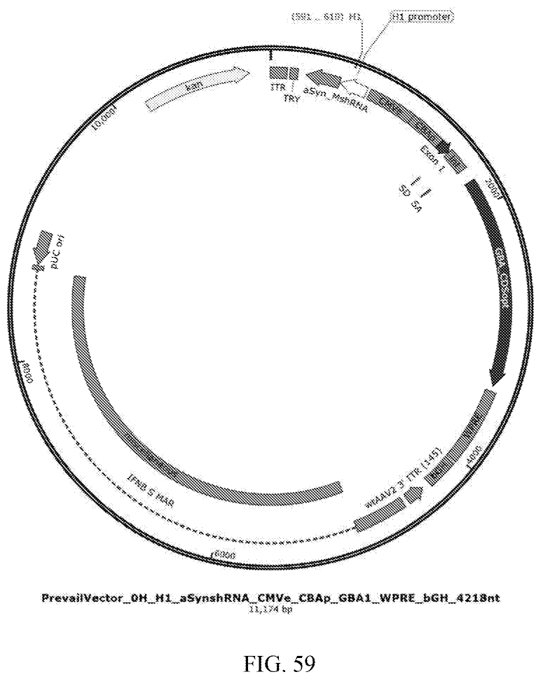

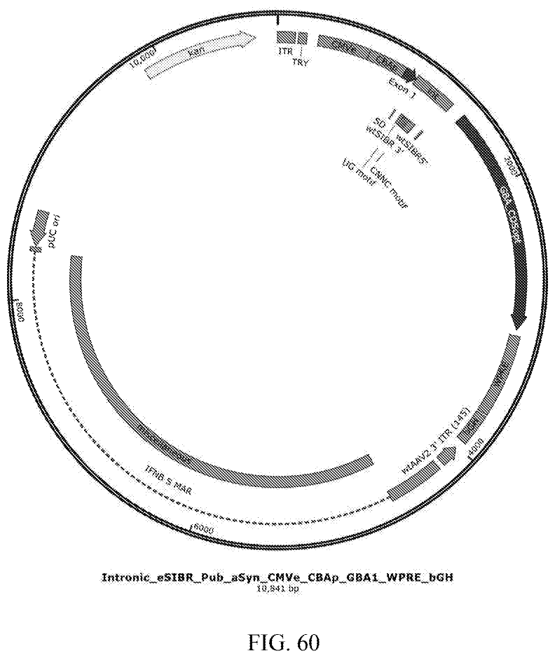

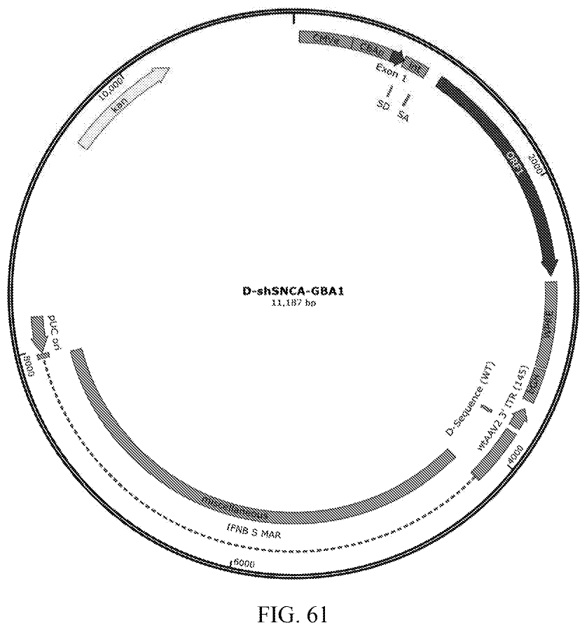

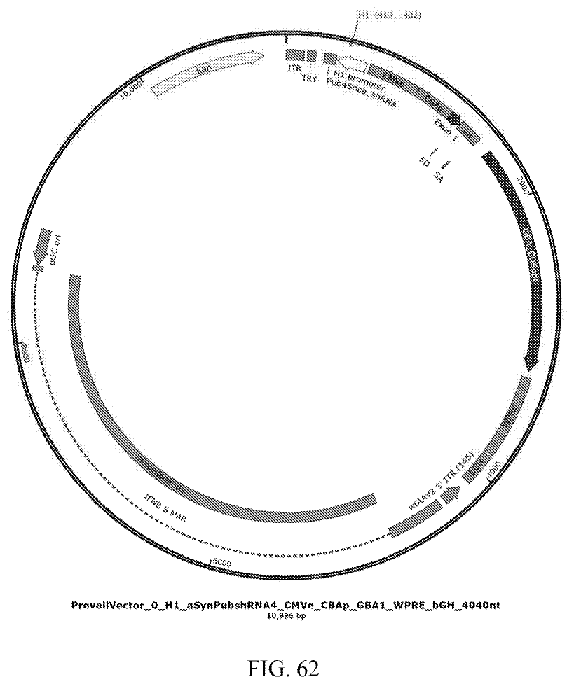

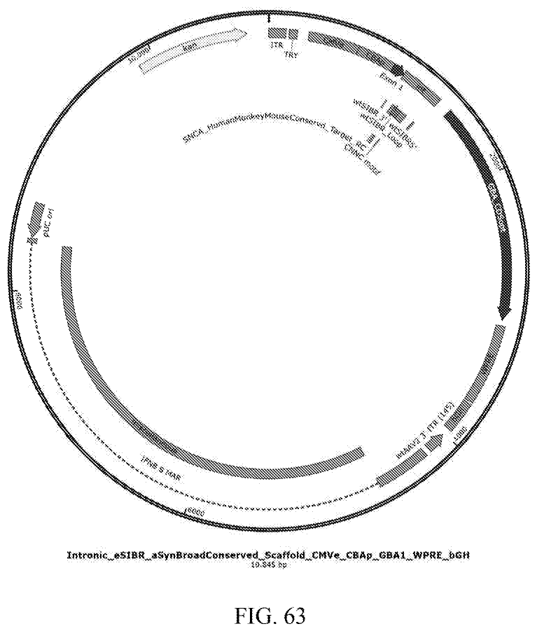

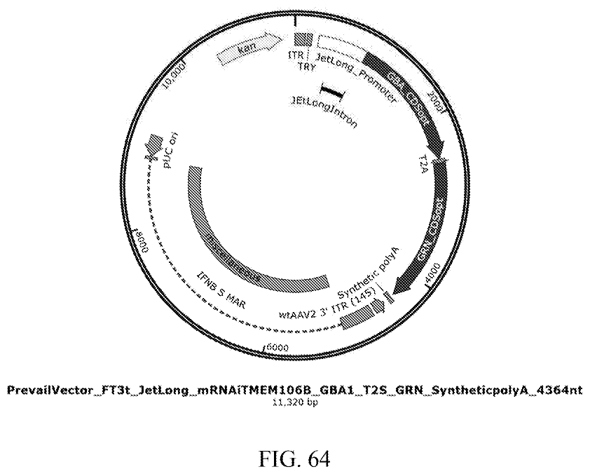

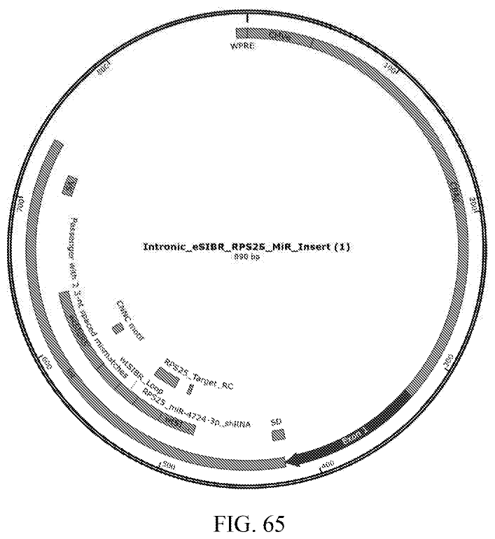

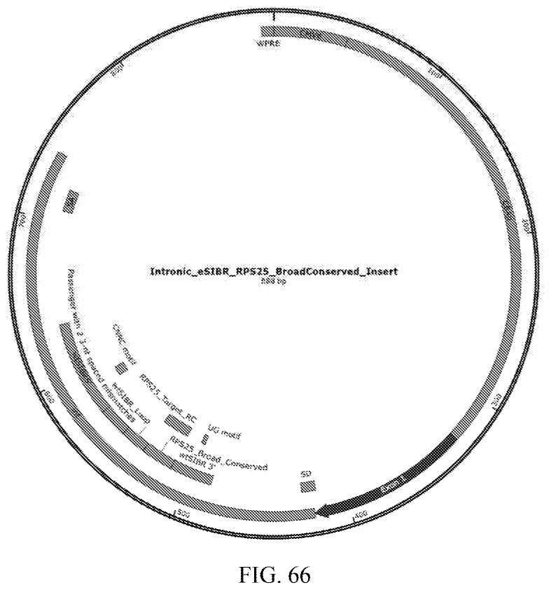

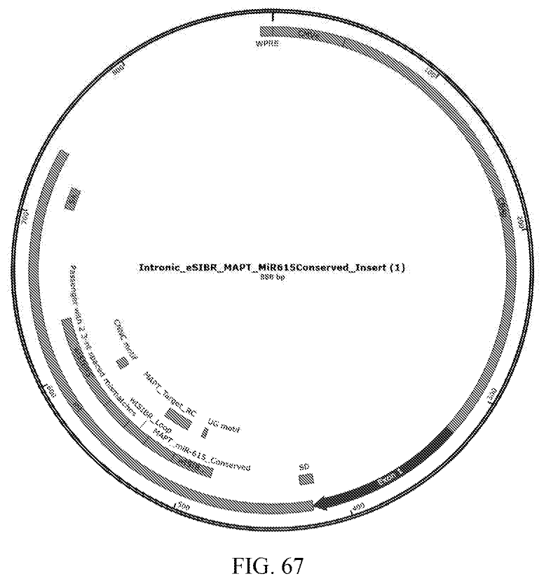

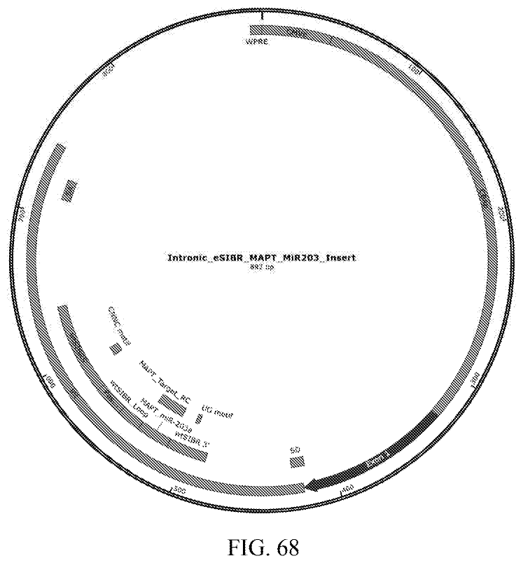

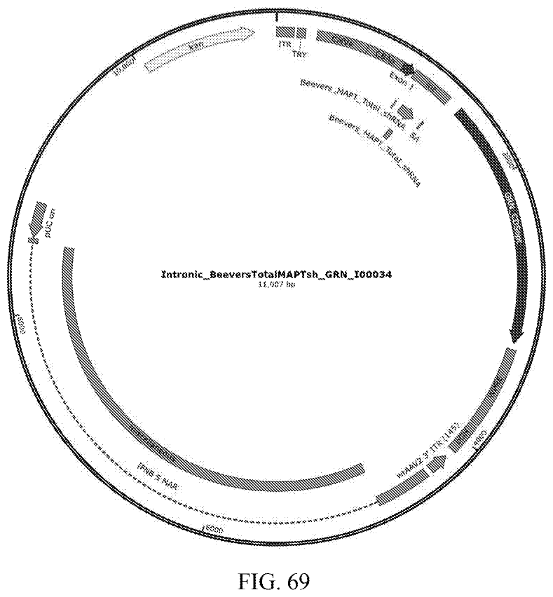

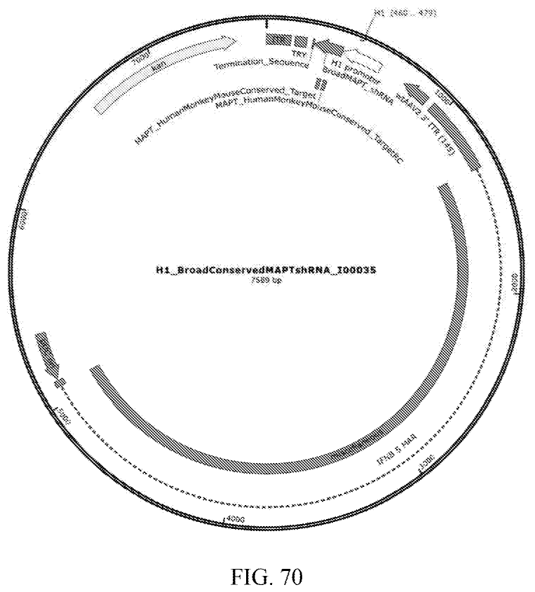

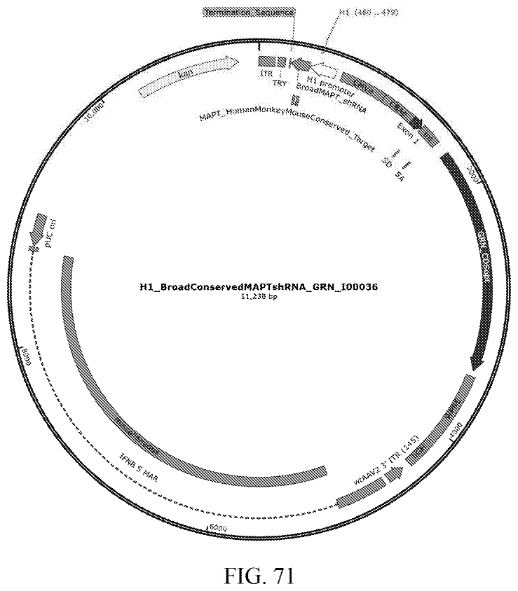

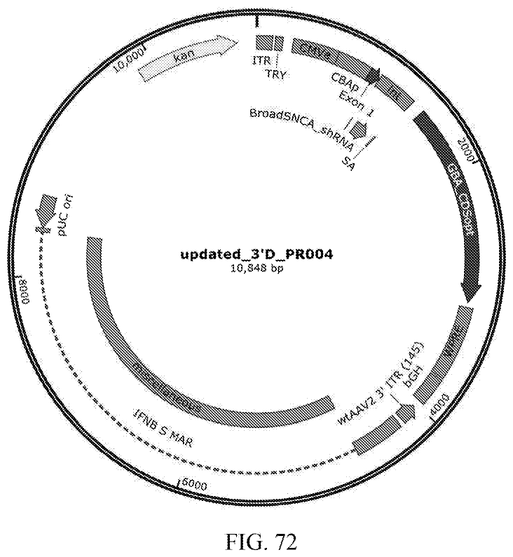

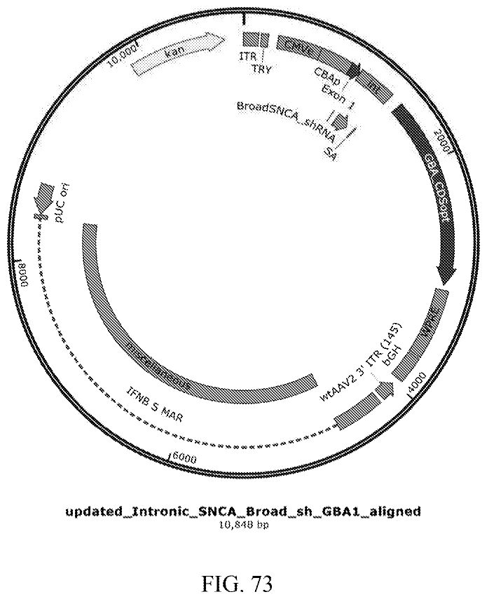

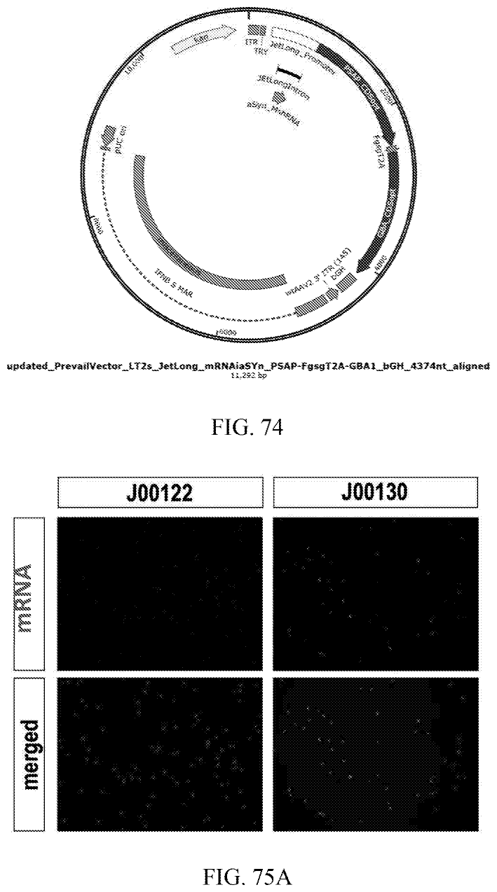

AAV vectors are generated using cells, such as HEK293 cells for triple-plasmid transfection. The ITR sequences flank an expression construct comprising a promoter/enhancer element for each transgene of interest, a 3′ polyA signal, and posttranslational signals such as the WPRE element. Multiple gene products can be expressed simultaneously such as GBA1 and LIMP2 and/or Prosaposin, by fusion of the protein sequences; or using a 2A peptide linker, such as T2A or P2A, which leads 2 peptide fragments with added amino acids due to prevention of the creation of a peptide bond; or using an IRES element; or by expression with 2 separate expression cassettes. The presence of a short intronic sequence that is efficiently spliced, upstream of the expressed gene, can improve expression levels. shRNAs and other regulatory RNAs can potentially be included within these sequences. Examples of expression constructs described by the disclosure are shown in FIGS. 1-8, 21-35, 39 and 41-51, and in Table 6 below.

TABLE 6

| | | | | | | | | | Length |
|---|---|---|---|---|---|---|---|---|---|
| | Promoter | | | | Bicistronic | Promoter | | | between |
| Name | 1 | shRNA | CDS1 | PolyA1 | element | 2 | CDS2 | PolyA2 | ITRs |
| CMVe_CBAp_GBA1_WPRE_bGH | CBA | | GBA1 | WPRE- | | | | | 3741 |
| | | | | bGH | | | | | |
| LT1s_JetLong_mRNAiaSYn_SCARB2-T2A-GBA1_bGH | JetLong | aSyn | SCARB2 | bGH | T2A | | GBA1 | | 4215 |
| LI1_JetLong_SCARB2-IRES-GBA1_bGH | JetLong | | SCARB2 | bGH | IRES | | GBA1 | | 4399 |
| FP1_JetLong_GBA1_bGH_JetLong_SCARB2_SV40L | JetLong | | GBA1 | bGH | | JetLong | SCARB2 | SV40L | 4464 |
| PrevailVector_LT2s_JetLong_mRNAiaSYn_PSAP-T2A-GBA1_bGH_4353nt | JetLong | aSyn | PSAP | bGH | T2A | — | GBA1 | — | 4353 |
| PrevailVector_LI2_JetLong_PSAP_IRES_GBA1_SymtheticpolyA_4337nt | JetLong | — | PSAP | Synthetic | IRES | — | GBA1 | — | 4337 |
| | | | | pA | | | | | |
| PrevailVector_10s_JetLong_mRNAiaSy_GBA2_WPRE_bGH_4308nt | JetLong | aSyn | GBA2 | WPRE_bGH | — | — | — | — | 4308 |
| PrevailVector_FT4_JetLong_GBA1_T2A_GALC_SyntheticpolyA_4373nt | JetLong | — | GBA1 | Synthetic | T2A | — | GALC | — | 4373 |
| | | | | pA | | | | | |
| PrevailVector_LT4_JetLong_GALC_T2A_GBA1_SyntheticpolyA_4373nt | JetLong | — | GALC | Synthetic | T2A | — | GBA1 | — | 4373 |
| | | | | pA | | | | | |
| PrevailVector_LT5s_JetLong_mRNAiaSyn_CTSB-T2A-GBA1_WPRE_bGH_4392nt | JetLong | aSyn | CTSB | WPRE_bGH | T2A | — | GBA1 | — | 4392 |
| PrevailVector_FT11t_JetLong_mRNAiaSyn_GBA1_T2S_SMPD1_SyntheticpolyA_4477nt | JetLong | aSyn | GBA1 | Synthetic | T2A | — | SMPD1 | — | 4477 |
| | | | | pA | | | | | |
| PrevailVector_LI4_JetLong_GALC_IRES_GBA1_SymtheticpolyA_4820nt | JetLong | — | GALC | Synthetic | IRES | — | GBA1 | — | 4820 |
| | | | | pA | | | | | |
| PrevailVector_FP5_JetLong_GBA1_bGH_JetLong_CTSB_SV401_4108nt | JetLong | — | GBA1 | bGH | — | JetLong | CTSB | SV40L | 4108 |
| PrevailVector_FT6s_JetLong_mRNAiaSyn_GBA1-T2A-GCH1_WPRE_bGH_4125nt | JetLong | aSyn | GBA1 | WPRE_bGH | T2A | — | GCH1 | — | 4125 |
| PrevailVector_LT7s_JetLong_mRNAiaSyn_RAB7L1-T2A-GBA1_WPRE_bGH_3984nt | JetLong | aSyn | RAB7L1 | WPRE_bGH | T2A | — | GBA1 | — | 3984 |
| PrevailVector_FI6s_JetLong_mRNAiaSYn_GBA1-IRES-GCH1_bGH_3978nt | JetLong | aSyn | GBA1 | bGH | IRES | — | GCH1 | — | 3978 |
| PrevailVector_9st_JetLong_mRNAiaSyn_mRNAiTMEM106B_VPS35_WPRE_bGH_4182nt | JetLong | aSyn | VPS35 | WPRE_bGH | — | — | — | — | 4182 |
| | | & | | | | | | | |
| | | TMEM106B | | | | | | | |
| PrevailVector_FT12s_JetLong_mRNAiaSyn_GBA1-T2A-IL34_WPRE_bGH_4104nt | JetLong | aSyn | GBA1 | WPRE_bGH | T2A | — | IL34 | — | 4104 |
| PrevailVector_FI12s_JetLong_mRNAiaSYn_GBA1-IRES-IL34_bGH_3957nt | JetLong | aSyn | GBA1 | bGH | IRES | — | IL34 | — | 3957 |
| PrevailVector_FP8_JetLong_GBA1_bGH_CD68_TREM2_SV401_4253nt | JetLong | — | GBA1 | bGH | — | CD68 | TREM2 | SV40L | 4253 |
| PrevailVector_FP12_CMVe_CBA_GBA1_bGH_JetLong_IL34_SV40l_4503nt | CBA | | GBA1 | bGH | | JetLong | IL34 | SV40L | 4503 |
| PrevailVector_0_CMVe_CBAp_mRNAiaSyn_GBA1_WPRE_bGH_4004nt | CBA | aSyn | GBA1 | WPRE_bGH | — | — | — | — | 4004 |
| PrevailVector_X1_SNCA | CMVe + | — | SNCA | WPRE_bGH | — | — | — | — | — |
| | CBA | | | | | | | | |

### Example 2: Cell Based Assays of Viral Transduction into GBA-Deficient Cells

Cells deficient in GBA1 are obtained, for example as fibroblasts from GD patients, monocytes, or hES cells, or patient-derived induced pluripotent stem cells (iPSCs). These cells accumulate substrates such as glucosylceramide and glucosylsphingosine (GlcCer and GlcSph). Treatment of wild-type or mutant cultured cell lines with Gcase inhibitors, such as CBE, is also be used to obtain GBA deficient cells.

Using such cell models, lysosomal defects are quantified in terms of accumulation of protein aggregates, such as of α-Synuclein with an antibody for this protein or phospho-αSyn, followed by imaging using fluorescent microscopy. Imaging for lysosomal abnormalities by ICC for protein markers such as LAMP1, LAMP2, LIMP1, LIMP2, or using dyes such as Lysotracker, or by uptake through the endocytic compartment of fluorescent dextran or other markers is also performed. Imaging for autophagy marker accumulation due to defective fusion with the lysosome, such as for LC3, can also be performed. Western blotting and/or ELISA is used to quantify abnormal accumulation of these markers. Also, the accumulation of glycolipid substrates and products of GBA1 is measured using standard approaches.

Therapeutic endpoints (e.g., reduction of PD-associated pathology) are measured in the context of expression of transduction of the AAV vectors, to confirm and quantify activity and function. Gcase can is also quantified using protein ELISA measures, or by standard Gcase activity assays.

### Example 3: In Vivo Assays Using Mutant Mice

This example describes in vivo assays of AAV vectors using mutant mice. In vivo studies of AAV vectors as above in mutant mice are performed using assays described, for example, by Liou et al. (2006) J. Biol. Chem. 281(7): 4242-4253, Sun et al. (2005) J. Lipid Res. 46:2102-2113, and Farfel-Becker et al. (2011) Dis. Model Mech. 4(6):746-752.

The intrathecal or intraventricular delivery of vehicle control and AAV vectors (e.g., at a dose of 2×10¹¹ vg/mouse) are performed using concentrated AAV stocks, for example at an injection volume between 5-10 μL. Intraparenchymal delivery by convection enhanced delivery is performed.

Treatment is initiated either before onset of symptoms, or subsequent to onset. Endpoints measured are the accumulation of substrate in the CNS and CSF, accumulation of Gcase enzyme by ELISA and of enzyme activity, motor and cognitive endpoints, lysosomal dysfunction, and accumulation of α-Synuclein monomers, protofibrils or fibrils.

### Example 4: Chemical Models of Disease

This example describes in vivo assays of AAV vectors using a chemically-induced mouse model of Gaucher disease (e.g., the CBE mouse model). In vivo studies of these AAV vectors are performed in a chemically-induced mouse model of Gaucher disease, for example as described by Vardi et al. (2016) J Pathol. 239(4):496-509.

Intrathecal or intraventricular delivery of vehicle control and AAV vectors (e.g., at a dose of 2×10¹¹ vg/mouse) are performed using concentrated AAV stocks, for example with injection volume between 5-10 μL. Intraparenchymal delivery by convection enhanced delivery is performed. Peripheral delivery is achieved by tail vein injection.

Treatment is initiated either before onset of symptoms, or subsequent to onset. Endpoints measured are the accumulation of substrate in the CNS and CSF, accumulation of Gcase enzyme by ELISA and of enzyme activity, motor and cognitive endpoints, lysosomal dysfunction, and accumulation of α-Synuclein monomers, protofibrils or fibrils.

### Example 5: Clinical Trials in PD, LBD, Gaucher Disease Patients

In some embodiments, patients having certain forms of Gaucher disease (e.g., GD1) have an increased risk of developing Parkinson's disease (PD) or Lewy body dementia (LBD). This Example describes clinical trials to assess the safety and efficacy of rAAVs as described by the disclosure, in patients having Gaucher disease, PD and/or LBD.

Clinical trials of such vectors for treatment of Gaucher disease, PD and/or LBD are performed using a study design similar to that described in Grabowski et al. (1995) Ann. Intern. Med. 122(1):33-39.

### Example 6: Treatment of Peripheral Disease

In some embodiments, patients having certain forms of Gaucher disease exhibit symptoms of peripheral neuropathy, for example as described in Biegstraaten et al. (2010) Brain 133(10):2909-2919.

This example describes in vivo assays of AAV vectors as described herein for treatment of peripheral neuropathy associated with Gaucher disease (e.g., Type 1 Gaucher disease). Briefly, Type 1 Gaucher disease patients identified as having signs or symptoms of peripheral neuropathy are administered a rAAV as described by the disclosure. In some embodiments, the peripheral neuropathic signs and symptoms of the subject are monitored, for example using methods described in Biegstraaten et al., after administration of the rAAV.

Levels of transduced gene products as described by the disclosure present in patients (e.g., in serum of a patient, in peripheral tissue (e.g., liver tissue, spleen tissue, etc.)) of a patient are assayed, for example by Western blot analysis, enzymatic functional assays, or imaging studies.

### Example 7: Treatment of CNS forms

This example describes in vivo assays of rAAVs as described herein for treatment of CNS forms of Gaucher disease. Briefly, Gaucher disease patients identified as having a CNS form of Gaucher disease (e.g., Type 2 or Type 3 Gaucher disease) are administered a rAAV as described by the disclosure. Levels of transduced gene products as described by the disclosure present in the CNS of patients (e.g., in serum of the CNS of a patient, in cerebrospinal fluid (CSF) of a patient, or in CNS tissue of a patient) are assayed, for example by Western blot analysis, enzymatic functional assays, or imaging studies.

### Example 8: Gene Therapy of Parkinson's Disease in Subjects Having Mutations in GBA1

This example describes administration of a recombinant adeno-associated virus (rAAV) encoding GBA1 to a subject having Parkinson's disease characterized by a mutation in GBA1gene.

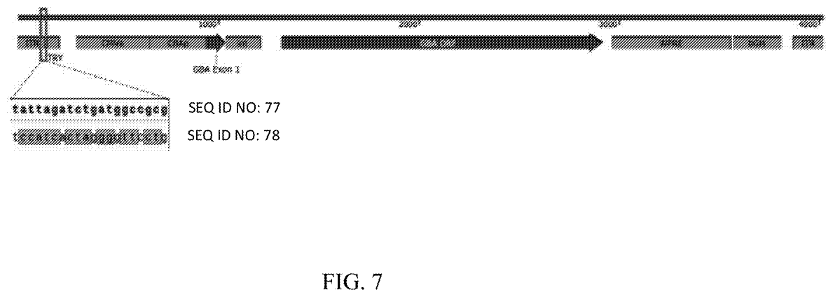

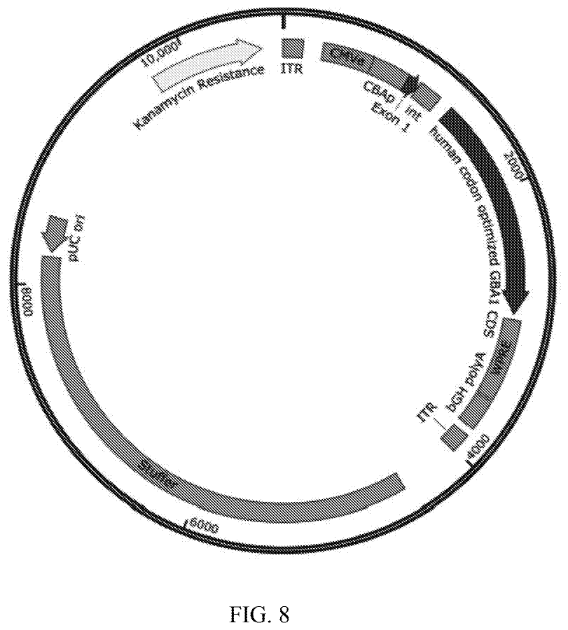

The rAAV-GBA1 vector insert contains the CBA promoter element (CBA), consisting of four parts: the CMV enhancer (CMVe), CBA promoter (CBAp), Exon 1, and intron (int) to constitutively express the codon optimized coding sequence (CDS) of human GBA1 (maroon). The 3′ region also contains a Woodchuck hepatitis virus Posttranscriptional Regulatory Element (WPRE) posttranscriptional regulatory element followed by a bovine Growth Hormone polyA signal (bGH polyA) tail. The flanking ITRs allow for the correct packaging of the intervening sequences. Two variants of the 5′ ITR sequence (FIG. 7, inset box, bottom sequence) were evaluated; these variants have several nucleotide differences within the 20-nucleotide “D” region of the ITR, which is believed to impact the efficiency of packaging and expression. The rAAV-GBA1 vector product contains the “D” domain nucleotide sequence shown in FIG. 7 (inset box, top sequence). A variant vector harbors a mutant “D” domain (termed an “S” domain herein, with the nucleotide changes shown by shading), performed similarly in preclinical studies. The backbone contains the gene to confer resistance to kanamycin as well as a stuffer sequence to prevent reverse packaging. A schematic depicting a rAAV-GBA1 vector is shown in FIG. 8. The rAAV-GBA1 vector is packaged into an rAAV using AAV9 serotype capsid proteins.

rAAV-GBA1 is administered to a subject as a single dose via a fluoroscopy guided sub-occipital injection into the cisterna magna (intracisternal magna; ICM). One embodiment of a rAAV-GBA1 dosing regimen study is as follows:

A single dose of rAAV-GBA1 is administered to patients (N=12) at one of two dose levels (3 e13 vg (low dose); 1 e14 vg (high dose), etc.) which are determined based on the results of nonclinical pharmacology and toxicology studies.

Initial studies were conducted in a chemical mouse model involving daily delivery of conduritol-b-epoxide (CBE), an inhibitor of GCase to assess the efficacy and safety of the rAAV-GBA1 vector and a rAAV-GBA1 S-variant construct (as described further below). Additionally, initial studies were performed in a genetic mouse model, which carries a homozygous GBA1 mutation and is partially deficient in saposins (4L/PS-NA). Additional dose-ranging studies in mice and nonhuman primates (NHPs) are conducted to further evaluate vector safety and efficacy.

Two slightly different versions of the 5′ inverted terminal repeat (ITR) in the AAV backbone were tested to assess manufacturability and transgene expression (FIG. 7). The 20 bp “D” domain within the 145 bp 5′ ITR is thought to be necessary for optimal viral vector production, but mutations within the “D” domain have also been reported to increase transgene expression in some cases. Thus, in addition to the viral vector rAAV-GBA1, which harbors an intact “D” domain, a second vector form with a mutant D domain (termed an “S” domain herein) was also evaluated. Both rAAV-GBA1 and the variant express the same transgene. While both vectors produced virus that was efficacious in vivo as detailed below, rAAV-GBA1, which contains a wild-type “D” domain, was selected for further development.

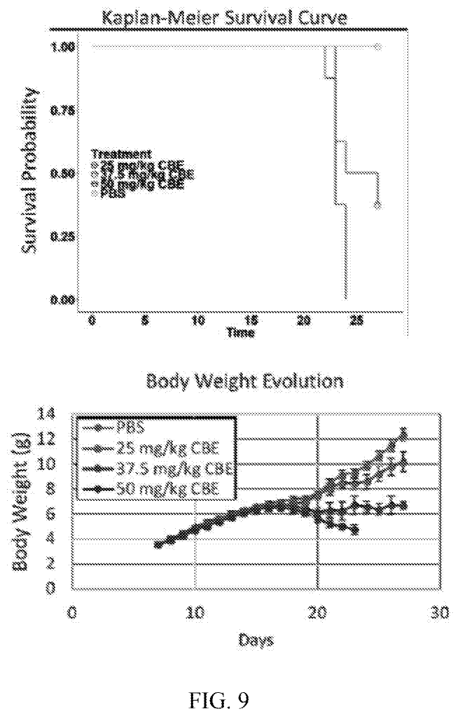

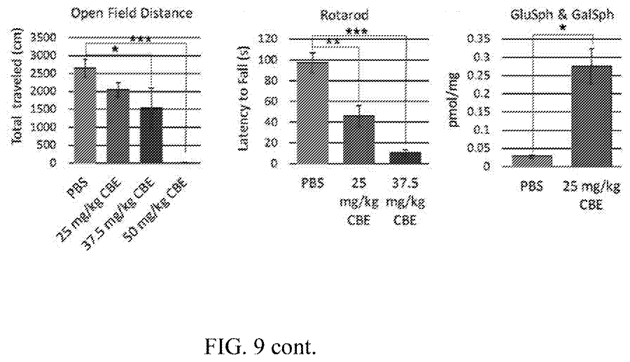

To establish the CBE model of GCase deficiency, juvenile mice were dosed with CBE, a specific inhibitor of GCase. Mice were given CBE by IP injection daily, starting at postnatal day 8 (P8). Three different CBE doses (25 mg/kg, 37.5 mg/kg, 50 mg/kg) and PBS were tested to establish a model that exhibits a behavioral phenotype (FIG. 9). Higher doses of CBE led to lethality in a dose-dependent manner. All mice treated with 50 mg/kg CBE died by P23, and 5 of the 8 mice treated with 37.5 mg/kg CBE died by P27. There was no lethality in mice treated with 25 mg/kg CBE. Whereas CBE-injected mice showed no general motor deficits in the open field assay (traveling the same distance and at the same velocity as mice given PBS), CBE-treated mice exhibited a motor coordination and balance deficit as measured by the rotarod assay.

Mice surviving to the end of the study were sacrificed on the day after their last CBE dose (P27, “Day 1”) or after three days of CBE withdrawal (P29, “Day 3”). Lipid analysis was performed on the cortex of mice given 25 mg/kg CBE to evaluate the accumulation of GCase substrates in both the Day 1 and Day 3 cohorts. GluSph and GalSph levels (measured in aggregate in this example) were significantly accumulated in the CBE-treated mice compared to PBS-treated controls, consistent with GCase insufficiency.

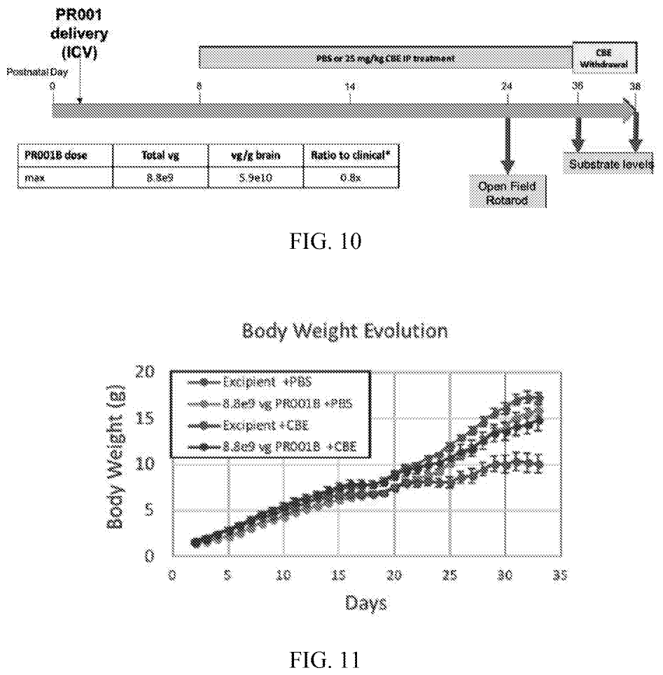

Based on the study described above, the 25 mg/kg CBE dose was selected since it produced behavioral deficits without impacting survival. To achieve widespread GBA1 distribution throughout the brain and transgene expression during CBE treatment, rAAV-GBA1 or excipient was delivered by intracerebroventricular (ICV) injection at postnatal day 3 (P3) followed by daily IP CBE or PBS treatment initiated at P8 (FIG. 10).

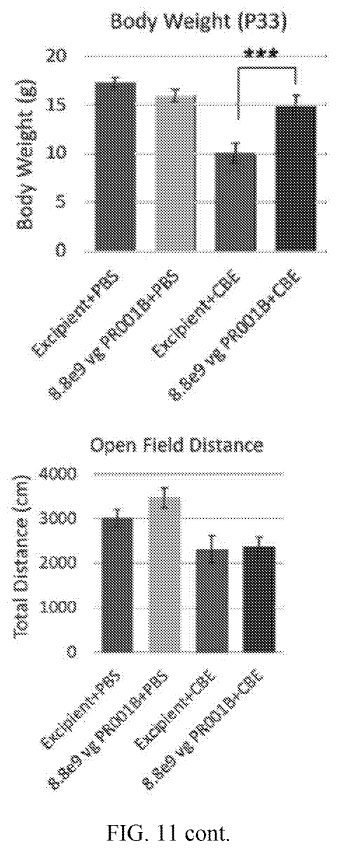

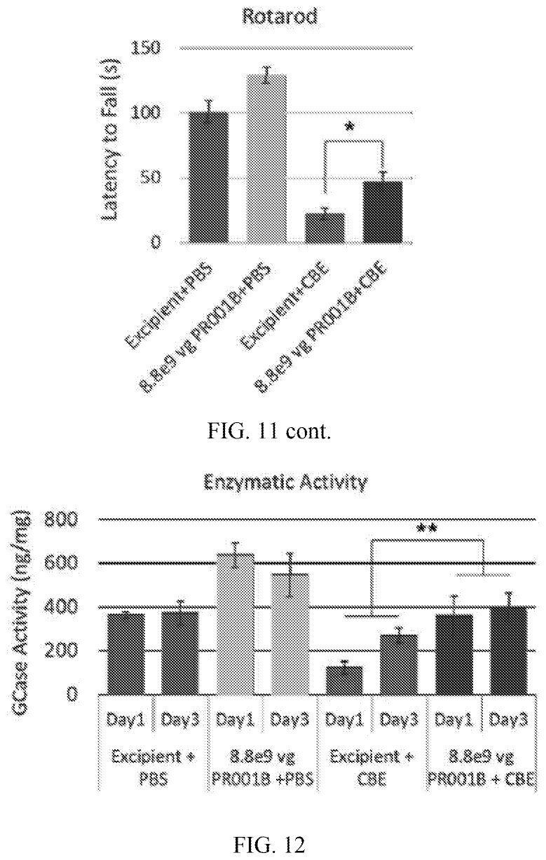

CBE-treated mice that received rAAV-GBA1 performed statistically significantly better on the rotarod than those that received excipient (FIG. 11). Mice in the variant treatment group did not differ from excipient treated mice in terms of other behavioral measures, such as the total distance traveled during testing (FIG. 11).

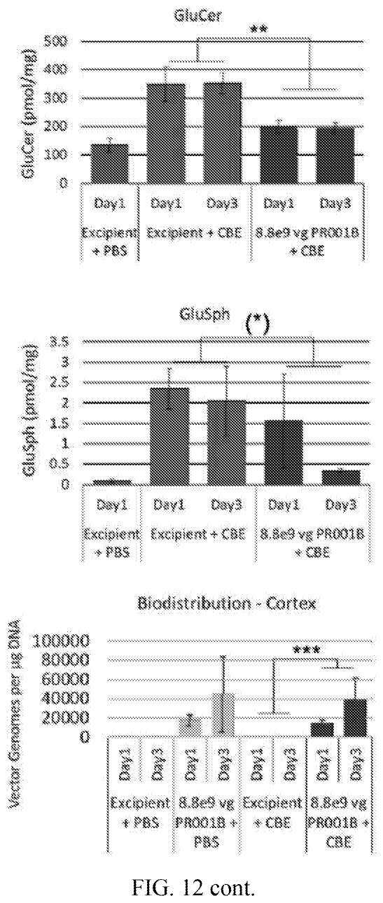

At the completion of the in-life study, half of the mice were sacrificed the day after the last CBE dose (P36, “Day 1”) or after three days of CBE withdrawal (P38, “Day 3”) for biochemical analysis (FIG. 12). Using a fluorometric enzyme assay performed in biological triplicate, GCase activity was assessed in the cortex. GCase activity was increased in mice that were treated with rAAV-GBA1, while CBE treatment reduced GCase activity. Additionally, mice that received both CBE and rAAV-GBA1 had GCase activity levels that were similar to the PBS-treated group, indicating that delivery of rAAV-GBA1 is able to overcome the inhibition of GCase activity induced by CBE treatment. Lipid analysis was performed on the motor cortex of the mice to examine levels of the substrates GluCer and GluSph. Both lipids accumulated in the brains of mice given CBE, and rAAV-GBA1 treatment significantly reduced substrate accumulation.

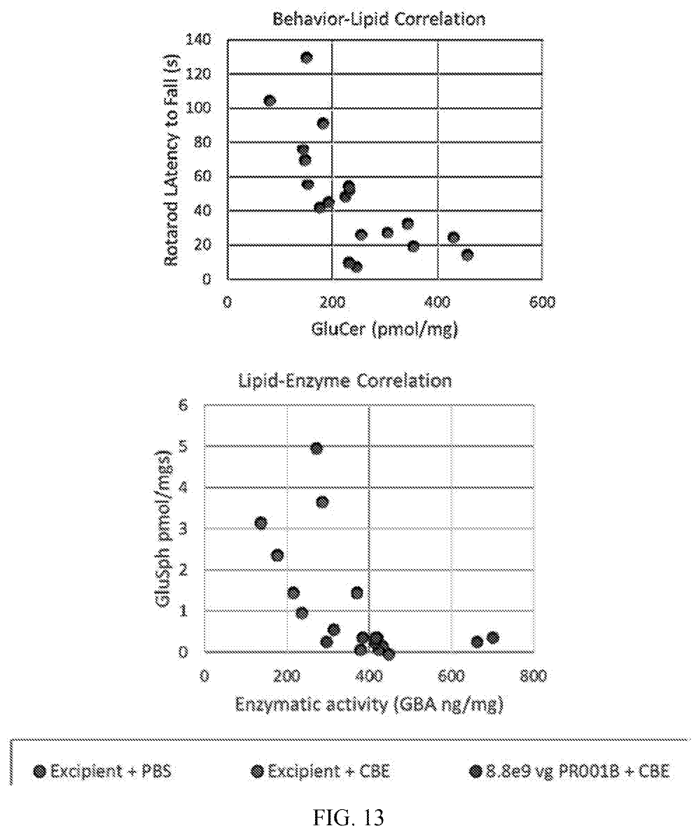

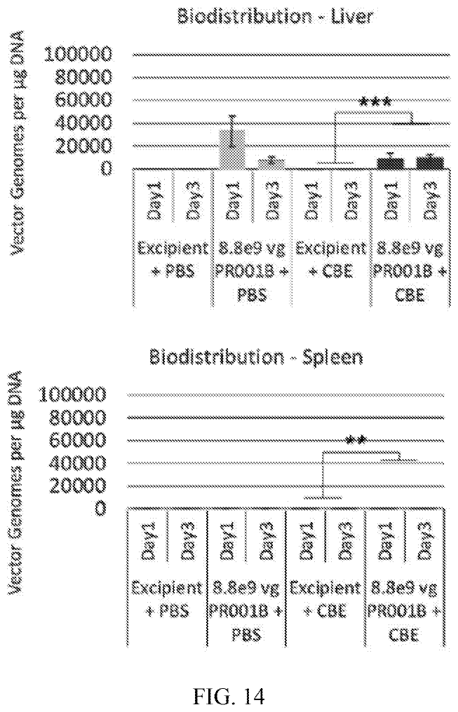

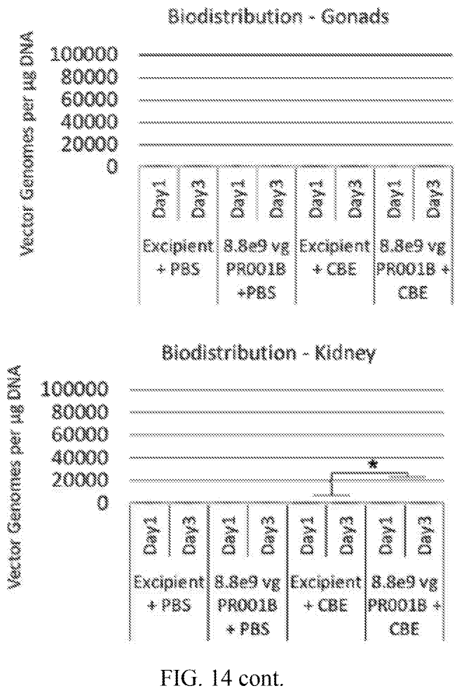

Lipid levels were negatively correlated with both GCase activity and performance on the Rotarod across treatment groups. The increased GCase activity after rAAV-GBA1 administration was associated with substrate reduction and enhanced motor function (FIG. 13). As shown in FIG. 14, preliminary biodistribution was assessed by vector genome presence, as measured by qPCR (with >100 vector genomes per 1 μg genomic DNA defined as positive). Mice that received rAAV-GBA1, both with and without CBE, were positive for rAAV-GBA1 vector genomes in the cortex, indicating that ICV delivery results in rAAV-GBA1 delivery to the cortex. Additionally, vector genomes were detected in the liver, few in spleen, and none in the heart, kidney or gonads. For all measures, there was no statistically significant difference between the Day 1 and Day 3 groups.

A larger study in the CBE model further explored efficacious doses of rAAV-GBA1 in the CBE model. Using the 25 mg/kg CBE dose model, excipient or rAAV-GBA1 was delivered via ICV at P3, and daily IP PBS or CBE treatment initiated at P8. Given the similarity between the groups with and without CBE withdrawal observed in the previous studies, all mice were sacrificed one day after the final CBE dose (P38-40). The effect of three different rAAV-GBA1 doses was assessed, resulting in the following five groups, with 10 mice (5M/5F) per group:
Excipient ICV+PBS IP
Excipient ICV+25 mg/kg CBE IP
3.2 e9 vg (2.13 e10 vg/g brain) rAAV-GBA1 ICV+25 mg/kg CBE IP
1.0 e10 vg (6.67 e10 vg/g brain) rAAV-GBA1 ICV+25 mg/kg CBE IP
3.2 e10 vg (2.13 e11 vg/g brain) rAAV-GBA1 ICV+25 mg/kg CBE IP.

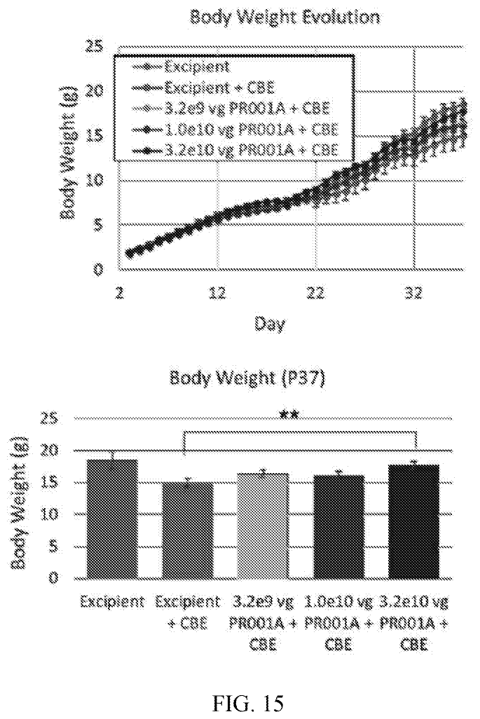

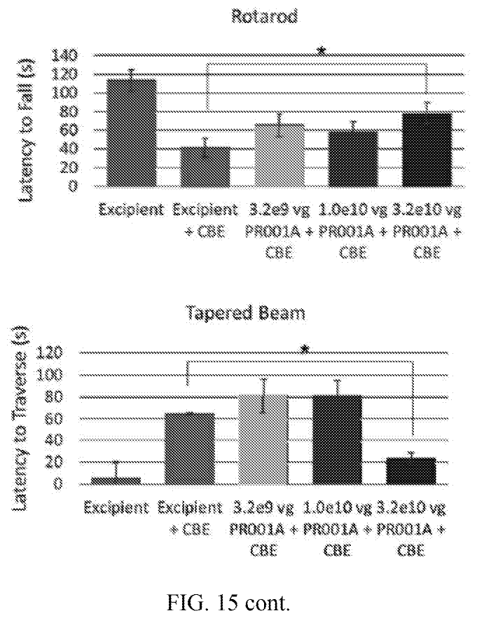

The highest dose of rAAV-GBA1 rescued the CBE treatment-related failure to gain weight at P37. Additionally, this dose resulted in a statistically significant increase in performance on the rotarod and tapered beam compared to the Excipient+CBE treated group (FIG. 15). Lethality was observed in several groups, including both excipient-treated and rAAV-GBA1-treated groups (Excipient+PBS: 0; Excipient+25 mg/kg CBE: 1; 3.2 e9 vg rAAV-GBA1+25 mg/kg CBE: 4; 1.0 e10 vg rAAV-GBA1+25 mg/kg CBE: 0; 3.2 e10 vg rAAV-GBA1+25 mg/kg CBE: 3).

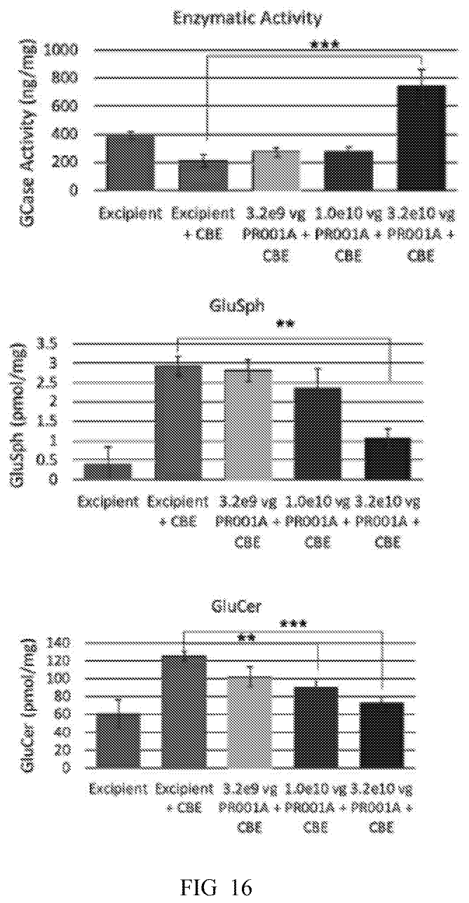

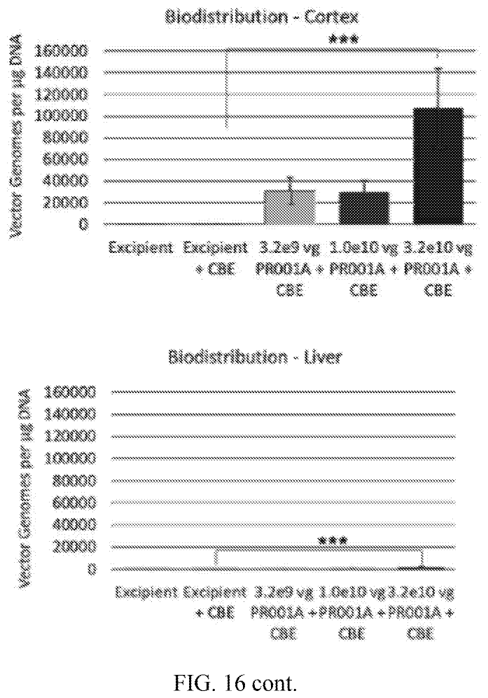

At the completion of the in-life study, mice were sacrificed for biochemical analysis (FIG. 16). GCase activity in the cortex was assessed in biological triplicates by a fluorometric assay. CBE-treated mice showed reduced GCase activity whereas mice that received a high rAAV-GBA1 dose showed a statistically significant increase in GCase activity compared to CBE treatment. CBE-treated mice also had accumulation of GluCer and GluSph, both of which were rescued by administering a high dose of rAAV-GBA1.

In addition to the established chemical CBE model, rAAV-GBA1 is also evaluated in the 4L/PS-NA genetic model, which is homozygous for the V394L GD mutation in Gbal and is also partially deficient in saposins, which affect GCase localization and activity. These mice exhibit motor strength, coordination, and balance deficits, as evidenced by their performance in the beam walk, rotarod, and wire hang assays. Typically the lifespan of these mice is less than 22 weeks. In an initial study, 3 μl of maximal titer virus was delivered by ICV at P23, with a final dose of 2.4 e10 vg (6.0 e10 vg/g brain). With 6 mice per group, the treatment groups were:
WT+Excipient ICV
4L/PS-NA+Excipient ICV
4L/PS-NA+2.4 e10 vg (6.0 e10 vg/g brain) rAAV-GBA1 ICV

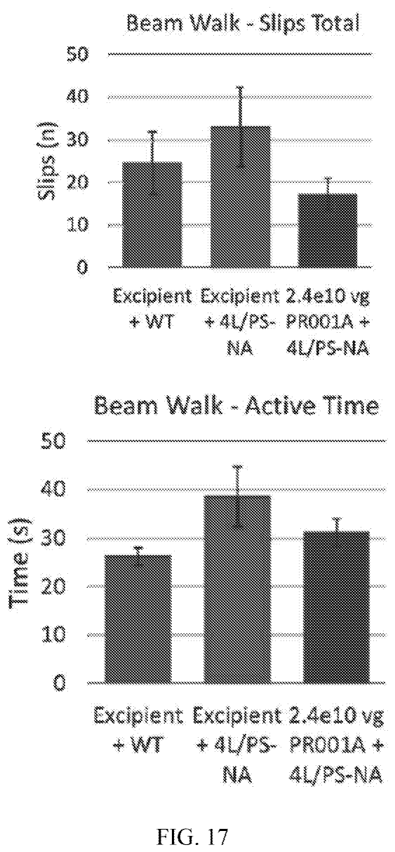

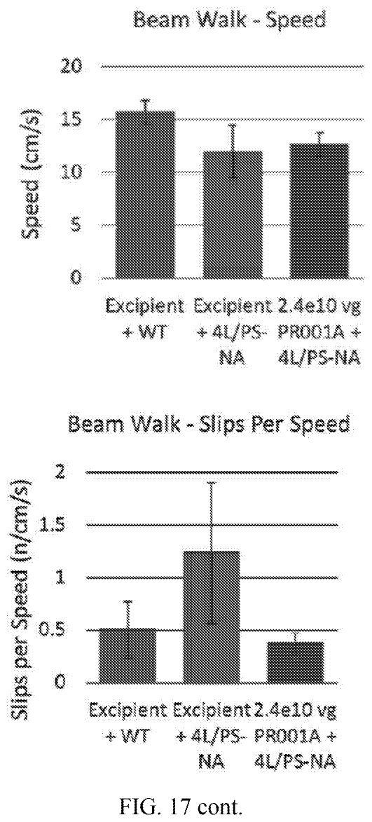

Motor performance by the beam walk test was assessed 4 weeks post-rAAV-GBA1 delivery. The group of mutant mice that received rAAV-GBA1 showed a trend towards fewer total slips and fewer slips per speed when compared to mutant mice treated with excipient, restoring motor function to near WT levels (FIG. 17). Since the motor phenotypes become more severe as these mice age, their performance on this and other behavioral tests is assessed at later time points. At the completion of the in-life study, lipid levels, GCase activity, and biodistribution are assessed in these mice.

Additional lower doses of rAAV-GBA1 are currently being tested using the CBE model, corresponding to 0.03×, 0.1×, and 1× the proposed phase 1 high clinical dose. Each group includes 10 mice (5M/5F) per group:
Excipient ICV
Excipient ICV+25 mg/kg CBE IP
3.2 e8 vg (2.13 e9 vg/g brain) rAAV-GBA1 ICV+25 mg/kg CBE IP
1.0 e9 vg (6.67 e9 vg/g brain) rAAV-GBA1 ICV+25 mg/kg CBE IP
1.0 e10 vg (6.67 e10 vg/g brain) rAAV-GBA1 ICV+25 mg/kg CBE IP.

In addition to motor phenotypes, lipid levels and GCase activity are assessed in the cortex. Time course of treatments and analyses are also performed.

A larger dose ranging study was initiated to evaluate efficacy and safety data. 10 4L/PS-NA mice (5M/5F per group) were injected with 10 μl of rAAV-GBA1. Using an allometric brain weight calculation, the doses correlate to 0.15×, 1.5×, 4.4×, and 14.5× the proposed phase 1 high clinical dose. The injection groups consist of:
WT+Excipient ICV
4L/PS-NA+Excipient ICV
4L/PS-NA+4.3 e9 vg (1.1 e10 vg/g brain) rAAV-GBA1 ICV
4L/PS-NA+4.3 e10 vg (1.1 e11 vg/g/brain) rAAV-GBA1 ICV
4L/PS-NA+1.3 e11 vg (3.2 e11 vg/g brain) rAAV-GBA1 ICV
4L/PS-NA+4.3 e11 vg (1.1 e12 vg/g brain) rAAV-GBA1 ICV.

A summary of nonclinical studies in the CBE model are shown in Table 7 below.

TABLE 7
Summary of Results in CBE Mouse Model

| | | | Behavioral Changes | | | | | | |
|---|---|---|---|---|---|---|---|---|---|
| Test | Study | | | Tapered | Open | | | BD | |
| Material | Number | Dose Cohort | Rotarod | Beam | Field | Lipids | Enzyme | Brain | Liver |
| rAAV- | PRV-2018- | 3.2e9 vg | NS | NS | NS | NS | NS | + | − |
| GBA1 | 005 Dose- | (2.13e10 | | | | | | | |
| | ranging | vg/g brain) | | | | | | | |
| | rAAV- | 1.10e10 vg | T | NS | NS | T/S | NS | + | + |
| | GBA1 in | (6.67e10 | | | | | | | |
| | CBE Model | vg/g brain) | | | | | | | |
| | | 2.3e10 vg | S | S | NS | S | S | + | + |
| | | (2.13e11 | | | | | | | |
| | | vg/g brain) | | | | | | | |
| Variant | PRV-2018- | 8.8e9 vg | S | N/A | NS | S | S | + | + |
| | 005 Dose- | (5.9e10 | | | | | | | |
| | ranging | vg/g brain) | | | | | | | |
| | Variant in | | | | | | | | |
| | CBE Model | | | | | | | | |

Note that positive biodistribution is defined as >100 vg/1 μg genomic DNA.
Abbreviations: BD = biodistribution; NS = nonsignificant; T = trend; S = significant; N/A = not applicable; + = positive; − = negative.

### Example 9: In Vitro Analysis of rAAV Vectors

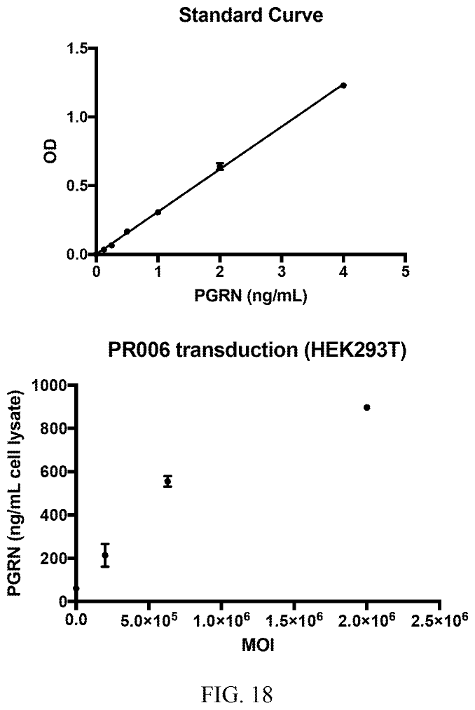

rAAV constructs were tested in vitro and in vivo. FIG. 18 shows representative data for in vitro expression of rAAV constructs encoding progranulin (PGRN, also referred to as GRN) protein. The left panel shows a standard curve of progranulin (PGRN) ELISA assay. The bottom panel shows a dose-response of PGRN expression measured by ELISA assay in cell lysates of HEK293T cells transduced with rAAV. MOI=multiplicity of infection (vector genomes per cell).

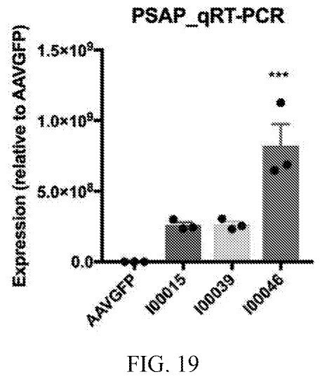

A pilot study was performed to assess in vitro activity of rAAV vectors encoding Prosaposin (PSAP) and SCARB2, alone or in combination with GBA1 and/or one or more inhibitory RNAs. One construct encoding PSAP and progranulin (PGRN, also referred to as GRN) was also tested. Vectors tested include those shown in Table 4. “Opt” refers to a nucleic acid sequence codon optimized for expression in mammalian cells (e.g., human cells). FIG. 19 shows representative data indicating that transfection of HEK293 cells with each of the constructs resulted in overexpression of the corresponding gene product compared to mock transfected cells.

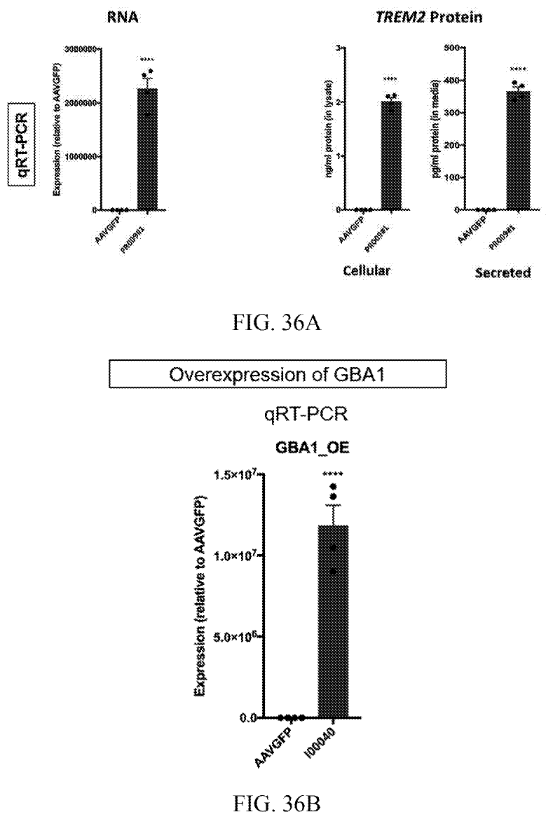

A pilot study was performed to assess in vitro activity of rAAV vectors encoding TREM2, alone or in combination with one or more inhibitory RNAs. Vectors tested include those shown in Table 8. “Opt” refers to a nucleic acid sequence codon optimized for expression in mammalian cells (e.g., human cells). FIGS. 36A-36B show representative data indicating that transfection of HEK293 cells with each of the constructs resulted in overexpression of the corresponding gene product compared to mock transfected cells.

TABLE 8

| ID | Promoter | Inhibitory RNA | Promoter | Transgene |
|---|---|---|---|---|
| I00015 | JL_intronic | SNCA | JetLong | Opt- |
| | | | | PSAP_GBA1 |
| I00039 | — | — | JetLong | Opt-PSAP-GRN |
| I00046 | — | — | CBA | Opt-PSAP |
| I00014 | JetLong | SNCA | JetLong | Opt- |
| | | | | SCARB2_GBA1 |
| I00040 | | | JL, CD68 | opt-GBA1, |
| | | | | TREM2 |

### Example 10: Testing of SNCA and TMEM106B shRNA Constructs HEK293 Cells

Human embryonic kidney 293 cell line (HEK293) were used in this study (#85120602, Sigma-Aldrich). HEK293 cells were maintained in culture media (D-MEM [#11995065, Thermo Fisher Scientific] supplemented with 10% fetal bovine serum [FBS] [#10082147, Thermo Fisher Scientific]) containing 100 units/ml penicillin and 100 m/ml streptomycin (#15140122, Thermo Fisher Scientific).

### Plasmid Transfection

Plasmid transfection was performed using Lipofectamine 2000 transfection reagent (#11668019, Thermo Fisher Scientific) according to the manufacture's instruction. Briefly, HEK293 cells (#12022001, Sigma-Aldrich) were plated at the density of 3×10⁵ cells/ml in culture media without antibiotics. On the following day, the plasmid and Lipofectamine 2000 reagent were combined in Opti-MEM solution (#31985062, Thermo Fisher Scientific). After 5 minutes, the mixtures were added into the HEK293 culture. After 72 hours, the cells were harvested for RNA or protein extraction, or subjected to the imaging analyses. For imaging analyses, the plates were pre-coated with 0.01% poly-L-Lysine solution (P8920, Sigma-Aldrich) before the plating of cells.

Gene Expression Analysis by Quantitative Real-Time PCR (qRT-PCR)

Relative gene expression levels were determined by quantitative real-time PCR (qRT-PCR) using Power SYBR Green Cells-to-CT Kit (#4402955, Thermo Fisher Scientific) according to the manufacturer's instruction. The candidate plasmids were transiently transfected into HEK293 cells plated on 48-well plates (7.5×10⁴ cells/well) using Lipofectamine 2000 transfection reagent (0.5 m plasmid and 1.5 μl reagent in 50 μl Opti-MEM solution). After 72 hours, RNA was extracted from the cells and used for reverse transcription to synthesize cDNA according to the manufacturer's instruction. For quantitative PCR analysis, 2-5 μl of cDNA products were amplified in duplicates using gene specific primer pairs (250 nM final concentration) with Power SYBR Green PCR Master Mix (#4367659, Thermo Fisher Scientific). The primer sequences for SNCA, TMEM106B, and GAPDH genes were: 5′-AAG AGG GTG TTC TCT ATG TAG GC-3′ (SEQ ID NO: 71), 5′-GCT CCT CCA ACA TTT GTC ACT T-3′ (SEQ ID NO: 72) for SNCA, 5′-ACA CAG TAC CTA CCG TTA TAG CA-3′ (SEQ ID NO: 73), 5′-TGT TGT CAC AGT AAC TTG CAT CA-3′ (SEQ ID NO: 74) for TMEM106B, and 5′-CTG GGC TAC ACT GAG CAC C-3′ (SEQ ID NO: 75), 5′-AAG TGG TCG TTG AGG GCA ATG-3′ (SEQ ID NO: 76) for GAPDH. Quantitative PCR was performed in a QuantStudio 3 Real-Time PCR system (Thermo Fisher Scientific). Expression levels were normalized by the housekeeping gene GAPDH and calculated using the comparative CT method.

### Fluorescence Imaging Analysis

EGFP reporter plasmids, which contain 3′-UTR of human SNCA gene at downstream of EGFP coding region, were used for the validation of SNCA and TMEM106B knockdown plasmids. EGFP reporter plasmids and candidate knockdown plasmids were simultaneously transfected into HEK293 cells plated on poly-L-Lysine coated 96-well plates (3.0×10⁴ cells/well) using Lipofectamine 2000 transfection reagent (0.04 m reporter plasmid, 0.06 m knockdown plasmid and 0.3 μl reagent in 10 μl Opti-MEM solution). After 72 hours, the fluorescent intensities of EGFP signal were measured at excitation 488 nm/emission 512 nm using Varioskan LUX multimode reader (Thermo Fisher Scientific). Cells were fixed with 4% PFA at RT for 10 minutes, and incubated with D-PBS containing 40 m/ml 7-aminoactinomycin D (7-AAD) for 30 min at RT. After washing with D-PBS, the fluorescent intensities of 7-AAD signal were measured at excitation 546 nm/emission 647 nm using Varioskan reader to quantify cell number. Normalized EGFP signal per 7-AAD signal levels were compared with the control knockdown samples.

### Enzyme-Linked Immunosorbent Assay (ELISA)

α-Synuclein reporter plasmids, which contain 3′-UTR of human SNCA gene or TMEM106B gene downstream of SNCA coding region, were used for the validation of knockdown plasmids at the protein level. Levels of α-synuclein protein were determined by ELISA (#KHB0061, Thermo Fisher Scientific) using the lysates extracted from HEK293 cells. The candidate plasmids were transiently transfected into HEK293 cells plated on 48-well plates (7.5×10⁴ cells/well) using Lipofectamine 2000 transfection reagent (0.1 m reporter plasmid, 0.15 μg knockdown plasmid and 0.75 μl reagent in 25 μl Opti-MEM solution). After 72 hours, cells were lysed in radioimmunoprecipitation assay (RIPA) buffer (#89900, Thermo Fisher Scientific) supplemented with protease inhibitor cocktail (#P8340, Sigma-Aldrich), and sonicated for a few seconds. After incubation on ice for 30 min, the lysates were centrifuged at 20,000×g at 4° C. for 15 min, and the supernatant was collected. Protein levels were quantified. Plates were read in a Varioskan plate reader at 450 nm, and concentrations were calculated using SoftMax Pro 5 software. Measured protein concentrations were normalized to total protein concentration determined with a bicinchoninic acid assay (#23225, Thermo Fisher Scientific).

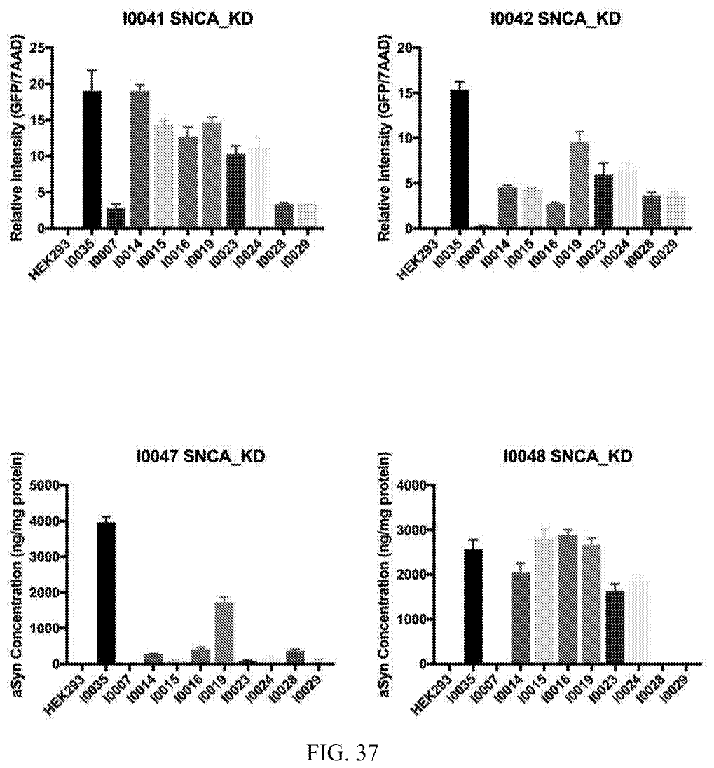

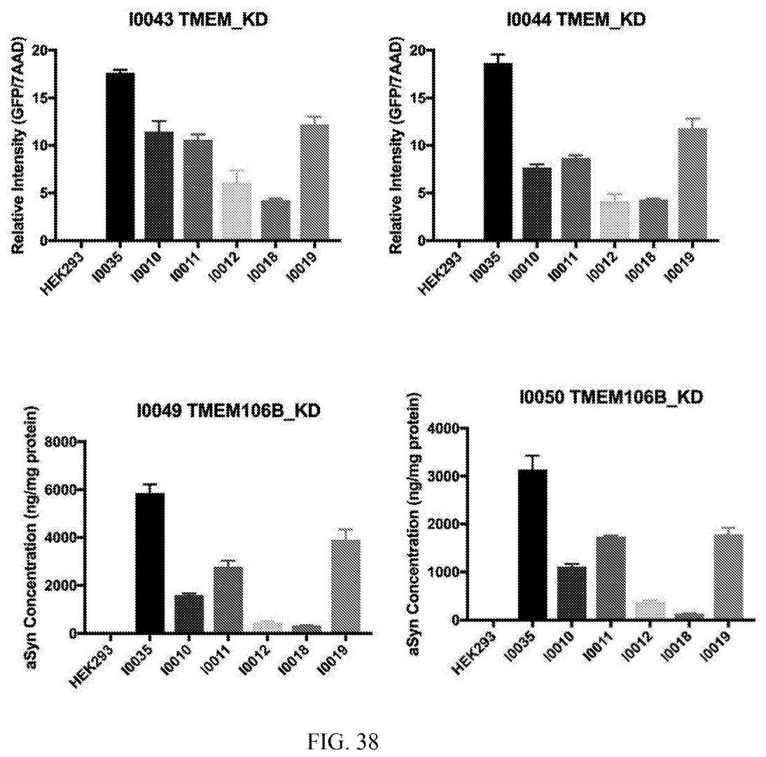

FIG. 37 and Table 9 show representative data indicating successful silencing of SNCA in vitro by GFP reporter assay (top) and α-Syn assay (bottom). FIG. 38 and Table 10 show representative data indicating successful silencing of TMEM106B in vitro by GFP reporter assay (top) and α-Syn assay (bottom).

TABLE 9

| ID | Promoter | Knockdown | Promoter | Overexpress |
|---|---|---|---|---|
| I00007 | CMV_intronic | SNCA_mi | CMV | opt-GBA1 |
| I00008 | H1 | SNCA_sh | CMV | opt-GBA1 |
| I00009 | H1 | SNCA_Pubsh4 | CMV | opt-GBA1 |
| I00014 | JL_intronic | SNCA_mi | JetLong | opt- |
| | | | | SCARB2_GBA |
| I00015 | JL_intronic | SNCA_mi | JetLong | opt- |
| | | | | PSAP_GBA |
| I00016 | JL_intronic | SNCA_mi | JetLong | opt- |
| | | | | CTSB_GBA |
| I00019 | JL_intronic | SNCA_TMEM_mi | JetLong | opt-VPS35 |
| I00023 | JL_intronic | SNCA_mi | JetLong | opt- |
| | | | | GBA1_IL34 |
| I00024 | JL_intronic | SNCA_mi | JetLong | opt-GBA2 |
| I00028 | intronic | SNCA_Broadsh | CMV | opt-GBA1 |
| I00029 | intronic | SNCA_Pubsh4 | CMV | opt-GBA1 |

TABLE 10

| ID | Promoter | Knockdown | Promoter | Overexpress |
|---|---|---|---|---|
| I00010 | H1 | TMEM_Pubsh | CMV | opt-GRN |
| I00011 | JL_intronic | TMEM_mi | JetLong | opt- |
| | | | | GBA1_GRN |
| I00012 | H1 | TMEM_sh | CMV | opt-GRN |
| I00019 | JL_intronic | SNCA_TMEM_mi | JetLong | opt-VPS35 |

### Example 11: ITR “D” Sequence Placement and Cell Transduction

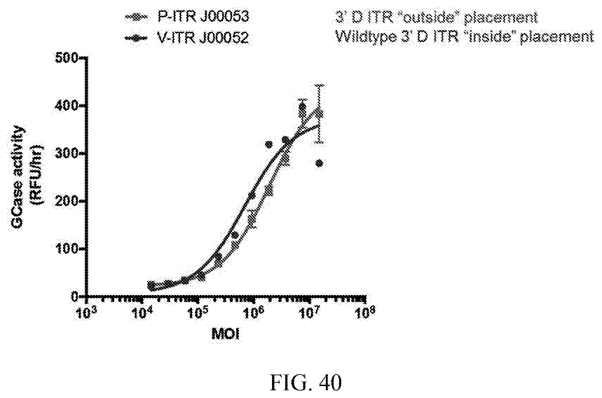

The effect of placement of ITR “D” sequence on cell transduction of rAAV vectors was investigated. HEK293 cells were transduced with Gcase-encoding rAAVs having 1) wild-type ITRs (e.g., “D” sequences proximal to the transgene insert and distal to the terminus of the ITR) or 2) ITRs with the “D” sequence located on the “outside” of the vector (e.g., “D” sequence located proximal to the terminus of the ITR and distal to the transgene insert), as shown in FIG. 20. Data indicate that rAAVs having the “D” sequence located in the “outside” position retain the ability to be packaged and transduce cells efficiently (FIG. 40).

### Example 12: In Vitro Testing of Progranulin rAAVs

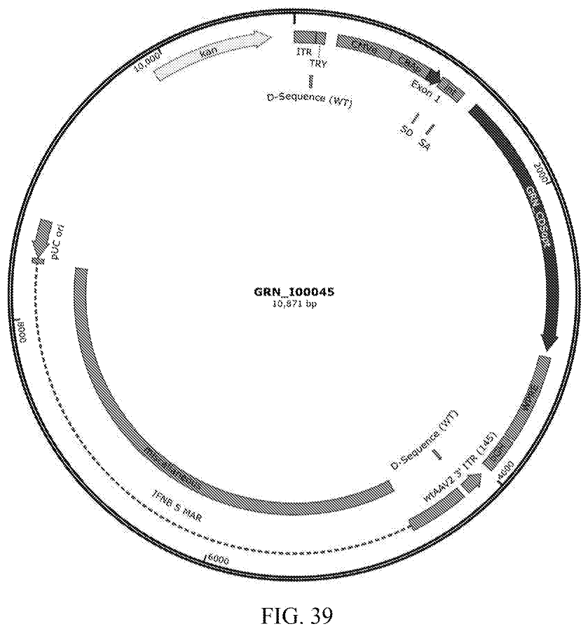

FIG. 39 is a schematic depicting one embodiments of a vector comprising an expression construct encoding PGRN (also referred to as GRN). Progranulin is overexpressed in the CNS of rodents deficient in GRN, either heterozygous or homozygous for GRN deletion, by injection of an rAAV vector encoding PGRN (e.g., codon-optimized PGRN, also referred to as codon-optimized GRN), either by intraparenchymal or intrathecal injection such as into the cisterna magna.

Mice are injected at 2 months or 6 months of age, and aged to 6 months or 12 months and analyzed for one or more of the following: expression level of GRN at the RNA and protein levels, behavioral assays (e.g., improved movement), survival assays (e.g., improved survival), microglia and inflammatory markers, gliosis, neuronal loss, Lipofuscinosis, and/or Lysosomal marker accumulation rescue, such as LAMP1. Assays on GRN-deficient mice are described, for example by Arrant et al. (2017) Brain 140: 1477-1465; Arrant et al. (2018) J. Neuroscience 38(9):2341-2358; and Amado et al. (2018) doi:https://doi.org/10.1101/30869; the entire contents of which are incorporated herein by reference.

### Example 13: In Vitro Testing of MAPT rAAVs

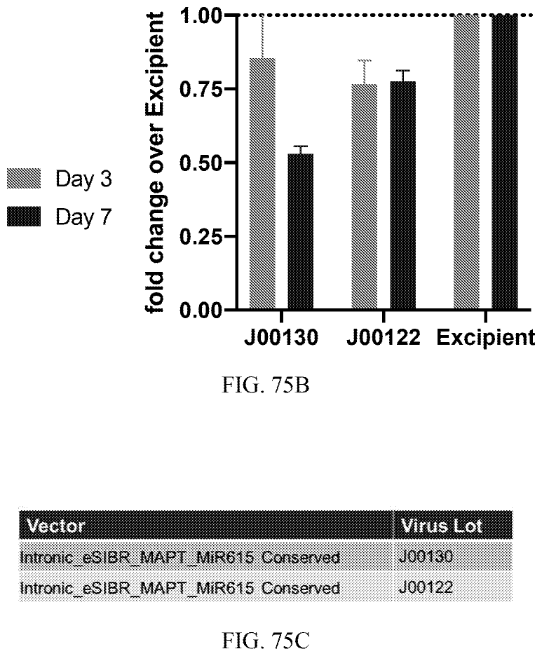

SY5Y cells were plated at 4×10⁴ cells per well in a 96-well plate. The following day, cells were transduced with two virus stocks (Intronic_eSIBR_MAPT_MiR615 Conserved vector) encoding inhibitory RNA targeting MAPT (J00130 produced in a mammalian cell-based system, and J00122 produced in a Baculovirus-based system; shown in FIG. 75C) in triplicates at MOI of 2×10⁵ in media containing 1 uM Hoechst. Excipient alone was used as negative control. The cells were harvested 72 hours later, and stained with a probe to detect AAV vectors expressing inhibitory RNA for MAPT. The probe targets BGHpA. FIG. 75A shows that both virus stocks successfully transduced SY5Y cells.

SY5Y cells were plated at 4×10⁴ cells per well in a 96-well plate. The following day, cells were transduced with two virus stocks ((Intronic_eSIBR_MAPT_MiR615 Conserved vector) encoding inhibitory RNA targeting MAPT (J00130 and J00122; shown in FIG. 75C) in triplicates at MOI 2×10⁶ in media containing 1 uM Hoechst. Excipient alone was used as negative control. SY5Y cells were lysed for RNA extraction 72 hours or 7 days after transduction. cDNA was made from the extracted RNA using Invitrogen Power SYBR Green Cells-to-Ct Kit. qRT-PCR was conducted on cDNA samples and run in triplicates using primers for both human MAPT and GAPDH. FIG. 75B shows data for knockdown of MAPT expression by J00130 and J00122.

### EQUIVALENTS

This application incorporates by reference the contents of the following documents in their entirety: the International PCT Application PCT/US2018/054225, filed Oct. 3, 2018; International PCT Application PCT/US2018/054223, filed Oct. 3, 2018; Provisional Application Ser. No. 62/567,296, filed Oct. 3, 2017, entitled “GENE THERAPIES FOR LYSOSOMAL DISORDERS”; 62/567,311, filed Oct. 3, 2017, entitled “GENE THERAPIES FOR LYSOSOMAL DISORDERS”; 62/567,319, filed Oct. 3, 2017, entitled “GENE THERAPIES FOR LYSOSOMAL DISORDERS”; 62/567,301, filed Oct. 3, 2018, entitled “GENE THERAPIES FOR LYSOSOMAL DISORDERS”; 62/567,310, filed Oct. 3, 2017, entitled “GENE THERAPIES FOR LYSOSOMAL DISORDERS”; 62/567,303, filed Oct. 3, 2017, entitled “GENE THERAPIES FOR LYSOSOMAL DISORDERS”; and 62/567,305, filed Oct. 3, 2017, entitled “GENE THERAPIES FOR LYSOSOMAL DISORDERS”.

Having thus described several aspects of at least one embodiment of this invention, it is to be appreciated that various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

It should also be understood that, unless clearly indicated to the contrary, in any methods claimed herein that include more than one step or act, the order of the steps or acts of the method is not necessarily limited to the order in which the steps or acts of the method are recited.

### SEQUENCES

In some embodiments, an expression cassette encoding one or more gene products (e.g., a first, second and/or third gene product) comprises or consists of (or encodes a peptide having) a sequence set forth in any one of SEQ ID NOs: 1-149. In some embodiments, a gene product is encoded by a portion (e.g., fragment) of any one of SEQ ID NOs: 1-149.

What is claimed is:

1. A method for treating a subject having or suspected of having a central nervous system (CNS) disease, the method comprising administering to the subject an isolated nucleic acid comprising:
(i) an expression construct comprising a transgene encoding one or more gene products listed in Table 1 and/or one or more inhibitory nucleic acids targeting one or more gene products listed in Table 1; and
(ii) two adeno-associated virus (AAV) inverted terminal repeats (ITRs) flanking the expression construct.

2. The method of claim 1, wherein the transgene encodes one or more proteins selected from: GBA1, GBA2, PGRN, TREM2, PSAP, SCARB2, GALC, SMPD1, CTSB, RAB7L, VPS35, GCH1, and IL34.

3. The method of claim 1 or 2, wherein the transgene encoding one or more gene products comprises a codon-optimized protein coding sequence.

4. The method of any one of claims 1 to 3, wherein the transgene encodes one or more inhibitory nucleic acids targeting SNCA, MAPT, RPS25, and/or TMEM106B.

5. The method of any one of claims 1 to 4, wherein the AAV ITRs are AAV2 ITRs.

6. The method of any one of claims 1 to 5, wherein the isolated nucleic acid is packaged into a recombinant adeno-associated virus (rAAV).

7. The method of claim 6, wherein the rAAV comprises an AAV9 capsid protein.

8. The method of any one of claims 1 to 6, wherein the subject is a mammal, optionally wherein the subject is a human.

9. The method of any one of claims 1 to 8, wherein the CNS disease is a neurodegenerative disease, synucleinopathy, tauopathy, and/or lysosomal storage disease (LSD).

10. The method of claim 9, wherein the CNS disease is listed in Table 2, Table 3, Table 4, or Table 5.

11. The method of any one of claims 1 to 10, wherein the administration comprises direct injection to the CNS of the subject, optionally wherein the direct injection is intracerebral injection, intraparenchymal injection, intrathecal injection, intra-cisterna magna injection or any combination thereof.

12. The method of claim 11, wherein the intra-cisterna magna injection is suboccipital injection into the cisterna magna.

13. The method of claim 11 or 12, wherein the direct injection to the CNS of the subject comprises convection enhanced delivery (CED).

14. The method of any one of claims 1 to 13, wherein the administration comprises peripheral injection, optionally wherein the peripheral injection is intravenous injection.

15. The method of any one of claims 6 to 14, wherein the subject is administered about 1×10¹⁰ vg to about 1×10¹⁶ vg of the rAAV.
